(19) United States
(12) Patent Application Publication
(10) Pub. No.: US 2005/0148534 A1
(43) Pub. Date: Jul. 7, 2005

(54) Small molecule compositions and methods for increasing drug efficiency using compositions thereof

(76) Inventors: Angelo John Castellino, San Diego, CA (US); Carlo Ballatore, San Diego, CA (US); Simon Eric Aspland, San Diego, CA (US); Joel Desharnais, San Diego, CA (US); Michael James Newman, San Diego, CA (US); Chengzao Sun, San Diego, CA (US); Peter Wirsching, San Diego, CA (US)

Correspondence Address: FISH & RICHARDSON, PC, 12390 EL CAMINO REAL, SAN DIEGO, CA, 92130-2081, US

(21) Appl. No.: 10/948,364

(22) Filed: Sep. 22, 2004

Related U.S. Application Data
(60) Provisional application No. 60/505,033, filed on Sep. 22, 2003.
(60) Provisional application No. 60/581,835, filed on Jun. 22, 2004.

(51) Int. Cl. A61K 48/00; A61K 31/7076; A61K 31/7072; A61K 31/522; A61K 31/513

(52) U.S. Cl. 514/44; 514/45; 514/49; 514/263.24; 514/269; 514/63; 514/151; 514/81; 514/84; 536/27.1; 536/28.1; 544/243; 544/244; 544/269; 544/309

(57) ABSTRACT

In certain embodiments, provided herein are compositions and methods for increasing drug efficiency. The conjugates provided are in certain embodiments, for compositions and methods in treatment of variety of diseases and have the formula 1:
D-L-S (1) or formula 2:
D-L-S′ (2) wherein D is a drug moiety; L, which may or may not be present, is a non-releasing linker moiety; S is a substrate for a kinase, other than a hexokinase, a protein kinase or a lipid kinase; and S′ is a substrate for a phosphotransferase, other than a hexokinase, a protein kinase or a lipid kinase.

### RELATED APPLICATIONS

Benefit of priority under 35 U.S.C. § 119(e) to U.S. provisional application Ser. No. 60/505,033, filed Sep. 22, 2003, to Aspland et al., entitled “DRUG IMPROVEMENT BY NUCLEOSIDE KINASE SPECIFIC TARGETING AND TRAPPING” and U.S. provisional application Ser. No. 60/581,835, filed Jun. 22, 2004, to Aspland et al., entitled “SMALL MOLECULE COMPOSITIONS AND METHODS FOR INCREASING DRUG EFFICIENCY USING COMPOSITIONS THEREOF” is claimed. The subject matter of the above-referenced applications are incorporated by reference in their entirety.

### FIELD

Conjugates, compositions and methods for improving drug efficiency are provided. The conjugates provided are for delivery of therapeutic agents for treating a variety of disorders, such as, proliferative diseases, autoimmune diseases, infectious diseases and inflammatory diseases, are provided. The conjugates contain drug moieties and substrates for kinases other than hexokinases, protein kinases and lipid kinases non-releasably linked thereto, optionally by a non-releasing linker.

### BACKGROUND

Many potent, but relatively non-specific drugs have been developed for the treatment of cancer. Examples of drugs for the treatment of cancer include taxanes or taxoids, vinca alkaloids, alkylating agents, camptothecins, and anthracyclines. Furthermore, much of modern drug discovery and development is focused on the identification of small molecules which enter cells and inhibit proteins responsible for the genesis or maintenance of the condition being treated, either by down-regulating the expression of the protein or directly inhibiting its function. Another approach inhibits DNA synthesis in proliferating cells by using an anti-metabolite which includes nucleoside analogs and pyrimidine and purine bases.

One method for antimetabolite treatment of cancer uses a pyrimidine containing compound whose action depends upon inhibition of de novo pyrimidine nucleotide biosynthesis. Resistance is common due to upregulation of thymidine synthetase which overcomes inhibition. Another method for antimetabolite treatment of cancer uses a purine containing compound whose action depends upon HGRT, and the most common cause of resistance is the deficiency of this enzyme. Targeting is lacking with antimetabolites used for treatment of cancer as evidenced by the poor discrimination between normal cells and cancer cells. Furthermore, the anti-metabolites used in cancer chemotherapy are prodrugs, since additional metabolic events are needed once cellular entry is gained for a therapeutic effect to be realized. For example, the anti-metabolite 5-FdU, a pyrimidine base, must be converted to a nucleotide for inhibition of thymidine synthetase, which is responsible for the therapeutic effect.

Viral anti-metabolites target the viral thymidine kinase (TK), but trapping or accumulation is not responsible for their therapeutic effect. Viral anti-metabolites acted upon by viral TK include pyrimidine and purine containing compounds. Anti-metabolites used in anti-viral therapy are also prodrugs, and must be subject to downstream intracellular enzymes for conversion to the nucleoside triphosphate, and incorporation into the developing DNA strand to inhibit the DNA synthesizing machinery of a viral infected cell, which is the actual event responsible for the therapeutic effect. Viral diseases treated by anti-metabolites include the DNA viruses HSV-1, HSV-1, VZV, EBV, CMV and the RNA viruses HTLV-1 and HIV. Although HSV infections are well treated with acyclovir, HSV encephalitis is either fatal or results in serious neurological outcomes. RNA viral infections are especially problematic and despite the advances in treating AIDS caused by HIV infection, the disease caused by this virus is invariably fatal.

Thus, the effectiveness of drugs used to treat cancer and viral infections is frequently limited by side effects produced in cells not directly involved in the genesis or maintenance of the disease being treated. Drug effectiveness can also be limited by resistance to the drug which develops during treatment. This resistance is exemplified by the treatment of cancer wherein drug is actively removed from the treated cell by a P-glycoprotein transporter or wherein the effectiveness of the drug is diminished by over-expression of the enzyme upon which the drug acts.

Accordingly, considerable efforts have been directed to effect targeting of anti-cancer to tumors since many anti-cancer compounds of chemical origin are extremely potent and able to kill virtually any cell. Yet current protocols generally require high concentrations and/or prolonged administration of agents, or only result in low and/or variable concentrations in the cytosol or nucleus of the cell and therefore give inefficient cytotoxicity and/or significant systemic toxicity. Similarly, a significant need to improve drug efficiency of anti-viral drugs exists.

### SUMMARY

Provided herein are compounds and methods for targeted delivery of drugs. The compounds are conjugates that contain a drug moiety and a substrate for a kinase other than a hexokinase, a protein kinase or a lipid kinase non-releasably linked thereto. The drug moieties include therapeutic agents, such as a cytotoxic agents, and diagnostic agents, such as labeled moieties and imaging agents other than compounds containing a carboranyl, hydroxyboryl and rare earth crypate moiety. The substrates are substrates for a kinase other than a hexokinase, a protein kinase or a lipid kinase. In certain embodiments, the drug moiety is a therapeutic agent other than a compound containing a carboranyl or hydroxyboryl moiety. In certain embodiments, the drug moiety is a label other than a compound containing a rare earth crypate moiety.

The conjugates contain one or more substrates for one or a plurality of kinases other than a hexokinase, a protein kinase or a lipid kinase non-releasably linked thereto, either directly or via a non-releasing linker to a drug moiety, such as a cytotoxic agent. The conjugates provided herein contain the following components: (substrate)ₜ, (linker)_{q}, and (drug)_{d} in which: at least one substrate for a kinase other than a hexokinase, a protein kinase or a lipid kinase is non-releasably linked, optinally via a linker, to a drug moiety. t is 1 to 6 and each substrate is the same or different, and is generally 1 or 2; q is 0 to 6; 0 to 4; 0 or 1; d is 1 to 6, in certain embodiment 1 or 2 and each drug moieties are the same or different; linker refers to any non-releasing linker; and the drug is any therapeutic agent, such as a cytotoxic agent, including an anti-cancer drug, a diagnostic agent, such as an imaging agent or labeled moiety other than compounds containing a carboranyl, hydroxyboryl and rare earth crypate moiety. The drug moiety of the drug conjugate may be derived from a naturally occurring or synthetic compound that may be obtained from a wide variety of sources, including libraries of synthetic or natural compounds. Exemplary drug moieties can be cytotoxic agents, including, but not limited to, anti-infective agents, antihelminthic, antiprotozoal agents, antimalarial agents, antiamebic agents, antileiscmanial drugs, antitrichomonal agents, antitrypanosomal agents, sulfonamides, antimycobacterial drugs, or antiviral chemotherapeutics.

The conjugates for use in the compositions and methods provided herein have formula (1):
(D)_{d}-(L)_{q}-(S)ₜ (1)
or a pharmaceutically acceptable derivative thereof, wherein D is a drug moiety; d is 1-6, or is 1 or 2; L is a non-releasing linker; q is 0 to 6, or is 0 or 1; S is a substrate for a kinase other than a hexokinase, a protein kinase or a lipid kinase; and t is 1 to 6, or is 1 or 2, or is 1. In the conjugates, the drug moiety is covalently attached, optionally via a non-releasing linker, to the substrate. In the conjugates provided herein, the conjugation of the drug moiety(s) or non-releasing linker linked thereto can be at various positions of the substrate.

In the conjugates that contain two drug moieties, which are the same or different, conjugation to the drug moiety(s) or non-releasing linker linked thereto can be at various positions of the substrate.

In certain embodiments, the kinase is overexpressed, overactive or exhibits undesired activity in a target system. The action of the kinase on the substrate results in a negative charge on the conjugate. The action of the kinase on the substrate may result in improved drug efficiency.

The target system may be a cell, tissue or organ. In particular embodiments, the cell is a tumor cell or a tumor-associated endothelial cell. The target system may also be associated with cancer, inflammation, angiogenesis, autoimmune syndromes, transplant rejection or osteoporosis.

The substrate, in certain embodiments, has a molecular weight of between about 50 amu and 1000 amu. In other embodiments, the substrate has a molecular weight of more than 1000 amu such as when the substrate exists as a dimer.

In certain embodiments, the conjugates have formula (2)
D-L-S′ (2)
wherein D and L are as defined in formula (1); and

S′ is a substrate for a phosphotransferase other than a hexokinase, a protein kinase or a lipid kinase. In certain embodiments, contemplated phosphotransferase are designated by the Enzyme Commission under the general category number EC 2.7.1 with the exceptions of the specific EC numbers 2.7.1.1 (hexokinase), 2.71.37 (protein kinase), 2.7.1.91 (sphinganine kinae) and EC numbers designating other lipid kinases. In one embodiment the phospho group acceptor is a nucleoside. The substrate in the conjugates herein can be a substrate for a kinase including, but not limited to, thymidine kinase, viral thymidine kinase, TK-1, deoxycytidine kinase, deoxyguanosine kinase.

The substrate, in certain embodiments, is phosphorylated upon action of a kinase such as thymidine kinase, viral thymidine kinase, TK-1, deoxycytidine kinase, deoxyguanosine kinase. In certain embodiments, the substrate is nucleoside. Examples of nucleosides for use as substrates in the conjugates provided herein include, but are not limited to, cytosine, uridine, thymidine, guanosine, adenosine, or derivatives thereof.

In the above formula 1, the drug moiety can be a hydrophobic drug. In certain embodiments, D can be a detectable label. In certain embodiments, the drug is an anti-cancer drug.

Pharmaceutical compositions containing a conjugate of formula 1 and a pharmaceutically acceptable carrier are provided herein.

Also provided are methods for using the conjugates. The methods provided are methods for treating conditions caused by undesirable chronic or aberrant cellular activation, migration, proliferation or survival (ACAMPS). Furthermore, methods for ameliorating a cell-proliferative disorder including, but not limited to, cancer are also provided. In one embodiment, the conjugates are for used in methods for treating cancer.

Also provided are methods of improving drug efficiency by administering a therapeutically effective amount of a conjugate provided herein to a target system or organism, wherein the action of the kinase on the substrate results in improved drug efficiency.

In one embodiment, methods for identifying kinase substrates capable of selectively accumulating in a target system are provided. The methods contain the steps of: a) contacting one or more conjugates with a kinase that is overexpressed, overactive or that exhibits undesired activity in a target system; and b) determining kinase activity on one or more conjugates. In other embodiment, the method for identifying kinase substrates capable of selectively accumulating in a target system further contains the steps of: c) determining a first amount or a plurality of first amounts of one or more conjugates in the target system; and d) determining a second amount or a plurality of second amounts of one or more conjugates in a non-target system.

In one example, one or more conjugates may contain a detectable label. For example, the label may be radioactive or fluorescent. The radioactive lable is a radioactive compound other than a compound containing rare earth crypate moiety.

The target system may be associated with cancer, inflammation, angiogenesis, autoimmune syndromes, transplant rejection or osteoporosis. The target system may be a cell, tissue or organ. In one embodiment, the cell may be a tumor cell or a tumor-associated endothelial cell.

In one embodiment, methods for identifying conjugates capable of exhibiting selective toxicity against a target system are provided. The methods contain the steps of: a) contacting one or more conjugates containing a drug moiety with a target system; and b) determining the cytotoxicity of the one or more conjugates against the target system.

### DETAILED DESCRIPTION OF EMBODIMENTS

A. Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The singular forms “a,” “an,” and “the” include plural references, unless the context clearly dictates otherwise. Thus, for example, references to a composition for delivering “a drug” include reference to one, two or more drugs.

As used herein, “drug conjugate” or a “conjugate” refers to compounds having one or more drug moieties non-releasably linked, optionally via a non-releasable linker, to a substrate for one or more kinase other hexokinase, a protein kinase or a lipid kinase. The drug-substrate conjugates provided herein retain a significant fraction of drug activity within the conjugate and the desired therapeutic effect is elicited by the drug-substrate conjugate without having the need to cleave the drug from the substrate. In certain embodiments, the drug moiety or the substrate moiety in the conjugate can be present in a form of a pharmaceutically acceptable derivative that renders the conjugate biologically inactive. The inactive drug-substrate conjugate can be converted to the active drug-substrate conjugate under physiological conditions without having the need to cleave the drug-substrate conjugate.

As used herein, “substrate” is a molecule which is subject to phosphorylation by an enzyme, other than a hexokinase, a protein kinase or a lipid kinase, and encompasses species which can be converted by chemical and/or enzymatic reaction(s) to a substrate upon or after introduction of the molecule (in conjugate form) to a target system or organism. The substrates for use herein include, but are not limited to substrates for nucleoside kinases such as thymidine kinase, deoxycytidine kinase and deoxyguanosine kinase. The substrates for nuceoside kinases include, but are not limited to, natural and non-natural nucleosides and their analogs, and natural and non-natural bases for nucleosides, such as purines and pyrimidines and their analogs.

As used herein, “drug” or “drug moiety” is any drug or other agent that is intended for delivery to a targeted cell or tissue, such as cells or tissues associated with aberrant cellular activation, migration, proliferation or survival, other than a compound containing a carboranyl, hydroxyboryl or rare earth cryptate containing moiety. Drug moiety for use herein, include, but are not limited to, anti-cancer agents, anti-angiogenic agents, cytotoxic agents and labels other than compounds containing a carboranyl, hydroxyboryl or rare earth cryptate containing moieties, as described herein and known to those of skill in the art.

As used herein, an anti-cancer agent (used interchangeably with “anti-tumor or anti-neoplasm agent”) refers to any agents used in the treatment of cancer. These include any agents, when used alone or in combination with other compounds, that can alleviate, reduce, ameliorate, prevent, or place or maintain in a state of remission of clinical symptoms or diagnostic markers associated with neoplasm, tumor or cancer, and can be used in methods, combinations and compositions provided herein. Non-limiting examples of anti-neoplasm agents include anti-angiogenic agents, alkylating agents, antimetabolite, certain natural products that are anti-neoplasm agents, platinum coordination complexes, anthracenediones, substituted ureas, methylhydrazine derivatives, adrenocortical suppressants, certain hormones, antagonists and anti-cancer polysaccharides.

As used herein, anti-angiogenic agent refers to any compound, that, when used alone or in combination with other treatment or compounds, can alleviate, reduce, ameliorate, prevent, or place or maintain in a state of remission, one or more clinical symptoms or diagnostic markers associated with undesired and/or uncontrolled angiogenesis. Thus, for purposes herein an anti-angiogenic agent refers to an agent that inhibits the establishment or maintenance of vasculature. Such agents include, but are not limited to, anti-tumor agents, and agents for treatments of other disorders associated with undesirable angiogenesis, such as diabetic retinopathies, hyperproliferative disorders and others.

As used herein, “drug-linker construct” refers to a chemical combination wherein a drug moiety and a linker moiety are covalently attached. Similarly, a “drug-substrate construct” refers to a chemical combination wherein a drug moiety and a substrate moiety are covalently attached.

As used herein, “linker-substrate construct” refers to a chemical combination wherein a linker moiety and a substrate moiety are covalently attached.

As used herein, the term “fraction of activity” refers to an amount of the desired biological activity of a test compound, such as a drug-substrate conjugate provided herein, compared with the biological activity of the unconjugated drug or unconjugated substrate. The desired biological activity for the conjugates, the parent drugs or the substrates can be measured by any method known in the art, including, but not limited to, cytotoxicity assay, tubulin polymerisation assay and thymidine kinase activity assays described herein. In certain embodiments, the biological activity of the conjugates provided herein is greater than the activity of the parent drug moiety. As used herein a “significant fraction” referes to the activity of from about 5% up to about 100% of the biological activity, from about 5% up to about 95%, from about 5% up to about 90%, from about 5% up to about 80%, up to about 70%, up to about 60%, or up to about 50% of the biological activity. Significant fraction is also mean to include biological activity of 100% or more.

As used herein “subject” is an animal, typically a mammal, including human, such as a patient.

As used herein, “aberrant” refers to any biological process, cellular activation, migration, proliferation or survival, enzyme level or activity that is in excess of that associated with normal physiology.

As used herein, “chronic” refers to a biological process, cellular activation, migration, proliferation or survival, enzyme level or activity that is persistent or lasts longer than that associated with normal physiology.

As used herein, “undesirable” refers to normal physiological processes that occur at an undesirable time, such as but not limited to, immune responses associated with transplant rejection and/or graft versus host disease.

As used herein, “ACAMPS” refers to aberrant cellular activation, migration, proliferation or survival. ACAMPS conditions are characterized by undesirable or aberrant activation, migration, proliferation or survival of tumor cells, endothelial cells, B cells, T cells, macrophages, granulocytes including neutrophils, eosinophils and basophils, monocytes, platelets, fibroblasts, other connective tissue cells, osteoblasts, osteoclasts and progenitors of many of these cell types. Examples of ACAMPS-related conditions include, but are not limited to, cancer, coronary restenosis, osteoporosis and syndromes characterized by chronic inflammation and/or autoimmunity.

As used herein, “hydrophobic drug” refers to any organic or inorganic compound or substance having biological or pharmaceutical activity with water solubility of less than 100 mg/ml, having a log P greater than 2, being lipid soluble or not adsorbing water.

As used herein, the term “effective amount of therapeutic response” refers to an amount which is effective in prolonging the survivability of the patient beyond the survivability in the absence of such treatment. Prolonging survivability also refers to improving the clinical disposition or physical well-being of the patient. When used in reference to cancer treatment methods, the term “therapeutically effective amount” refers to an amount which is effective, upon single or multiple dose administration to the patient, in controlling tumor growth. As used herein, “controlling tumor growth” refers to slowing, interrupting, arresting or stopping the migration or proliferation of tumor or tumor-associated endothelial cells.

The cytotoxic selectivity of the conjugates provided herein is assessed by comparing conjugate cytotoxicity against normal cells to the conjugate cytotoxicity in the tumor cells. Typically, the conjugates show highter cytotoxicity selectivity for tumor cells as compared to the normal cells. As used herein, the term “cytotoxic selectivity index” refers to the ratio of EC₅₀ of the conjugate in tumor cells to the EC₅₀ of the conjugate in normal cell. In certain embodiments, the conjugates provided herein have higher cytotoxic selectivity for tumor cells than that of the parent drug. In certain embodiments, the conjugates provided herein show inproved cytotoxic selectivity index as compared to the parent drug. The cytotoxic selectivity index values for the conjugates provided herein are calculated by the methods provided herein.

As used herein, the term “improved drug efficiency” refers to a property of a drug within the conjugate which is improved relative to the drug in free form. Improved drug efficiency includes, but is not limited to, increased solubility, altered pharmacokinetics, including adsorption, distribution, metabolism and excretion, an increase in maximum tolerated dose, a reduction of side effects, an increase in cytotoxic selectivity index, an ability to surmount or avoid resistance mechanisms, or an ability to be administered chronically or more frequently. For example, a more efficient drug may have an improved cytotoxic selectivity index as compared to a less efficient drug. In certain embodiments, the improvement in the cytotoxic selectivity index is at least 1.5 fold greater is the conjugate.

As used herein, “non releasing linker moiety” or “non releasable linker moiety” refers to a linker moiety that is attached to a drug moiety through a covalent bond or functionality which remains substantially intact under physiological conditions during a period of time required for eliciting a pharmacological response such that the pharmacological response is not due to free drug. Typically, the time is sufficient for uptake of the conjugate by the target system. In certain embodiments, the linkage remains from about 10% up to about 100% intact under physiologic conditions in a period of about 0.1 hours up to about 3 hours. In certain embodiments, the linker is more than 50% intact, in another embodiment, more than 60%, more than 70%, 80% or 90% intact. Evaluation of the stability of such linkage can be made by one of skill in the art using methods known in the art.

As used herein, “linker moiety” refers to the intervening atoms between the drug moiety and substrate. A linker precursor, used interchangeably with linker precursor moity, is a compound that is used in the synthesis of a drug linker construct or a substrate linker construct. The terms “linker” and “linking moiety” herein refer to any moiety that non-releasably connects the substrate moiety and drug moiety of the conjugate to one another. The linking moiety can be a covalent bond or a chemical functional group that directly connects the drug moiety to the substrate. The linking moiety can contain a series of covalently bonded atoms and their substituents which are collectively referred to as a linking group. Linking moieties are characterized by a first covalent bond or a chemical functional group that connects the drug moiety to a first end of the linker group and a second covalent bond or chemical functional group that connects the second end of the linker group to the substrate. The first and second functionality, which independently may or may not be present, and the linker group are collectively referred to as the linker moiety. The linker moiety is defined by the linking group, the first functionality if present and the second functionality if present. As used herein, the linker moiety contains atoms interposed between the drug moiety and substrate, independent of the source of these atoms and the reaction sequence used to synthesize the conjugate.

As used herein “non-releasably linked” refers to linkage of a drug moiety through a covalent bond or functionality wherein the linkage remains substantially intact under physiological conditions during a period of time required for eliciting a pharmacological response such that the pharmacological response is not due to free drug. In certain embodiments, the linkage remains from about 10% up to about 100% intact under physiologic conditions in a period of about 0.1 hours up to about 3 hours. In certain embodiments, the linker is more than 50% intact, in another embodiment, more than 60%, more than 70%, 80% or 90% intact.

In the conjugates provided herein, in certain embodiments, L′, L″, etc. refers to linker groups or covalent bonds that connect the first and the second functionalities of the linker or the linking moiety.

As used herein, “label” or “labeling agent”is a molecule that allows for the manipulation and/or detection of the conjugate which contains the label. Examples of labels include spectroscopic probes such as chromophores, fluorophores, and contrast agents. Other spectroscopic probes have magnetic or paramagnetic properties. The label may also be a radioactive molecule or a molecule that is part of a specific binding pair well known in the art such as biotin and streptavidin. The radioactive lable for use herein is a radioactive compound other than a compound containing a rare earth crypate moiety.

The term “nucleoside” as used herein, refers to a molecule composed of a heterocyclic base and a carbohydrate. Typically, a nucleoside is composed of a heterocyclic nitrogenous base in N-glycosidic linkage with a sugar. Nucleosides are recognized in the art to include natural bases (standard), and non-natural bases well known in the art. The carbohydrates include the true sugars found in natural nucleosides or a species replacing the ribofuranosyl moiety or acyclic sugars. The heterocyclic nitrogenous bases are generally located at the 1′ position of a nucleoside sugar moiety. Nucleosides generally contain a base and sugar group. The nucleosides can be unmodified or modified at the sugar, and/or base moiety, (also referred to interchangeably as nucleoside analogs, modified nucleosides, non-natural nucleosides, non-standard nucleosides; see for example, Eckstein et al., International PCT Publication No. WO 92/07065 and Usman et al., International PCT Publication No. WO 93/15187). In natural nucleosides the heterocyclic base is typically thymine, uracil, cytosine, adenine or guanine. The carbohydrate shall be understood to mean the true sugar found in natural nucleosides or a species replacing the ribofuranosyl moiety or acyclic sugars. In certain embodiments, acyclic sugars contain 3-6 carbon atoms and include, for example, the acyclic sugar moieties present in acyclovir (—CH2-O—CH2CH2-OH), ganciclovir (—CH2-O—CH(CH2OH)—CH2-OH), and the like. Natural nucleosides have the β-D-configuration. The term “nucleoside” shall be understood to encompass unnatural configurations and species replacing the true sugar that lack an anomeric carbon. In natural nucleosides the heteocyclic base is attached to the carbohydrate through a carbon-nitrogen bond. The term “nucleoside” shall be understood to encompass species wherein the heterocyclic base and carbohydrate are attached through a carbon-carbon bond (C-nucleosides).

As used herein, “target system” is a cell, tissue or organ which is responsible for the genesis or maintenance of a disease state or is responsible for or associated with the condition being treated.

As used herein, biological activity refers to the in vivo activities of a compound or physiological responses that result upon in vivo administration of a compound, composition or other mixture. Biological activity, thus, encompasses therapeutic effects and pharmacokinetic behaviour of such compounds, compositions and mixtures. Biological activities can be observed in in vitro systems designed to test for such activities.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl ar heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl ar heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating a cancer.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, EC₅₀ refers to a dosage, concentration or amount of a particular test compound that elicits a dose-dependent response at 50% of maximal expression of a particular response that is induced, provoked or potentiated by the particular test compound.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound. The instant disclosure is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, the nomenclature alkyl, alkoxy, carbonyl, etc. is used as is generally understood by those of skill in this art.

As used herein, alkyl, alkenyl and alkynyl carbon chains, if not specified, contain from 1 to 20 carbons, or 1 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, ethene, propene, butene, pentene, acetylene and hexyne. As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “cycloalkyl” refers to a saturated mono- or multicyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “substituted alkyl,” “substituted alkenyl,” “substituted alkynyl,” “substituted cycloalkyl,” “substituted cycloalkenyl,” and “substitued cycloalkynyl” refer to alkyl, alkenyl, alkynyl, cycloalkyl, cycloalkenyl and cycloalkynyl groups, respectively, that are substituted with one or more substituents, in certain embodiments one to three or four substituents, where the substituents are as defined herein, generally selected from Q¹.

As used herein, “aryl” refers to aromatic monocyclic or multicyclic groups containing from 6 to 19 carbon atoms. Aryl groups include, but are not limited to groups such as fluorenyl, substituted fluorenyl, phenyl, substituted phenyl, naphthyl and substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrrolidinyl, pyrimidinyl, tetrazolyl, thienyl, pyridyl, pyrrolyl, N-methylpyrrolyl, quinolinyl and isoquinolinyl.

As used herein, a “heteroarylium” group is a heteroaryl group that is positively charged on one or more of the heteroatoms.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

As used herein, “substituted aryl,” “substituted heteroaryl” and “substituted heterocyclyl” refer to aryl, heteroaryl and heterocyclyl groups, respectively, that are substituted with one or more substituents, in certain embodiments one to three or four substituents, where the substituents are as defined herein, generally selected from Q¹.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyano, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1-chloro-2-fluoroethyl.

As used herein, “haloalkoxy” refers to RO— in which R is a haloalkyl group.

As used herein, “sulfinyl” or “thionyl” refers to —S(O)—. As used herein, “sulfonyl” or “sulfuryl” refers to —S(O)₂—. As used herein, “sulfo” refers to —S(O)₂O—.

As used herein, “carboxy” refers to a divalent radical, —C(O)O—.

As used herein, “aminocarbonyl” refers to —C(O)NH₂.

As used herein, “alkylaminocarbonyl” refers to —C(O)NHR in which R is alkyl, including lower alkyl. As used herein, “dialkylaminocarbonyl” refers to —C(O)NR′R in which R′ and R are independently alkyl, including lower alkyl; “carboxamide” refers to groups of formula —NR′COR in which R′ and R are independently alkyl, including lower alkyl.

As used herein, “diarylaminocarbonyl” refers to —C(O)NRR′ in which R and R′ are independently selected from aryl, including lower aryl, such as phenyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

As used herein, “alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl or COR′, where R′ is alkyl, aryl, aralkyl, heteroaryl, heteroaralkyl, —OY or —NYY′, where Y and Y′ are each independently hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “azaalkylene” refers to —(CRR)ₙ—NR—(CRR)ₘ—, where n and m are each independently an integer from 0 to 4. As used herein, “oxaalkylene” refers to —(CRR)ₙ—O—(CRR)ₘ—, where n and m are each independently an integer from 0 to 4. As used herein, “thiaalkylene” refers to —(CRR)ₙ—S—(CRR)ₘ, —(CRR)ₙ—S(═O)—(CRR)ₘ—, and —(CRR)ₙ—S(═O)₂—(CRR)ₘ—, where n and m are each independently an integer from 0 to 4. In certain embodiments herein, the “R” groups in the definitions of azaalkylene, oxaalkylene and thiaalkylene are each independently selected from hydrogen and Q¹, as defined herein.

As used herein, “alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C≡C—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in one embodiment having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur or substituted or unsubstituted nitrogen atoms, where the nitrogen substituent is alkyl. Alk(en)(yn)ylene groups include, but are not limited to, —C═C—(CH₂)ₙ—C≡C—, where n is 1 or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “cycloalkylene” refers to a divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fused, bridged or spiro-connected fashion. “Cycloalk(en)(yn)ylene” refers to a cycloalkylene group containing at least one double bond and at least one triple bond.

As used herein, “substituted alkylene,” “substituted alkenylene,” “substituted alkynylene,” “substituted cycloalkylene,” “substituted cycloalkenylene,” and “substitued cycloalkynylene” refer to alkylene, alkenylene, alkynylene, cycloalkylene, cycloalkenylene and cycloalkynylene groups, respectively, that are substituted with one or more substituents, in certain embodiments one to three or four substituents, where the substituents are as defined herein, generally selected from Q¹.

As used herein, “arylene” refers to a monocyclic or polycyclic, in certain embodiments monocyclic, divalent aromatic group, in one embodiment having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 5 or 6 carbons.

As used herein, “heteroarylene” refers to a divalent monocyclic or multicyclic aromatic ring system, in one embodiment of about 5 to about 15 members where one or more, in certain embodiments 1 to 3, of the atoms in the ring system is a heteroatom, that is, an element other than carbon, including but not limited to, nitrogen, oxygen or sulfur.

As used herein, “heterocyclylene” refers to a divalent monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including but not limited to, nitrogen, oxygen or sulfur.

As used herein, “substituted arylene,” “substituted heteroarylene” and “substituted heterocyclylene” refer to arylene, heteroarylene and heterocyclylene groups, respectively, that are substituted with one or more substituents, in certain embodiments one to three of four substituents, where the substituents are as defined herein, generally selected from Q¹.

As used herein, “alkylidene” refers to a divalent group, such as ═CR′R″, which is attached to one atom of another group, forming a double bond. Alkylidene groups include, but are not limited to, methylidene (═CH₂) and ethylidene (═CHCH₃). As used herein, “arylalkylidene” refers to an alkylidene group in which either R′ or R″ is an aryl group. “Cycloalkylidene” groups are those where R′ and R″ are linked to form a carbocyclic ring. “Heterocyclylidene” groups are those where at least one of R′ and R″ contain a heteroatom in the chain, and R′ and R″ are linked to form a heterocyclic ring.

As used herein, “amido” refers to the divalent group —C(O)NH—. “Thioamido” refers to the divalent group —C(S)NH—. “Oxyamido” refers to the divalent group —OC(O)NH—. “Thiaamido” refers to the divalent group —SC(O)NH—. “Dithiaamido” refers to the divalent group —SC(S)NH—. “Ureido” refers to the divalent group —HNC(O)NH—. “Thioureido” refers to the divalent group —HNC(S)NH—. As used herein, aminocarbonyl refers to —NHC(O) group. As used herein, aminocarbonyloxy refers to —NHC(O)O— group.

As used herein, “semicarbazide” refers to —NHC(O)NHNH, “thiosemicarbizide refers to —NHC(S)NHNH. “Carbazate” refers to the divalent group —OC(O)NHNH—. “Isothiocarbazate” refers to the divalent group —SC(O)NHNH—. “Thiocarbazate” refers to the divalent group —OC(S)NHNH—. “Sulfonylhydrazide” refers to the group —SO₂NHNH—. “Hydrazide” refers to the divalent group —C(O)NHNH—. “Azo” refers to the divalent group —N═N—. “Hydrazinyl” refers to the divalent group —NH—NH—.

Where the number of any given substituent is not specified (e.g., “haloalkyl”), there may be one or more substituents present. For example, “haloalkyl” may include one or more of the same or different halogens. As another example, “C₁₋₃alkoxyphenyl” may include one or more of the same or different alkoxy groups containing one, two or three carbons.

As used herein, PEG linker represents a polyethylene glycol chain containing the designated number of atoms in the chain between the drug moiety and the substrate, conjugated to the drug moiety at the first end and to the substrate at the second end.

As used herein, alkane linker represents an alkylene group having the designated number of atoms in the chain between the drug moiety and the substrate, conjugated to the drug moiety at the first end and to the substrate at the second end.

The following naming conventions have been used to name the conjugates provided herein:

The conjugates are named in four parts: “Drug”-“Point of Attachment and functionality to the “Drug”-“Linker Type (Linker Length)”-“Enzyme Substrate”. In an exemplary conjugate, thymidine as the enzyme substrate is attached to the linker at N3 of the nucleoside base.

The drug moieties in exemplary conjugates provided herein have been abbreviated as follows:
Paclitaxel or O¹⁰ deacetyl-paclitaxel=PXL
Vinblastine or O⁴-deacetyl-=VBL
Doxorubicin=DOX

In naming the conjugates, the abbreviated name of the drug is followed by the point of attachment and functionality linking the drug to the substrate, optinally via linking atoms interspaced inbetween. The point of attachment to the substrate is indicated as a prefix to the substrate abrreviation. For example, conjugate PXL-7Ca-ALK(6)-N-3-THY is a paclitaxel thymidine conjugate, wherein N3 of thymidine is conjugated to paclitaxel at C7 with a C6 alkane unit via a carbamate functionality. Table 1 provides examples of various drug moieties with possible points of attachments and linking functionalities. Table 2 herein provides examples of various linker groups and the names thereof.

As used herein, the following terms have their accepted meaning in the chemical literature:
AcOH acetic acid
CHCl₃ chloroform
conc concentrated
DBU 1,8-diazabicyclo[5.4.0]undec-7-ene
DIEA N-ethyl-N,N-di-isopropylamine
DCM dichloromethane
DME 1,2-dimethoxyethane
DMF N,N-dimethylformamide
DMSO dimethylsulfoxide
EtOAc ethyl acetate
EtOH ethanol (100%)
Et₂O diethyl ether
Hex hexanes
H₂SO₄ sulfuric acid
MeCN acetonitrile
MeOH methanol
Pd/C palladium on activated carbon
TEA triethylamine
THF tetrahydrofuran
TFA trifluoroacetic acid

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11: 942-944).

B. Conjugates

Provided herein are drug-substrate conjugates for use in the methods and compositions for increasing drug efficiency. The drug-substrate conjugates provided herein retain a significant fraction of parent drug activity within the conjugate and the desired therapeutic effect is elicited by the drug-substrate conjugate without having the need to cleave the drug from the substrate.

The conjugates provided herein are not limited to specific drug, linker and substrate moieties. Various combinations of the drug, linker and substrate moieties can be prepared using synthetic methodologies known in the art and described herein. As discussed above, the conjugates can contain a plurality of substrates, a plurality of linkers and a plurality of drug moieties.

In certain embodiments, the conjugates provided herein retain a significant fraction of biological activity of parent drug within the conjugate. In certain embodiments, the conjugates retain from about 5% up to about 100% of the biological activity, from 5% up to about 95%, from about 5% up to about 90%, from about 5% up to about 80%, up to about 70%, up to about 60% or up to about 50% of the biological activity of parent drug. In certain embodiment the biological activity of the drug in the conjugate exceeds that of parent drug.

Without being bound to any theory, in certain embodiments, the drug-substrate conjugates are selectively trapped or accumulated in target cells. In certain embodiments, the conjugates are selectively trapped or accumulated in target cells due to phosphorylation of the substrate in the conjugates by a kinase whose activity is involved in the condition being treated. As a result, doses of the drug-substrate conjugate required to elicit the same effective amount of therapeutic response as the parent drug can be reduced thereby resulting in a reduction of undesirable side effects. This allows for an increase in the duration of therapy, which is highly desirable in chronic disease settings. In addition, the standard drug dose in conjugate form can be increased without exceeding the tolerability of undesirable side effects to allow for more aggressive treatment. Furthermore, molecules capable of eliciting a desired pharmacological response but which elicit unacceptable side effects at doses below that required for an effective amount of therapeutic response may be transformed by conjugation into a molecule useful in the treatment of an ACAMPS condition. Finally, trapping or accumulation of drug conjugates by phosphorylation may prevent the efflux of cancer drugs such as vinca alkaloids, epipodophyllotoxins, taxanes/taxoids, and anthracyclines, by the membrane transporter P-glycoprotein, thus, preventing a major form of MDR.

In certain embodiments, the substrate moiety in the conjugate may be any substrate for a kinase other than a hexokinase, a protein kinase or a lipid kinase that is overexpressed, overactive or that exhibits undesired activity in a target system. The action of the kinase on the substrate results in a modified conjugate wherein significant fraction of the activity of the drug moiety as well as the substrate moiety is retained. In a target system (e.g. cell, tissue or organ) containing cells the drug-substrate conjugate is less able to exit the cell in comparison to the unmodified drug. Accumulation of the drug-substratre conjugate into the target cells will occur by pushing the equilibrium of passive diffusion towards the target cells because of preferential trapping or accumulation due to the higher kinase activity in these cells.

In certain embodiments, the drug-substrate conjugates exhibit improved cytotoxic selectivity index over the parent drug. In certain embodiments, the drug-substrate conjugates exhibit improved solubility over the parent drug. In certain embodiments, the conjugates exhibit better serum stability than the parent drug. In certain embodiments, the conjugates exhibit better shelf life than the parent drug.

In one exemplary embodiment, the conjugates for use in the methods and compositions provided herein have the formula (1):
(D)_{d}-(L)_{q}-(S)ₜ (1)
or a pharmaceutically acceptable derivative thereof, wherein D is a drug moiety; d is 1-6, or is 1 or 2; L is a non-releasing linker; q is 0 to 6, or is 0 or 1; S is a substrate for a kinase other than a hexokinase, a protein kinase or a lipid kinase; and t is 1 to 6, or is 1 or 2, or is 1. In the conjugates, the drug moiety is covalently attached, optionally via a non-releasing linker, to the substrate.

In conjugates that contain two drug moieties, which are the same or different, conjugated to the substrate moiety(s) or non-releasing linked thereto can be at various positions of the substrate.

In certain embodiments, the conjugates have formula (2):
D-L-S, (2)
or a pharmaceutically acceptable derivative thereof, where the variables are as defined elsewhere herein.

Exemplary substrates, drug moieties, linkers and exemplary conjugates are described in further detail below. It is intended herein that conjugates resulting from all combinations and/or permutations of the groups recited below for the variables of formulae (1) and (2) are encompassed within the instant disclosure.

1. Drug Moiety

The conjugates provided herein are intended for modifying a variety of biological responses. The drug moiety may be any molecule, as well as a binding portion, fragment or derivative thereof that is capable of modulating a biological process other than compounds containing a carboranyl, hydroxyboryl or rare earth cryptate containing moiety. Thus, the drug moiety encompasses any molecule that elicits a pharmacological response that may be used for the treatment or prevention of a disease. Accordingly, the drug moities are any moities, including proteins and polypeptides, small molecules and other molecules that possess or potentiate a desired biological activity. Such molecules include cytotoxic agents, such as, but are not limited to, a toxin such as abrin, ricin A, pseudomonas exotoxin, shiga toxin, diphtheria toxin and other such toxins and toxic portions and/or subunits or chains thereof; proteins such as, but not limited to, tumor necrosis factor, α-interferon, γ-interferon, nerve growth factor, platelet derived growth factor, tissue plasminogen activator; or, biological response modifiers such as, for example, lymphokines, interleukin-I (IL-1), interleukin-2 (IL-2), interleukin-6 (IL-6), granulocyte macrophage colony stimulating factor (GMCSF), granulocyte colony stimulating factor (G-CSF), erythropoietin (EPO), pro-coagulants such as tissue factor and tissue factor variants, pro-apoptotic agents such FAS-ligand, fibroblast growth factors (FGF), nerve growth factor and other growth factors.

The drug moiety of the drug conjugate may be derived from a naturally occurring or synthetic compound that may be obtained from a wide variety of sources, including libraries of synthetic or natural compounds. For example, numerous means are available for random and directed synthesis of a wide variety of organic compounds and biomolecules. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs.

As such, the drug moiety may be obtained from a library of naturally occurring or synthetic molecules, including a library of compounds produced through combinatorial means (i.e., a compound diversity combinatorial library). When obtained from such libraries, the drug moiety employed will have demonstrated some desirable activity in an appropriate screening assay for the activity. Combinatorial libraries, as well as methods for the production and screening, are known in the art.

In particular embodiments, the drug moiety is a chemotherapeutic agent. Examples of chemotherapeutic agents include but are not limited to anti-infective agents, antihelminthic, antiprotozoal agents, antimalarial agents, antiamebic agents, antileiscmanial drugs, antitrichomonal agents, antitrypanosomal agents, sulfonamides, antimycobacterial drugs, or antiviral chemotherapeutics. Chemotherapeutic agents may also be antineoplastic agents or cytotoxic drugs, such as alkylating agents, plant alkaloids, antimetabolites, antibiotics, tubulin or microtubule binding agents and other anticellular proliferative agents.

Other specific drugs of interest include but are not limited to central nervous system depressants and stimulants, respiratory tract drugs, pharmacodynamic agents, such as histamines and antihistamines, cardiovascular drugs, blood and hemopoietic system drugs, gastrointestinal tract drugs, and locally acting drugs including chemotherapeutic agents. Drug compounds of interest from which drug moieties may be derived are also listed in: Goodman & Gilman's, The Pharmacological Basis of Therapeutics (9th Ed) (Goodman, et al., eds.) (McGraw-Hill) (1996); and 1999 Physician's Desk Reference (1998) and Chu, E.; DeVita, V. T. Physicians' Cancer Chemotherapy Drug Manual 2003, Jones and Bartlett Publishers.

Classes of cytotoxic agents for use herein include, for example, the a) anthracycline family of drugs, b) vinca alkaloid drugs, c) mitomycins, d) bleomycins, e) cytotoxic nucleosides, f) pteridine family of drugs, g) diynenes, h) estramustine, i) cyclophosphamide, j) taxanes, k) podophyllotoxins, l) maytansanoids, m) epothilones, and n) combretastatin and analogs.

In certain embodiments, the drug moiety is selected from a) doxorubicin, b) carminomycin, c) daunorubicin, d) aminopterin, e) methotrexate, f) methopterin, g) dichloromethotrexate, h) mitomycin C, i) porfiromycin, j) 5-fluorouracil, k) 6-mercaptopurine, l) cytosine arabinoside, m) podophyllotoxin, n) etoposide, o) etoposide phosphate, p) melphalan, q) vinblastine, r) vincristine, s) leurosidine, t) vindesine, u) estramustine, v) cisplatin, w) cyclophosphamide, x) paclitaxel y) leurositte, z) 4-desacetylvinblastine, aa) epothilone B, bb) taxotere, cc) maytansanol, dd) epothilone A, and ee) combretastatin and analogs. In certain embodiments, the drug is selected from Paclitaxel, Doxorubicin, Vinblastine, Methotrexate and Cisplatin.

Table 1 provides exemplary drug moieties used in the conjugates provided herein. Also indicated are points of attachment of the linker to the drug moieties and the functionality connecting the drug and the linker.

TABLE 1

| Structure of Drug/Drug Functional Group | Abbreviation |
|---|---|
| 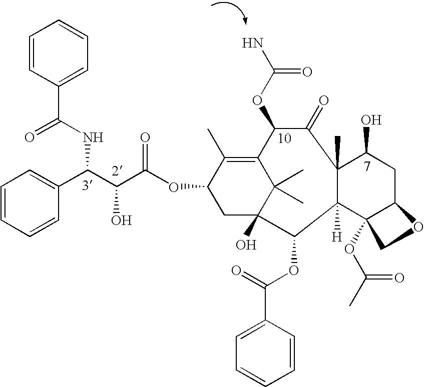 | 10Ca-PXL |
| 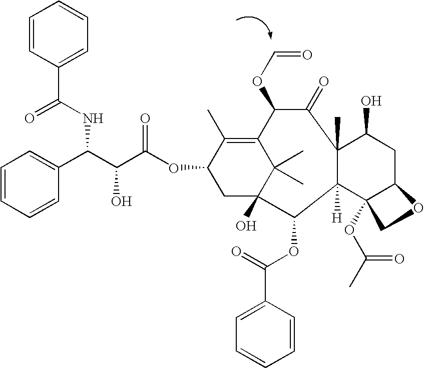 | 10Es-PXL |
| 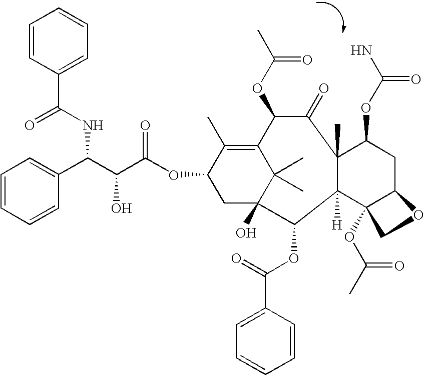 | 7Ca-PXL |
| 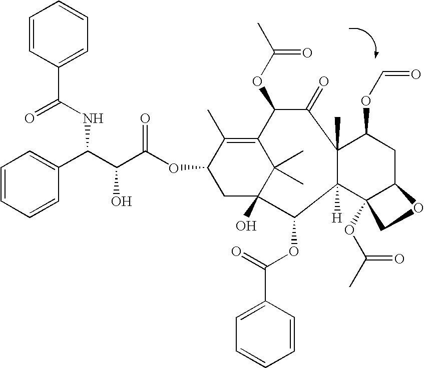 | 7Es-PXL |
| 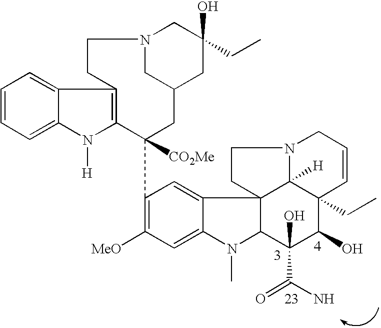 | 3Am-VBL |
| 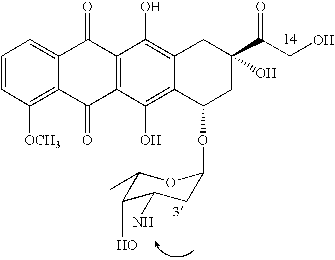 | 3′Alk-DOX |
| 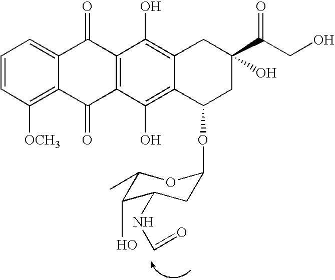 | 3′Am-DOX |

a) Arrows indicates site of attachment to drug (or functionality to drug) from Linker group of Table 2

Furthermore, other drug moieties that may have been tested and considered to have poor properties for treating cancer or proliferative disorders may also be used. When used in the conjugates provided herein, such drug moieties can exhibit enhanced biological activity compared to the unconjugated drug.

2. Linking Moiety

A linking moiety is used to attach the drug covalently to the substrate. The terms “linker” and “linking moiety” herein refer to any moiety that non-releasably connects the substrate moiety and drug moiety of the conjugate to one another. The linking moiety can be a covalent bond or a chemical functional group that directly connects the drug moiety to the substrate. The linking moiety can contain a series of covalently bonded atoms and their substituents which are collectively referred to as a linking group. Linking moieties are characterized by a first covalent bond or a chemical functional group that connects the drug moiety to a first end of the linker group and a second covalent bond or chemical functional group that connects the second end of the linker group to the substrate. The first and second functionality, which independently may or may not be present, and the linker group are collectively referred to as the linker moiety. The linker moiety is defined by the linking group, the first functionality if present and the second functionality if present. As used herein, the linker moiety contains atoms interposed between the drug moiety and substrate, independent of the source of these atoms and the reaction sequence used to synthesize the conjugate.

In one embodiment, the linker moiety is chosen to serve as a spacer between the drug and the substrate, to remove or relieve steric hindrance that may interfere with substrate activity and/or the pharmacological effect of the drug. The linker moiety can also be chosen based on its effect on the hydrophobicity of the drug-substrate conjugate, to improve passive diffusion into the target cells or tissue or to improve pharmacokinetic or pharmacodynamic properties. Thus, linking moieties of interest can vary widely depending on the nature of the drug and substrate moieties. In certain embodiments, the linking moiety is biologically inert. Precursors for a variety of linkers are known to those of skill in the art, which may be used in the synthesis of conjugates provided herein. Linker precursors are desirably synthetically accessible and provide shelf-stable products; and do not add any intrinsic biological activity that interferes with the conjugates activity. When incorporated into the conjugates, they can add desirable properties such as increasing solubility or stability to the conjugate.

Any bifunctional linker precursor, in certain embodiments, heterobifunctional linking precursors that can form a non-releasable bond between the drug moiety and the substrate moiety, when incorporated into the conjugates, can be used in the synthesis of conjugates provided herein. In certain embodiments, a linker precusor can be homobifunctional. In certain embodiments, one or more of substrate moieties are linked to one or more drug moieties via a multifunctional linking moiety.

In one embodiment, a linker precursor has functional groups that are used to interact with and form covalent bonds with functional groups in the components (e.g., drug moiety and substrate moiety) of the conjugates described and used herein. Examples of functional groups on the linker precursors (prior to interaction with other components) include —NH₂, —NHNH₂, —ONH₂, —NHC═(O)NHNH₂, —OH, —CHO, halogen, —CO₂H, and —SH. Each of these functional groups can form a covalent linkage to a suitable functional group on the substrate or the drug to get a drug-linker or a substrate-linker construct. For example, amino, hydroxy and hydrazino groups can each form a covalent bond with a reactive carboxyl group (e.g., a carboxylic acid chloride or activated ester such as an N-hydroxysuccinimide ester (NHS)). Other suitable bond forming groups are well-known in the art.

The linking moiety, L can include linear or acyclic portions, cyclic portions, aromatic rings or combinations thereof. In certain embodiments, the linking moiety can have from 1 to 100 main chain atoms other than hydrogen atoms, selected from C, N, O, S, P and Si. In certain embodiments the linking moiety contains up to 50 main chain atoms other than hydrogen, up to 40, up to 30, up to 20, up to 15, up to 10, up to 5, up to 2 main chain atoms other than hydrogen. In certain embodiments the linking moiety is acyclic.

In certain embodiments, the linking moieties contain oligomers of ethylene glycol or alkylene chains or mixtures thereof. These linking moieties are, in certain embodiments, attached to the substrate via either an alkyl or amide connection. In certain embodiments, the drug moiety is attached to the first end of the linker via an amide, sulfonamide, or ether connection. Illustrative synthetic schemes for forming such conjugates are discussed elsewhere herein for exemplary linkers for the conjugates provided herein.

In one embodiment, the linking moiety is a covalent bond between the drug moiety and the substrate moiety. Typically, this attachment is accomplished via coupling of a functional group on the drug with a compatible functional group on the substrate. In certain embodiments, the drug has an isocyanate, isothiocyanate or carboxylic acid functional group that is used to attach the drug to a hydroxy or amino group present on the substrate moiety to form a carbamate, thiocarbamate, urea or thiourea linkage between the components.

A variety of linking moieties depending on the nature of the drug and substrate moieties can be used in the conjugates provided herein. Suitable linking moieties can be selected by one of skill in the art based on the criteria set forth herein. In one embodiment, the linking moiety can be selected by the following procedure: A first end of a linker precursor is used in synthesizing a linker-nucleoside construct according to the procedures illustrated by Schemess 2, 3 and 6 and described herein. It is subjected to a first test which determines nucleoside kinase activity. In one embodiment, the method of the first test is by observing ADP formation which is an obligatory product of phospho group transfer from ATP using a coupled enzyme assay. ADP, formed from substrate phosphorylation (in conjugate form), is used by pyruvate kinase to generate pyruvate from phospoenolpyruvate which in turn is converted to lactate by lactate dehydrogenase. The lactate results in the consumption of NADH which is followed spectrophotometrically. The rate of substrate phosphorylation (in conjugate form) is then directly related to the rate of decrease in the observed NADH signal. By the aforementioned methods, a linker of appropriate length and a nucleoside or nucleoside analog is found with an effective amount of kinase activity which may be expected to be retained in the drug conjugate.

The linker found in the first test is subjected to a second test in certain embodiments, to determine suitability of the linker by connecting a second end of the linker precursor to a drug moiety. The site on the drug wherein the second end of the linker is attached is known to tolerate modification or may be shown to tolerate modification through a suitable functional group either pre-existing on the drug or on an analog thereof that is known to have an effective amount of the pharmacological activity of the parent drug. For example, paclitaxel modifications at C7, C10 and C3′-N are known to be tolerated as described in Kingston, Fortschr. Chem. Org. Naturst. 2002: 84, 53-225, the disclosure of which is incorporated by reference. In another example, camptothecin analogs with suitable functionalities for linker attachment are described in Wall, et. al., J. Med. Chem. 1993: 36, 2689-2700 whose disclosure is incorporated by reference.

In another embodiment, conjugates based upon Vinblastine are prepared by use of the natural product O4-deacetyl Vinblastine or Vindesine prepared according to Barnett, et. al. J. Med. Chem. 1978: 21, 88-96, whose disclosure is incorporated by reference. Vindesine and O⁴-deacetyl Vinblastine are characterized by a free hydroxyl group at C4. Alternatively, vinblastine conjugates are prepared from β4-deacetyl-3-de-(methoxycarbonyl)-vinblastin-3-yl carbonyl azide through condensation with amines as described in Lavielle, et. al. J. Med. Chem. 1991: 34, 1998-2003, the disclosure of which is incorporated by reference. A second test of a drug-linker construct may then be determined by a functional assay which is predictive of pharmacological activity. For example, microtubule stabilization for paclitaxel drug linker constructs or microtubule disruption by vinblastine drug-linker constructs is determined with a tubulin polymerization assay as described in Barron et. al. Anal. Biochem. 2003: 315, 49-56 the disclosure of which is incorporated by reference.

Tubulin assembly or inhibition thereof can be monitored by fluorescence using the CytoDYNAMIX Screen™ 10 kit available from Cytoskeleton (1830 S. Acoma St., Denver, Colo.). The kit is based upon an increase in quantum yield of florescence upon binding of a fluorophore to tubulin and microtubules and a 10× difference in affinity for microtubules compared to tubulin. Compounds such as paclitaxel which enhance tubulin assembly will therefore give an increase in emission whereas compounds such as vinblastine which inhibit tubulin assembly will give a decrease in emission. Tubulin assembly or inhibition thereof can also be monitored by light scattering which is approximated by the apparent absorption at 350 nm.

In certain embodiments, doxorubicin-linker constructs can be screened by monitoring alteration in the ability of Topoisomerase II to catalyze the formation of relaxed conformation DNA from a super-coiled plasmid.

In another embodiment, a functional assay for camptothecin drug-linker constructs depends on Topoisomerase I binding to DNA an example of which is given in Demarquay, Anti-Cancer Drugs 2001: 12, 9-19 the disclosure of which is incorporated by reference. It should be appreciated that an appropriate linker may also be found by interchanging the order of the first and second tests.

In one embodiment, the linking moiety in the conjugates provided herein contains an alkylene chain containing from 1 up to 50 main chain atoms other than hydrogen. In certain embodiments, the alkylene chain contains 2, 3, 4, 5, 6, 7, 8, 9, 10 or 15 main chain atoms other than hydrogen. In other embodiments, the alkylene chain contains 3, 4, 5, 6, 7, 8 or 9 main chain atoms other than hydrogen.

In other embodiment, the linking moiety in the conjugates provided herein contains a polyethylene glycol (PEG) chain. The PEGs for use herein can contain up to 50 main chain atoms other than hydrogen. In certain embodiments, the PEG contains 5, 11, 13, 14, 22 or 29 main chain atoms other than hydrogen. In certain embodiments, the PEG contains 5, 11, 13 or 29 main chain atoms other than hydrogen. In other embodiment, the linker moiety contains a combination of alkylene, PEG and maleimide units in the chain. Some exemplary linking groups incorporated into the conjuagates are provided in Table 2. As exemplified in Table 2, the linking groups are named based on the chemical units present and the number of main atoms, other than hydrogen are indicated in the parenthesis.

TABLE 2

| Structure of Linker Groups | Abbreviation |
|---|---|
| 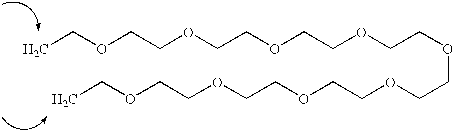 | PEG(29) |
| 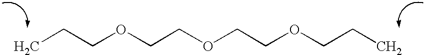 | PEG(13) |
| 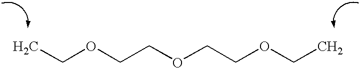 | PEG(11) |
| 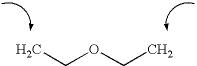 | PEG(5) |
| 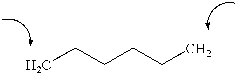 | ALK(6) |
| 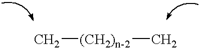 | ALK(n) |
| 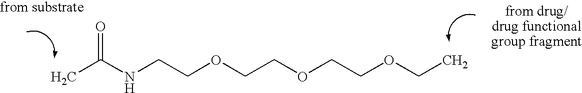 | PEGa(14) |
| 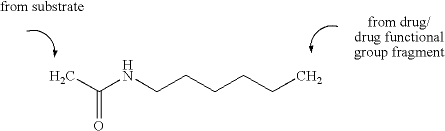 | ALKa(9) |
| 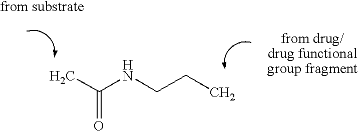 | ALKa(6) |
| 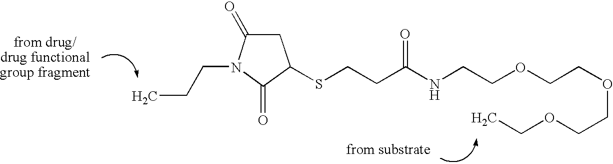 | [MALaPEG](22) |
| 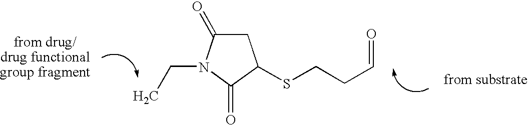 | MAL(8) |
| 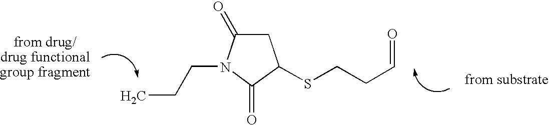 | MAL(9) |

a) Arrows indicates site of attachment to drug (or functionality to drug) and to substrate (or functionality to substrate). For unsymmetrical linker groups directionality of attachment to drug and substrate is so indicated

Several linker precursors useful in the conjugates provide herein are described in U.S. Pat. Nos. 5,512,667; 5,451,463; and 5,141,813. In addition, U.S. Pat. Nos. 5,696,251; 5,585,422; and 6,031,091 describe certain tetrafunctional linking groups that can be used for the conjugates provided herein.

3. Substrates

The substrate moiety may be any substrate for a kinase that is overexpressed, overactive or that exhibits undesired activity in a target system, wherein the kinase is other than a hexokinase, a protein kinase or a lipid kinase. In certain embodiments, the substrate has a molecular weight between about 50 amu and 1000 amu. The kinase is present at a higher concentration or operates at a higher activity, or the activity is undesired or persistent in a cell type that contributes to the genesis or maintenance of the condition being treated in the target cell in comparison to other cells. Addition of a phosphate group by action of the kinase on the substrate confers a negative charge to the conjugate, thus trapping or accumulating the conjugate inside the targeted cells at concentrations higher than will be achieved in other cells not involved with the condition being treated.

The action of the kinase on the substrate results in a modified conjugate in the target system (e.g. cell, tissue, organ), which is less able to exit the target system in comparison to the unmodified conjugate. In another embodiment, the kinase is associated with an ACAMPS-related condition.

In certain embodiments, the substrate is a substrate for a kinase such as a nucleoside kinase. In certain embodiments, the substrate is a substrate for a kinase such as thymidine kinase, viral thymidine kinase, human thymidine kinase TK-1, deoxycytidine kinase, deoxyguanosine kinase. In other embodiments, the substrate is a substrate for viral thymidine kinase, human thymidine kinase TK-1.

In certain embodiments, the substrate is selected from nucleosides and their natural and non-natural analogs. Examples of nucleosides for use as substrates in the conjugates herein, but are not limited to, cytidine, uridine, thymidine, guanosine, adenosine, or derivatives thereof. In one embodiment, the substrate is a nucleoside or nucleoside analog for thymidine kinase, viral thymidine kinase, TK-1, deoxycytidine kinase or deoxyguanosine kinase known or found to be activated in cells associated with ACAMPS-related conditions. Natural and non-natural nucleoside analogs are contemplated herein. In another embodiment, the substrate is a nucleoside or nucleoside base which is converted to a substrate of thymidine kinase, viral thymidine kinase, TK-1 or deoxycytidine kinase by the action of thymidine phosphorylase or cytidine deaminase.

Table 3 shows illustrative examples of known pyrimidine nucleoside analogs which are substrates for thymidine kinase, and deoxycytidine kinase; and purine analogs which are substrates for deoxycytidine kinase and deoxyguanosine kinase, for use in the conjugates and methods provided herein. (Johansson et al., Acta Biochim. Polonica 43: 143-160 (1996); Eriksson et al., Biochem. Biophys. Res. Commun. 176: 586-592 (1991); Wang et al., Biochemistry 38: 16993-16999 (1999)). Deoxycytosine kinase shows low enantioselectivity for 2′-deoxycytidine and analogs thereof (Verri, A. et al. Mol. Pharm. 51: 132-138 (1997)) and deoxyguanosine kinase and deoxycytidine kinase show low enantioselectivities for 2′deoxyadenosine, 2′deoxyguanosine and analogs thereof (Gaubert, G., et al. Biochimie 81: 1041-1047 (1999)). Therefore, D- and L-pyrimidine and purine nucleoside analogs are contemplated substrates.

Other contemplated substrates include pyrimidine analogs covalently linked to a non-deoxy-ribose sugar having an anomeric carbon (α and β anomers). Such substrates include but are not limited to β-L-2′,3′-dideoxy-3′-thiacytidine (3TC), β-L-1,3-dioxolane-cytidine (L-OddC), (North)-methanocarba-thymidine and analogs thereof. Still other contemplated substrates are acyclic and carbocyclic analogs of guanosine which are known in the art as substrates for viral thymidine kinase. (For a review see De Clerq, D. E. et al., Nucleosides, Nucleotides & Nucleic Acids 20: 271-285 (2001)).

TABLE 3
Johansson et al., supra

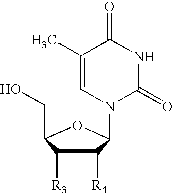

| | TK1 | TK2 | dCK |
|---|---|---|---|
| R4 = H, R3 = OH (Thy) | 100 | 100 | 2 |
| R4 = H, R3 = F (FLT) | 30 | | |
| R4 = H, R3 = N3 (AZT) | 50 | 5 | |
| R4 = H, R3 = CH2N3 | 15 | 3 | |
| R4 = H, R3 = CCH | <1 | <1 | |
| BBCR 1991; 176, 586 | | | |
| R4 = OH, R3 = OH | | | |
| (RiboThymidine) | 2 | 3 | |
| R4 = H, R3 = H | 40 | 4 | <1 |
| R4 = H, R3 = F (FLT) | 30 | <1 | <1 |
| R4 = H, R3 = N3 (AZT) | 40 | 5 | <1 |

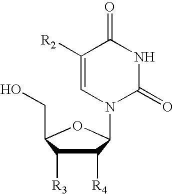

| | | | |
|---|---|---|---|
| R2 = H, R4 = H, R3 = OH (dU) | 100 | 100 | 6 |
| R2 = CH3, R4 = H, R3 = OH (Thy) | 100 | 100 | 2 |
| R2 = H, R4 = H, R3 = H (ddU) | 10 | 2 | <1 |
| R2 = F, R4 = H, R3 = OH | 90 | 90 | |
| R2 = Br, R4 = H, R3 = OH | 80 | 10 | <1 |
| R2 = NH2, R4 = H, R3 = OH | 3 | 50 | 17 |
| R2 = Et, R4 = H, R3 = OH | 80 | 10 | |
| R2 = —CH₂═CH₂CH₃, R4 = H, R3 = OH | <1 | 40 | |

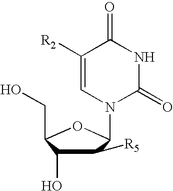

| | TK1 | TK2 |
|---|---|---|
| R2 = CH3, R5 = F (FMAU) | 45 | 100 |
| R2 = I, R5 = F (FIAU) | 42 | 90 |
| R2 = CH3, R5 = OH (araT) | | 60 |
| R2 = H, R5 = OH (araU) | | 20 |
| R2 = cyclopropyl, R5 = OH | | 15 |
| 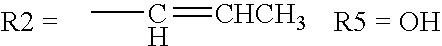 | | 50 |
| 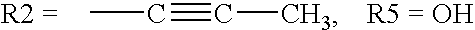 | | 30 |
| R2 = 2-thienyl, R5 = OH | | 6 |

Eriksson et al., supra

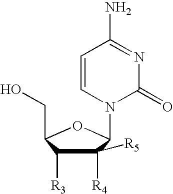

| | TK2 | dCK |
|---|---|---|
| R4 = H, R5 = H, R3 = OH (dC) | 90 | 100 |
| R4 = H, R5 = OH, R3 = OH | <1 | 120 |
| (Cytosar, AraC) | | |
| R4 = OH, R5 = H, R3 = OH | <1 | 20 |
| R4 = H, R5 = H, R3 = H (ddCy, | <1 | 30 |
| Zalcitabine) | | |
| R4 = F, R5 = H, R3 = OH | 30 | 300 |
| R4 = H, R5 = H, R3 = F | <1 | 60 |
| 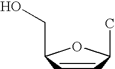 | <1 | 4 |
| 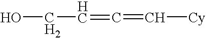 | <1 | 20 |
| (±)-Cytallene | | |

Johansson et al., supra

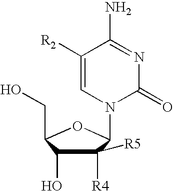

| | TK1 | TK2 | dCK |
|---|---|---|---|
| R4 = H, R5 = OH, R2 = H | <1 | <1 | 120 |
| (Cytosar, AraC) | | | |
| R4 = H, R5 = F, R2 = H | | | 110 |
| R4 = H, R5 = F, R2 = F | | | 110 |
| R4 = H, R5 = F, R2 = 2-thienyl | <1 | 100 | 10 |
| R4 = OH, R5 = H, R2 = H | <1 | <1 | 20 |
| (Cytosine) | | | |
| R4 = H, R5 = H, R2 = H (dC) | <1 | 90 | 100 |
| R4 = N3, R5 = H, R2 = H | | | 20 |
| R4 = F, R5 = H, R2 = H | <1 | 30 | 300 |
| R4 = OCH3, R5 = H, R2 = H | | | 80 |

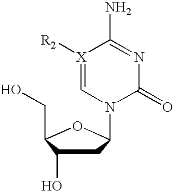

| | TK2 | dCK |
|---|---|---|
| R2 = H, X = C (dC) | 90 | 100 |
| R2 = CH3, X = C | 40 | 60 |
| R2 = cyclopropyl, X = C | <1 | 20 |
| R2 = Br, X = C | 40 | 20 |
| R2 = F, X = C | 90 | 20 |
| X = N (5-aza) | | 20 |
| R2 = 2-thienyl, X = C | 30 | 2 |
| R2 = 3-thienyl, X = C | 30 | <1 |
| R2 = 2-furyl, X = C | 10 | 3 |
| R2 = 3-furyl, X = C | 50 | <1 |
| R2 = 2-pyridyl, X = C | 7 | <1 |
| R2 = 3-pyridyl, X = C | <1 | 4 |
| R2 = 4-pyridyl, X = C | 2 | 5 |
| Wang et al., supra | | |
| β D-Thy | 100 | |
| β-L-Thy | 60 | 0.8 |
| α-D-Thy | 2.4 | |
| α-L-Thy | 1.5 | |
| β-D-dC | 93 | 100 |
| β-L-dC | 70 | 40 |
| α-D-dC | 1.8 | 1.8 |
| α-L-dC | 0.4 | |
| β-ddT(3′-deoxyThy) | 3.2 | |
| α-ddT | 9.8 | |
| β-ddC(2′,3′-dideoxyC) | | 7.1 |
| α-ddC | | 29 |

Johansson et al., supra

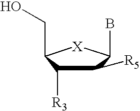

| R | dCK | dGK |
|---|---|---|
| X = O, R3 = OH, R5 = H, B = Adenine (dado) | 350 | 100 |
| X = O, R3 = OH, R5 = H, B = Guanine (dGuo) | 300 | 50 |
| X = O, R3 = OH, R5 = H, B = Hypoxanthine (dino) | 120 | 100 |
| X = O, R3 = OH, R5 = H, B = 7-deaza-Adenine | 50 | 100 |
| X = O, R3 = OH, R5 = H, B = 2-Cl-Adenine | 260 | 180 |
| X = O, R3 = CH2OH, R5 = H, B = 2-Cl-Adenine | 40 | |
| X = O, R3 = OH, R5 = OH, B = Adenine (AraA) | 50 | 15 |
| X = O, R3 = OH, R5 = OH, B = Guanine (AraG) | 6 | 180 |
| X = O, R3 = OH, R5 = OH, B = Hypoxanthine | | 290 |
| X = CH2, R3 = OH, R5 = H, B = Guanine | | 70 |

In certain embodiments, the substrate is a nucleoside or nucleoside analog substrate for a thymidine kinase (TK). The TK is active within a cell type that contributes to the genesis or maintenance of a disease. Phosphorylation of the nucleoside or nucleoside analog by the TK leads to trapping or accumulation of the conjugate within the targeted cell type due to the drug-conjugate acquiring a negative charge. Due to failure to introduce the foreign gene into every cancer cell, previous efforts with gene therapy to effect targeting of a nucleoside prodrug to tumors by introducing a foreign TK into the cancer cell has not lead to clinical success (for review see Fillat, et. al. Curr. Gene Ther. 2003: 3: 13-26).

In another embodiment, the substrate is a nucleoside or nucleoside analog substrate for human thymidine TK-1 or a viral TK. The drug-nucleoside or drug-nucleoside analog conjugate, in one embodiment, is effective in treating cancer through phosphorylation of the drug-nucleoside or drug-nucleoside analog conjugate by TK-1, leading in certain embodiment, to trapping or accumulation of the conjugate and hence the anti-cancer agent within the cancer cell. Therefore, trapping or accumulation is responsible for the therapeutic effect of these conjugates in the treatment of cancer. The therapeutic effect is due to the accumulated anti-cancer drug which is active in the conjugate and is not due to the nucleoside or nucleoside analog, which simply serves as a substrate for the targeting enzyme. Furthermore, the therapeutic effect of the drug conjugate is not dependent on release of free drug. In one embodiment, no further intervention of intracellular proteins is required for activation of the drug within the conjugate. Further action by thymidylate kinase and incorporation into DNA is not precluded as an additional enhancement of the therapeutic effect of the drug conjugate in the treatment of cancer.

In certain embodiments, the drug moiety and/or the substrate moiety in the conjugate can be present in a form of a pharmaceutically acceptable derivative that renders the conjugate biologically inactive. The inactive drug-substrate conjugate can be converted to the active drug-substrate conjugate under physiological conditions or by intracellular proteins without having the need to cleave the drug-substrate conjugate.

The anti-cancer drug-nucleoside conjugates are effective in treating viral infections, such as DNA or RNA viral infections, by using a viral TK which results in trapping or accumulation of a drug which is responsible for the therapeutic effect of these conjugates. A cell infected with a RNA or DNA virus is distinguished by a TK activity introduced by the virus into the cell. The viruses include, but are not limited to, HSV-1, HSV-2, VZV, EBV, CMV, HTLV-1 and HIV. A thymidine kinase that is aberrant in a cell type involved in the genesis or maintenance of the condition is used to target the cell types with a conjugate of a drug to a nucleoside or nucleoside analog that is a substrate of the kinase. Addition of a phosphate group by action of the thymidine kinase on the nucleoside or nucleoside analog confers a negative charge to the conjugate thus trapping or accumulating the drug inside the targeted cells at concentrations higher than will be achieved in other cells not involved with the condition being treated. The therapeutic effect is due to the accumulated drug which is active in the conjugate and is not due to the nucleoside which simply serves as a substrate for the targeted enzyme or release of free drug. Therefore, as discussed above, no further intervention of intracellular proteins is required for activation of the drug within the conjugate though cleavage of the linker to give free drug. In certain embodiments, where the conjugate is present as a pharmaceutically acceptable derivative of the conjugates provided herein, the intracellular proteins may activate the conjugate by converting the conjugate in the active form. The nucleoside conferring an additional therapeutic effect is not precluded. Further action by thymidylate kinase and incorporation into DNA is not precluded as an additional enhancement of the therapeutic effect of the drug conjugate in the treatment of viral infections.

Thymidylate synthase (TS) and thymidine kinase (TK) are the key enzymes in the synthesis of pyrimidine nucleotides required for cell division. In the de novo pathway TS catalyzes the reductive methylation of dUMP to dTMP. In the salvage pathway TK directly catalyzes the phosphorylation of thymidine released from cells by DNA catabolism. Both enzymes are highly expressed in breast, gastric, ovarian, colorectal and bladder carcinomas to name a few. There are already a large number of anti-metabolite drugs that target TS, notably 5-fluorouracil (5-FU). However, patients treated with 5-FU rapidly develop resistance, resulting from increased expression of TS, TK or both. Of the two known human TK isozymes, TK1 and TK2, TK1 is preferentially up-regulated in carcinomas. Some virus encoded TKs have been shown to differ in substrate specificity from the corresponding TK isozymes in the host cells (for review see Hannigan, et. al. Cancer Biother. 1993: 8, 189-97).

In another embodiment, the substrate is a substrate for deoxycytidine kinase, including, but not limited to, cytidine and uridine derivatives. Deoxycytidine kinase (dCK) is known to phosphorylate cytostatic drugs (e.g., gemcitabine) for activation. It is contemplated that dCK may have greater tolerance towards uridine derivatives, in comparison to TK. For example, 5-amino-uridine which has been shown to have a 17% activity towards dCK may be used as a substrate, utilizing the amino group as a potential site for linkage to the conjugate. Furthermore, dCK is known to have greater tolerance towards carbohydrate modifications than thymidine kinase. For example, dCK shows only modest discrimination between the natural nucleoside β-D-cytidine and its enantiomer β-L-cytidine. Furthermore, stereochemistry at C—I is not critical for recognition since α-anomers are also accepted as substrates (see Wang, J. et al. Biochemistry 1999, 38: 16993 and Verri, A. et al. Mol. Pharm. 1997, 51: 132.) Contemplated substrates include, but are not limited to, cytostatic nucleosides known in the art to be substrates for TK and/or dCK and anti-viral nucleosides known in the art to be substrates for viral thymidine kinase. Additional contemplated substrates include but are not limited to β-D-, β-L-, α-D-, and α-L-nucleoside analogs and acyclic carbohydrate analogs, which may also utilize the acyclic carbohydrate as a potential site for linkage to the conjugate.

4. Exemplary Conjugates

In certain embodiments, the conjugates provided herein contain a substrate that is a substrate for a nucleoside kinase and the conjugates have formula:
N-L-D
or a pharmaceutically acceptable derivative thereof, wherein N is a natural or non-natural nucleoside; L, which may or may not be present, is a non-releasing linker and D is a drug moiety. The drug is non-releasably linked to a carbohydrate or a base moiety of the nucleoside. In certain embodiments, the drug is linked to the carbohydrate moiety of the nucleoside. In other embodiments, the drug is linked to the base of the nucleoside moiety of the nucleoside.

In certain embodiments, the conjugates have a formula:
S_{c}-P¹-L-D,
or a pharmaceutically acceptable derivative thereof, wherein S_{c} is ribose, deoxyribose or analog thereof; P¹ is a purine, pyrimidine or analog thereof and other variables are as defined herein.

In certain embodiments, the conjugates have a formula:
P¹-S_{c}-L-D,
or a pharmaceutically acceptable derivative thereof, wherein S_{c} is ribose, deoxyribose or analog thereof; P¹ is a purine, pyrimidine or analog thereof and other variables are as defined herein.

In certain embodiments, the conjugates provided herein have formula: 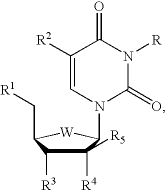
or a pharmaceutically acceptable derivative thereof,
wherein
R¹, R³, R⁴ and R⁵ are each independently Y, H, hydroxy, halo, azido, C1-6 alkyl and optionally containing a heteroatom, C2-6 alkenyl or C2-6 alkynyl;
R² is Y, H, C1-6 alkyl and optionally containing a heteroatom, C2-6 alkenyl, C2-6 alkynyl, C3-6 cycloalkyl, aryl or heteroaryl;
R is Y, H or C1-6 alkyl, C2-6 alkenyl or C2-6 alkynyl;
W is CR^{e}R^{f} or O; R^{e} and R^{f} are each independently H or C1-6 alkyl;
Y is a linker-drug construct L-D, wherein L, which may or may not be present, is a non-releasing linker and D a drug moiety;
R, R¹ and R³-R⁵ are selected such that at least one of R, R¹ and R³-R⁵ is Y and at least one of R, R¹ and R³-R⁵ is OH;
R¹-R⁵ and R are unsubstituted or substituted with one or more substituents, in one embodiment 1-4 substituents, in another embodiment 1 or 2 substituents, each independently selected from Q¹.

In certain embodiments, Q¹ is halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N+R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q¹ groups, which substitute atoms in a 1, 2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹ groups, which substitute the same atom, together form alkylene; and
each Q¹ is independently unsubstituted or substituted with one or more substituents, in one embodiment one, two or three substituents, each independently selected from Q²;
each Q² is independently halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1, 2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;
R⁵⁰ is hydroxy, alkoxy, aralkoxy, alkyl, heteroaryl, heterocyclyl, aryl or —NR⁷⁰R⁷¹, where R⁷⁰ and R⁷¹ are each independently hydrogen, alkyl, aralkyl, aryl, heteroaryl, heteroaralkyl or heterocyclyl, or R⁷⁰ and R⁷¹ together form alkylene, azaalkylene, oxaalkylene or thiaalkylene;
R⁵¹, R⁵² and R⁵³ are each independently hydrogen, alkyl, aryl, aralkyl, heteroaryl, heteroaralkyl, heterocyclyl or heterocyclylalkyl;
R⁶⁰ is hydrogen, alkyl, aryl, aralkyl, heteroaryl, heteroaralkyl, heterocyclyl or heterocyclylalkyl; and

R⁶³ is alkoxy, aralkoxy, alkyl, heteroaryl, heterocyclyl, aryl or —NR⁷⁰R⁷¹.

In certain embodiments, R¹ is, H, hydroxy, halo, azido, C1-6 alkyl and optionally containing a heteroatom, C2-6 alkenyl or C2-6 alkynyl. In one embodiment, R¹ is OH.

In certain embodiments, R³ is Y, H, hydroxy, halo, azido, C1-6 alkyl and optionally containing a heteroatom, C2-6 alkenyl or C2-6 alkynyl. In other embodiments, R³ is hydroxy. In other embodiments, R³ is Y.

In certain embodiments, R⁴ is Y, H, hydroxy, halo, azido, C1-6 alkyl and optionally containing a heteroatom, C2-6 alkenyl or C2-6 alkynyl. In other embodiments, R⁴ is hydroxy. In other embodiments, R⁴ is H. In other embodiments, R⁴ is Y.

In certain embodiments, R⁵ is Y, H, hydroxy, halo, azido, C1-6 alkyl and optionally containing a heteroatom, C2-6 alkenyl or C2-6 alkynyl. In other embodiments, R⁵ is H. In other embodiments, R⁵ is Y.

In certain embodiments, R² is Y, H, C1-6 alkyl and optionally containing a heteroatom, C2-6 alkenyl, C2-6 alkynyl, C3-6 cycloalkyl, aryl or heteroaryl. In other embodiments, R² is H. In other embodiments, R² is C1-6 alkyl. In other embodiments, R² is methyl. In other embodiments, R² is Y.

In certain embodiments, R is Y, H or C1-6 alkyl. In one embodiment, R is H. In another embodiment, R is C1-6 alkyl. In other embodiments, R is Y.

In certain embodiments, W is CR^{e}R^{f} or O. In certain embodiments, W is O. In certain embodiments, W is CR^{e}R^{f}. In other embodiments, R^{e} and R^{f} are each H.

In certain embodiments, Y is -L-D, where L is a non-releasing linker and D is a drug moiety. In other embodiments, Y is D. In certain embodiments, -L- is —O-L₁-, where L₁ is non-releasing linker. In other embodiments, -L₁- is selected from a mono or bifunctional alkelene chain or mono or bifunctional polyethylene glycol chain.

In certain embodiments, the conjugates have formula: 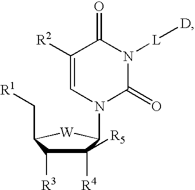
or a pharmaceutically acceptable derivative thereof,
wherein, R¹ and R³ are Hydroxy; R⁴ is H or F; R⁵ is H, OH or F; R² is H, C1-6 alkyl or halo, W is O and other variables are as described herein.

In certain embodiments, the conjugates have formula: 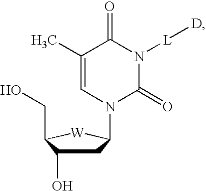
or a pharmaceutically acceptable derivative thereof,
wherein, L is a non-releasing linker and D a drug moiety.

In another embodiment, the conjugates have formula: 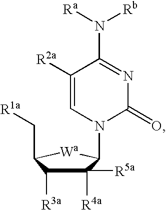
or a pharmaceutically acceptable derivative thereof,
wherein
R^{1a}, R^{3a}, R^{4a} and R^{5a} are each independently Y; H, hydroxy, halo, azido, C1-6 alkyl and optionally containing a heteroatom, C2-6 alkenyl or C2-6 alkynyl;
R^{2a} is Y, H, C1-6 alkyl and optionally containing a heteroatom, C2-6 alkenyl, C2-6 alkynyl, C3-6 cycloalkyl, hydroxy, aryl or heteroaryl or halo;
Y is a linker-drug construct L-D, wherein L, which may or may not be present, is a non-releasing linker and D is a drug moiety;
R^{a} and R^{b} are each independently Y, H, or C1-6 alkyl;
R^{d} is H or C1-6 alkyl;
W^{a} is CR^{e}R^{f} or O; R^{e} and R^{f} are each independently H or C1-6 alkyl;
R^{1a}-R^{5a}, R^{a} and R^{b} are selected such that at least one of R^{1a}-R^{5a}, R^{a} and R^{b} is Y and at least one of R^{1a}, R^{3a}-R^{5a} is OH;
R^{1a}-R^{5a}, R^{a}, R^{b} and R^{d} are unsubstituted or substituted with one or more substituents, in one embodiment 1-4 substituents, in another embodiment 1 or 2 substituents, selected from Q¹.

In certain embodiments, R^{1a}, R^{3a}, R^{4a} and R^{5a} are each independently Y, H, hydroxy, halo, azido, C1-6 alkyl and optionally containing a heteroatom, C2-6 alkenyl or C2-6 alkynyl. In certain embodiments, R^{1a} is Y, H, hydroxy, halo, azido, C1-6 alkyl and optionally containing a heteroatom, C2-6 alkenyl or C2-6 alkynyl. In certain embodiments, R^{1a} is Y or hydroxy. In certain embodiments, R^{1a} is Y. In certain embodiments, R^{1a} is hydroxy. In certain embodiments, R^{3a} is Y or hydroxy, In certain embodiments, R^{3a} is Y. In certain embodiments, R^{3a} is hydroxy. In certain embodiments, R^{4a} is Y or hydroxy. In certain embodiments, R^{4a} is Y. In certain embodiments, R^{4a} hydroxy. In certain embodiments, R^{5a} is Y or hydroxy. In certain embodiments, R^{5a} is Y. In certain embodiments, R^{5a} is H.

In one embodiment, R^{2a} is Y, H or C1-6 alkyl. In other embodiments, R^{2a} is Y. In other embodiments, R^{2a} is H.

In other embodiment, R^{a} and R^{b} are each independently Y, H or C1-6 alkyl. In other embodiment, R^{a} and R^{b} are each independently H.

In other embodiment, W^{a} is O.

In certain embodiments, Y is -L-D, where L is a non-releasing linker and D is a drug moiety. In other embodiments, Y is D. In certain embodiments, -L- is —O-(L₁)_{q}—, where L₁ is non-releasing linker and q is 0-2. In other embodiments, -L₁- is selected from a mono or bifunctional alkelene chain or mono or bifunctional polyethylene glycol chain.

In another embodiment, the conjugates have formula: 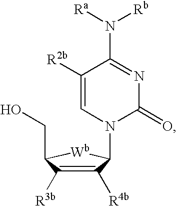
or a pharmaceutically acceptable derivative thereof, wherein
R^{3b} and R^{4b} are each independently Y, H, C1-6 alkyl and optionally containing a heteroatom, C2-6 alkenyl or C2-6 alkynyl;
R^{2b} is Y, H, C1-6 alkyl and optionally containing a heteroatom, C2-6 alkenyl, C2-6 alkynyl, C3-6 cycloalkyl, aryl, heteroaryl or halo;
Y is a linker-drug construct L-D, wherein L, which may or may not be present, is a non-releasing linker and D is a drug moiety;
W^{b} is CR^{e}R^{f} or O; R^{e} and R^{f} are each independently H or C1-6 alkyl;
R^{a} and R^{b} are independently Y, H, or C1-6 alkyl;
R^{d} is H or C1-6 alkyl;
R^{1b}-R^{4b}, R^{a} and R^{b} are selected such that at least one of R^{1b}-R^{4b}, R^{a} and R^{b} is Y;
R^{1b}-R^{4b}, R^{a}, R^{b} and R^{d} are unsubstituted or substituted with one or more substituents, in one embodiment 1-4 substituents, in another embodiment 1 or 2 substituents, selected from Q¹.

In other embodiments, R^{2b} is Y or H. In other embodiments, R^{2b} is Y.

In another embodiment, the conjugates have formula: 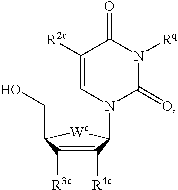
or a pharmaceutically acceptable derivative thereof, wherein
R^{3c} and R^{4c} are each independently Y, H, C1-6 alkyl and optionally containing a heteroatom, C2-6 alkenyl or C2-6 alkynyl;
R^{2c} is Y, H, C1-6 alkyl and optionally containing a heteroatom, C2-6 alkenyl, C2-6 alkynyl, C3-6 cycloalkyl, hydroxy, aryl, heteroaryl, or halo;
R^{q} is Y, H or C1-6 alkyl;
Y is a linker-drug construct L-D, wherein L, which may or may not be present, is a non-releasing linker and D is a drug moiety;
W^{c} is CR^{e}R^{f} or O; R^{e} and R^{f} are each independently H or C1-6 alkyl;
R^{1c}-R^{4c} and R^{q} are selected such that at least one of R¹-R^{4c} or R^{q} is Y;
R^{1c}-R^{4c} and R^{q} are unsubstituted or substituted with one or more substituents, in one embodiment 1-4 substituents, in another embodiment 1 or 2 substituents, selected from Q¹.

In certain embodiments, R^{1c} is Y. In certain embodiments, R^{2c} is Y. In certain embodiments, R^{q} is Y.

In another embodiment, the conjugates have formula: 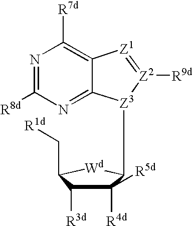
or a pharmaceutically acceptable derivative thereof, wherein
R^{1d}, R^{3d}, R^{4d} and R^{5d} are each independently Y, H, hydroxy, halo, azido, C1-6 alkyl and optionally containing a heteroatom, C2-6 alkenyl or C2-6 alkynyl;
R^{7d} is Y, H, hydroxy, halo, azido, C1-6 alkyl and optionally containing a heteroatom, C2-6 alkenyl, C2-6 alkynyl, thioalkyl or NR^{a}R^{b};
R^{8d} is Y, H, alkyl, halo, SR^{d} or NR^{a}R^{b};
R^{9d} is Y, H, or C1-6 alkyl;
Y is a linker-drug construct L-D, wherein L, which may or may not be present, is a non-releasing linker and D is a drug moiety;
R^{1d}-R^{9d} are selected such that at least one of R^{1d}, R^{3d}, R^{4d}, R^{5d} or R^{7d} is Y and at least one of R^{1d}, R^{3d}, R^{4d}, R^{5d} or R^{7d} is OH;
R^{a} and R^{b} are each independently Y, H, or C1-6 alkyl;
R^{d} is H or C1-6 alkyl;
W^{d} is CR^{e}R^{f} or O; R^{e} and R^{f} are each independently H or C1-6 alkyl;
Z¹, Z² and Z³ are each independently C or N;
R^{1d}-R^{9d}, R^{a}, R^{b} and R^{d} are unsubstituted or substituted with one or more substituents, in one embodiment 14 substituents, in another embodiment 1 or 2 substituents, selected from Q¹.

In another embodiment, the conjugates have formula: 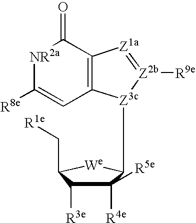
or a pharmaceutically acceptable derivative thereof, wherein R^{1e}, R^{3e}, R^{4e} and R^{5e} are each independently Y, H, hydroxy, halo, azido, C1-6 alkyl and optionally containing a heteroatom, C2-6 alkenyl or C2-6 alkynyl;
R^{2e} is Y, H, C1-6 alkyl and optionally containing a heteroatom, C2-6 alkenyl, C2-6 alkynyl or C3-6 cycloalkyl;
R^{8e} is Y, H, alkyl, halo, SR^{d} or NR^{a}R^{b};
R^{9e} is Y, H, or C1-6 alkyl;
Y is a linker-drug construct L-D, wherein L, which may or may not be present, is a non-releasing linker and D is a drug moiety;
R^{1e}-R^{5e}, R^{8e} and R^{9e} are selected such that at least one of R^{1e}-R^{5e}, R^{8e} and R^{9e} is Y and at least one of R^{1e}, R^{3e}, R^{4e} and R^{5e} is OH;
we is CR^{e}R^{f} or O; R^{e} and R^{f} are each independently H or C1-6 alkyl;
Z¹, Z² and Z³ are each independently C or N;
R^{1e}-R^{5e}, R^{8e} and R^{9e} are unsubstituted or substituted with one or more substituents, in one embodiment 1-4 substituents, in another embodiment 1 or 2 substituents, selected from Q¹.

In another embodiment, the conjugates have formula: 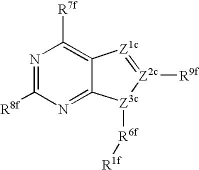
or a pharmaceutically acceptable derivative thereof, wherein, R^{6f} is C1-10 alkyl and optionally containing a heteroatom, C2-10 alkenyl or C2-10 alkynyl;
R^{1f} is Y or hydroxy;
R^{7f} is Y, H, hydroxy, halo, azido, C1-6 alkyl and optionally containing a heteroatom, C2-6 alkenyl, C2-6 alkynyl, SR^{d} or NR^{a}R^{c};
R^{8f} is Y, H, alkyl, SR^{d}, halo or NR^{a}R^{b};
R^{9e} is Y, H, or C1-6 alkyl;
Y is a linker-drug construct L-D, wherein L, which may or may not be present, is a non-releasing linker and D is a drug moiety;
R^{7f}, R^{8f} and R^{9f} are selected such that at least one of R^{1f}, R^{7f}, R^{8f} and R^{9f} is Y and at least one of R^{7f} and R^{1f} is OH;
Z^{1f}, Z^{2f} and Z^{3f} are each independently C or N;
R^{7f}, R^{8f} and R^{9f} are unsubstituted or substituted with one or more substituents, in one embodiment 1-4 substituents, in another embodiment 1 or 2 substituents, selected from Q¹.

In certain embodiments, the conjugates provided herein have formula (3): 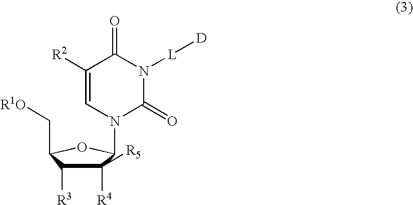
or a pharmaceutically acceptable derivative thereof, wherein R³, R⁴ and R⁵ are each independently H, optionally substituted hydroxy, halo, azido, C1-6 optionally substituted alkyl and optionally containing a heteroatom, C2-6 alkenyl or alkynyl;
wherein R² is H, C1-6 optionally substituted alkyl and optionally containing a heteroatom, C2-6 alkenyl or alkynyl, C3-6 cycloalkyl, optionally substituted hydroxy, aryl or heteroaryl;
formula (4): 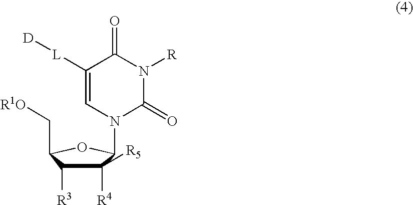
wherein R³, R⁴ and R⁵ are independently H, optionally substituted hydroxy, halo, azido, C1-6 optionally substituted alkyl and optionally containing a heteroatom, C2-6 alkenyl or alkynyl;
wherein R is H or C1-6 alkyl;
formula (5): 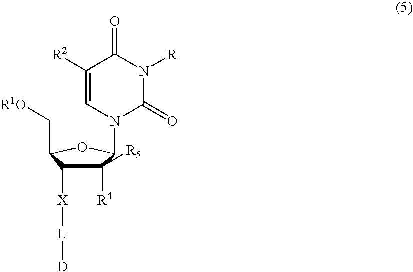
wherein R⁴ is H, optionally substituted hydroxy, halo, azido, C1-6 optionally substituted alkyl and optionally containing a heteroatom, C2-6 alkenyl or alkynyl; wherein R² is H, C1-6 optionally substituted alkyl and optionally containing a heteroatom, C2-6 alkenyl or alkynyl, C3-6 cycloalkyl, optionally substituted hydroxy, aryl or heteroaryl;
wherein R is H or C1-6 alkyl;
wherein X is O or is absent;
formula (6): 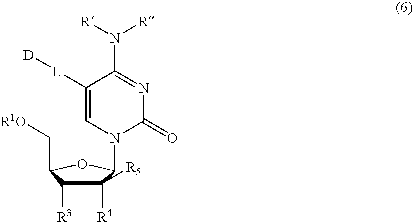
wherein R³, R⁴ and R⁵ are independently H, optionally substituted hydroxy, halo, azido, C1-6 optionally substituted alkyl and optionally containing a heteroatom, C2-6 alkenyl or alkynyl;
wherein each R′ and R″ are independently H or lower alkyl; formula (7): 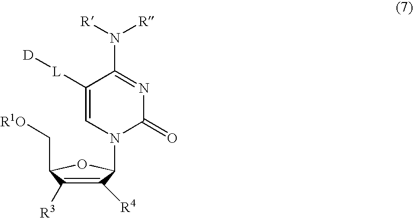
wherein R³ and R⁴ are independently H, optionally substituted, C1-6 optionally substituted alkyl and optionally containing a heteroatom, C2-6 alkenyl or alkynyl;
wherein R′ and R″ are independently H or lower alkyl;
   formula (8): 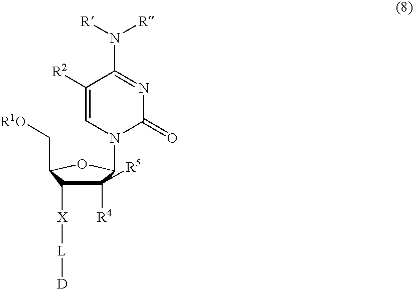
wherein R⁴ and R⁵ are independently H, optionally substituted hydroxy, halo, azido, C1-6 optionally substituted alkyl and optionally containing a heteroatom, C2-6 alkenyl or alkynyl;
wherein R² is H, C1-6 optionally substituted alkyl and optionally containing a heteroatom, C2-6 alkenyl or alkynyl, C3-6 cycloalkyl, optionally substituted hydroxy, aryl or heteroaryl;
wherein R′ and R″ are independently H or lower alkyl;
wherein X is O or is absent;
formula (9): 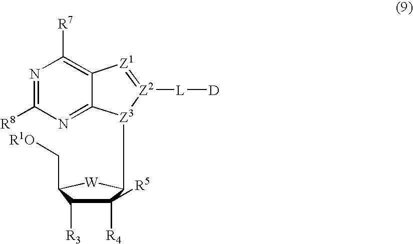
wherein R³, R⁴ and R⁵ are independently H, optionally substituted hydroxy, halo, azido, C1-6 optionally substituted alkyl and optionally containing a heteroatom, C2-6 alkenyl or alkynyl;
wherein R⁷ is H, optionally substituted hydroxy, halo, azido, C1-6 optionally substituted alkyl and optionally containing a heteroatom, C2-6 alkenyl, C2-6 alkynyl, NR′R′″, wherein R′ and R″ are independently H or lower alkyl, or SR″″, wherein R′″ is H or lower alkyl;
wherein R⁸ is H, halo, NR′R″, wherein R′ and R″ are independently H or lower alkyl, or SR″″, wherein R′″ is H or lower alkyl;
wherein W is C or O;
wherein Z¹, Z² and Z³ are independently C or N;
formula (10) 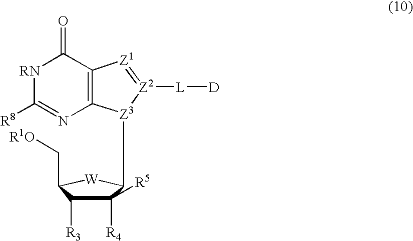
   wherein R³, R⁴ and R⁵ are independently H, optionally substituted hydroxy, halo, azido, C1-6 optionally substituted alkyl and optionally containing a heteroatom, C2-6 alkenyl or alkynyl;
wherein R⁸ is H, halo, NR′R″, wherein R′ and R″ are independently H or lower alkyl, or SR″″, wherein R′″ is H or lower alkyl;
wherein R is H or lower alkyl;
wherein W is C or O;
wherein Z¹, Z² and Z³ are independently C or N;
   or formula (11): 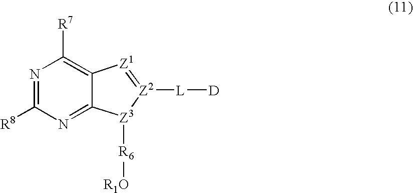
or a pharmaceutically acceptable derivative thereof, wherein R⁶ is an acyclic C1-10 alkyl optionally substituted alkyl and optionally containing a heteroatom, or C2-10 alkenyl or alkynyl;
wherein R⁷ is H, optionally substituted hydroxy, halo, azido, C1-6 optionally substituted alkyl and optionally containing a heteroatom, C3-6 cycloalkyl, C2-6 alkenyl, C2-6 alkynyl, NR′R′″, wherein R′ and R″ are independently H or lower alkyl, or SR″″, wherein R′″ is H or lower alkyl;
wherein R⁸ is H, halo or NR′R″, wherein R′ and R″ are independently H or lower alkyl; wherein W is C or O;
wherein Z¹, Z² and Z³ are independently C or N;
wherein for formulas 3-11 each L, which may or may not be present, is a non-releasing linker moiety;
each D is a drug moiety; and
each R¹ is H, or acyl.

In the above formulas 3-11, the linker-drug moiety may be attached at other positions of the pyrimidine or purine nucleoside analog.

In certain embodiments, the conjugates are paclitaxel-thymidine conjugates. In other embodiments, the paclitaxel-thymidine conjugates contain a non-releasing linker between paclitaxel and thymidine. In certain embodiments, the linker contains an alkylene chain or PEG chain. In certain embodiments, the linker is bonded to thymidine via a covalent bond. In certain embodiments, the linker is bonded to paclitaxel via a first functionality. In certain embodiments, the first functionality is a carbamate. In other embodiments, the substrate is conjugated to paclitaxel at C7 position. In one embodiment, the paclitaxel-thymidine conjugates have formula: 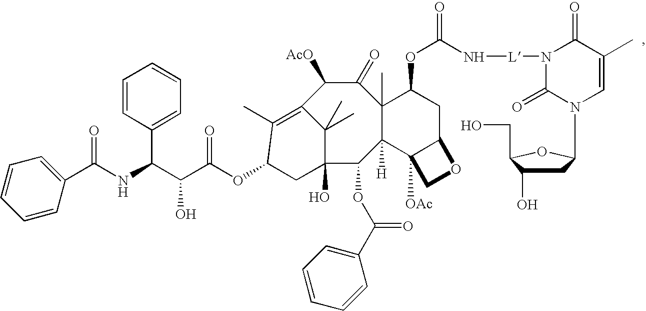
or a pharmaceutically acceptable derivative thereof.

In other embodiments, the conjugates are paclitaxel-thymidine conjugates in which the linker contains an alkylene chain or PEG chain and is bonded to thymidine via a covalent bond and to paclitaxel via a carbamate group at C10. In one embodiment, the paclitaxel-thymidine conjugates have formula: 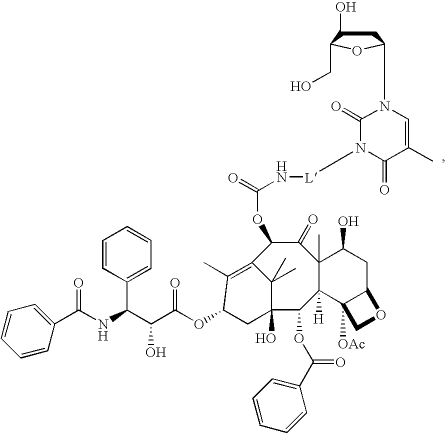
or a pharmaceutically acceptable derivative thereof.

In other embodiment, the paclitaxel-thymidine conjugates have formula: 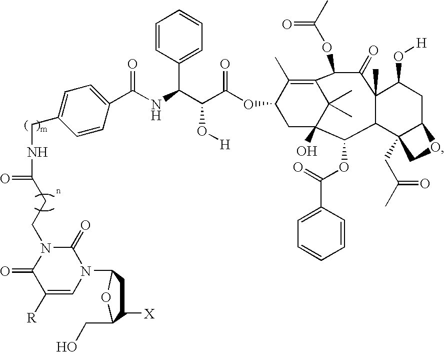
or a pharmaceutically acceptable derivative thereof, where n is 1, 2, 3, 4, 5, 6, 7, 8, 9, 10 or more.

In certain embodiments, the conjugates are vinblastine-thymidine conjugates. In certain embodiments, the conjugates are desacetyl vinblastine-thymidine conjugates. In certain embodiments, the vinblastine-thymidine conjugates contain a non-releasing linker between vinblastine and thymidine. In certain embodiments, the linker contains an alkylene chain or PEG chain. In certain embodiments, the linker is bonded to thymidine via a covalent bond. In other embodiments, the linker is bonded to C3 of vinblastine via an amide group. In one embodiment, the vinblastine-thymidine conjugates have formula: 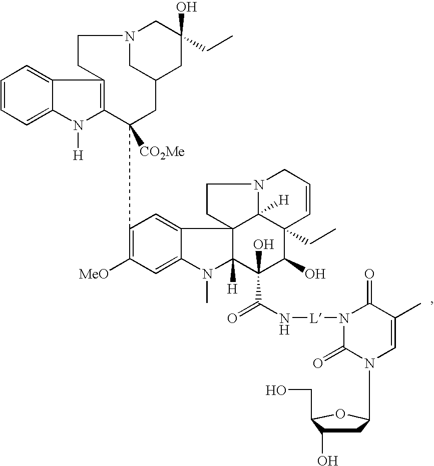
or a pharmaceutically acceptable derivative thereof,

In certain embodiments, the conjugates are doxorubicin-thymidine conjugates. In certain embodiments, the doxorubicin-thymidine conjugates contain a non-releasing linker between doxorubicin and thymidine. In certain embodiments, the linker contains one or more groups containing maleimide, an alkylene chain and PEG chain. In certain embodiments, the linker is bonded to thymidine via a covalent bond. In other embodiments, the linker is bonded to doxorubicin via an amino group. In one embodiment, the doxorubicin-thymidine conjugates have formula: 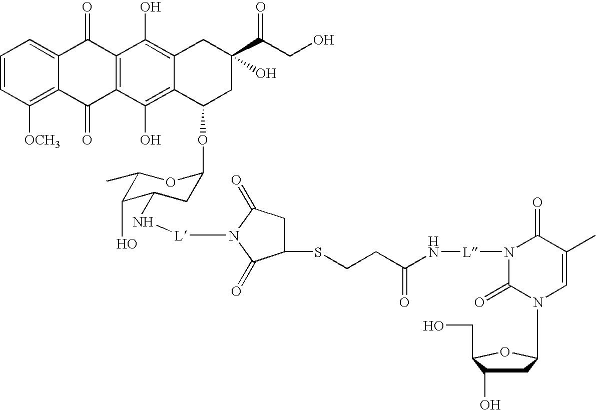
where L′ and L″ are each independently selected from alkylene or PEG or a pharmaceutically acceptable derivative thereof.

More examples of conjugates provided herein are provided in Table ?

C. Preparation of the Conjugates

The conjugates provided herein can be prepared using any convenient methodology. In one approach, the conjugates are produced using a rational approach. In a rational approach, the conjugates are constructed from their individual components (e.g., drug, linker precursor and substrate). The components can be covalently bonded to one another through functional groups known in the art. Furthermore, the particular portion of the different components modified to provide for covalent linkage will be chosen so as not to substantially adversely interfere with that component's desired binding activity. For example, in a drug moiety, a region that does not affect the target binding activity will be modified, such that a sufficient amount of the desired drug activity is preserved.

The functional groups can be present on the components or introduced onto the components using one or more steps, such as oxidation, reduction, cleavage reactions and the like. Examples of functional groups that can be used in covalently bonding the components to produce the conjugate include but are not limited to hydroxy, sulfhydryl, amino, carbonyl, and the like. Where desirable, certain moieties on the components may be protected using blocking groups, as is known in the art, see, e.g., Green & Wuts, Protective Groups in Organic Synthesis (John Wiley & Sons) (1991).

In the following discussion nucleoside and nucleoside analog are interchangeable terms. For the purpose of teaching the use of the conjugates, Scheme 1 illustrates the general syntheses of linker nucleoside constructs with linker attachment at N-3 of a pyrimidine base of a nucleoside, followed by attachment of a drug to the linker-nucleoside construct.

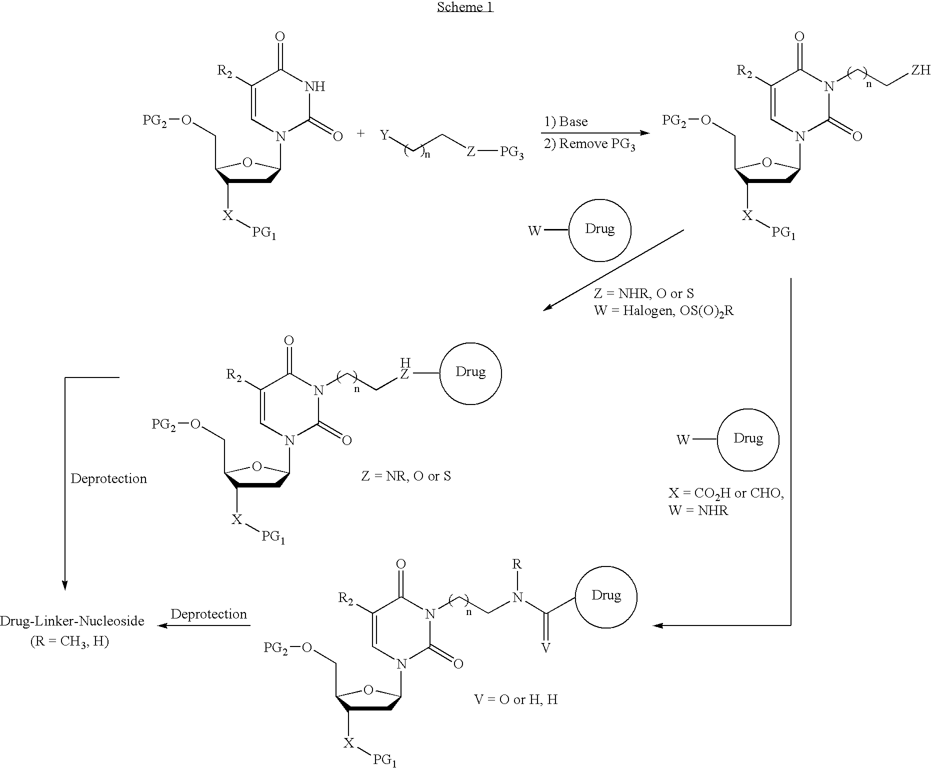

Examples of functional groups on the drug for attaching to the linker-nucleoside construct include, but are not limited to, COOH, CHO, halogen, NHR, or OH, wherein m is a positive integer preferably between 1 and 20. The drug, linker precuser, and nucleoside used in the illustrated methods of conjugation are suitably protected in a manner consistent with the conditions required to affect conjugation, and the appropriate choice of protecting groups are within the ordinary skill of one in the art of chemical synthesis.

Furthermore, the methylenes interposed between the two ends of the linker are for illustrative purposes only, and should not be construed as a limitation to the conjugates provided herein. For example the methylene subunits may be switched to ethyleneoxy subunits to give a polyethylene glycol based linker. The linker nucleoside construct is prepared by the condensation of thymidine or a suitable nucleoside analog with a linker precusor having a first end and a second end wherein the first end contains an appropriate leaving group and the second end contains a suitably protected functional group. Condensation is effected with a base with either the hydroxyl groups of the nucleoside carbohydrate protected or in free form. The intermediate with carbohydrate hydroxyl groups suitably protected is then subjected to deprotection to selectively remove a protecting group on the functional group on the second end of the linker.

With COOH as the functional group on the drug, and an amine as the free functional group on the linker precursor, attachment of the linker-nucleoside construct to the drug uses amide bond coupling procedures well known in the art of peptide chemistry. Where the functional group on the drug is CHO, reductive amination is performed with NaBH₄, NaCNBH₄ NaB(OAc)₃H or other suitable reducing groups to provide the drug conjugate. Where the functional group on the drug is OH, coupling is affected by activation of the linker COOH group with DCC, or with any other acid activation agent well known in the art for ester bond formation. Where a functional group on the drug is halogen, alkylsulfonyloxy, arylsulfonyloxy, or any other suitable leaving group for nucleophilic displacement, conjugation is through nucleophilic displacement by the free amine of the linker-nucleoside construct in the presence of Et₃N or any other appropriate acid scavenger.

When the free functional group on the linker of the linker-nucleoside construct is a thiol, condensation with the drug is effected by nucleophilic displacement of a leaving group on the drug, as given for the aforementioned nucleophilic displacement by free amine, after conversion of the thiol to a thiolate. The procedure for drug conjugation to a linker-nucleoside construct bearing a free thiol groups is also applicable to a free hydroxyl group on the linker to give a drug-linker-nucleoside conjugate with ether attachment of the linker to the drug.

The conjugates may also be constructed using the same aforementioned chemical transformations for synthesis of drug-linker-nucleophile conjugates by first attaching the linker to the drug followed by attachment to N-3 of the nucleoside base. The purpose of the linker is to serve as a spacer between the drug and the nucleoside in order to remove or relieve steric interactions that may interfere with the kinase substrate activity of the nucleoside and/or the pharmacological effect of the drug so effective amounts of kinase and pharmacological activities remain. The linker may also be chosen based on its effect on the hydrophobicity of the drug conjugate to improve passive diffusion into the target cells or tissue. It should also be understood that cleavage of a bond within a linker to generate free drug is not required for the pharmacological effect of the drug that is incorporated into a conjugate.

Example syntheses of nucleoside-linker constructs with linker attachment to the nucleoside C3′-O is generally illustrated in Scheme 2a.

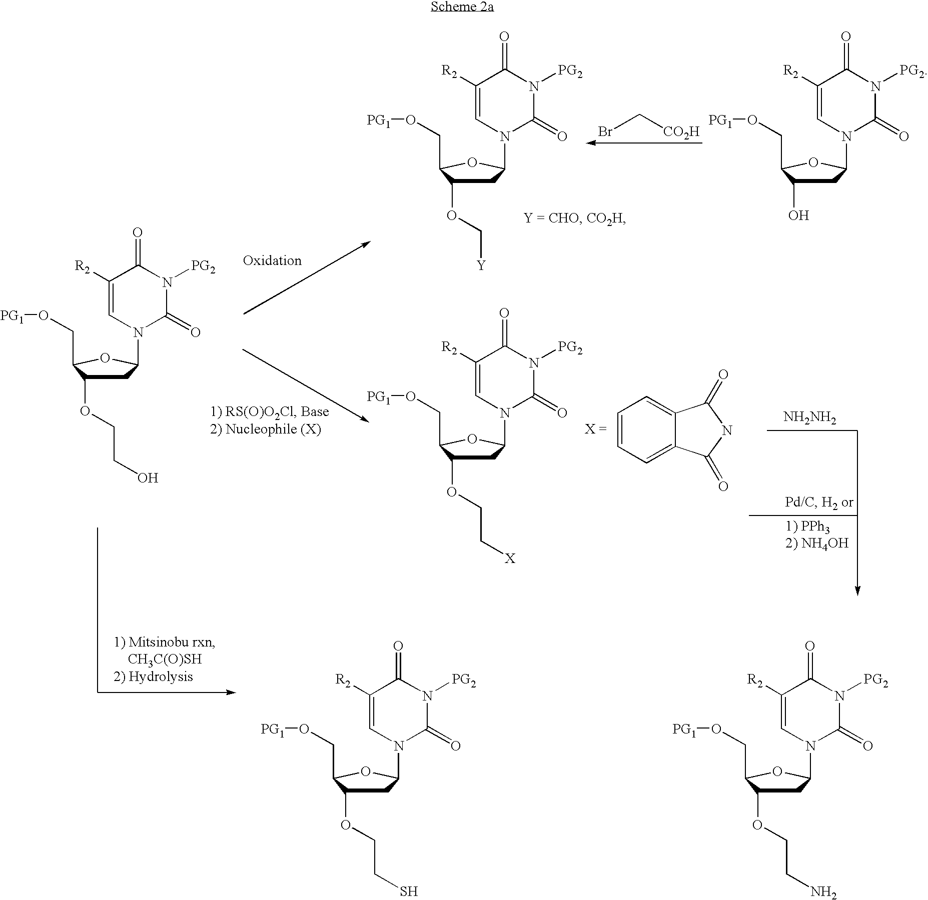

In one example, nucleoside constructs may be synthesized from known 3′-O-(2-hydroxyethyl)-2′-deoxy-thymidine suitably protected at C5′-O and N3. In another example, nucleoside constructs may be synthesized from a suitably protected C5′-O, N3 uridine or a suitably protected C5′-O, C4-N cytidine wherein the hydroxyethyl group is introduced as reported for 2′-deoxy-thymidine. The hydroxyethyl group is transformed by Swern oxidation to provide an aldehyde functional group. A carboxylic acid functional group may be introduced by reaction of a suitably protected 2′-deoxy-ribonucleoside with ClCH₂COONa (Edge, M. D., et al. J. Chem. Soc. Perkin Trans. 1, 290-4 (1973)).

The aldehyde or carboxylic acid functional groups may be further elaborated to extend the linker, or may be used directly for attachment to a drug bearing an amino group. Additionally, the hydroxyethyl group may be activated for nucleophilic displacement, for example, by treatment with triflic anhydride. Nucleophilic displacement may be from a drug nucleophile to give a drug-linker-nucleoside construct, or with the anion derived from a protected amine including phthalimido or bis-(t-butyloxycarbonyl)amine, to introduce a protected amine. After selective removal of the amine protecting group or groups, the free amine may be used to further extend the linker or may be used to attach the linker to the drug as described for Scheme 1. An alternative method to introduce the amino functional group and a method to introduce a thiol functional group are given in Teng, K., et al. U.S. Pat. No. 6,087,482 the disclosure of which is incorporated by reference.

Alternatively, drug-linker nucleoside constructs with linker attachments at C3′-O may be synthesized by selectively condensing thymidine or 2′-deoxyuridine with suitable protection at C5′-OH, or 2′-deoxy-cytosine with suitable protection at C5′-OH and C4-NH₂, using a linker precursor bearing an electrophile on a first end and a suitably protected functional group on a second end. In one example, the electrophile is a chloromethyloxy group (Scheme 2b). 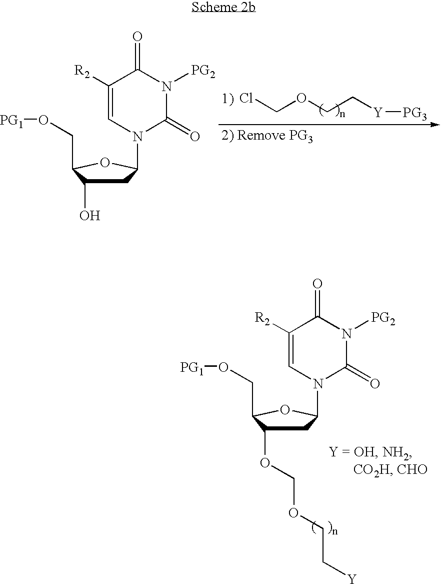
where n is 1-30.

Selective deprotection of the functional group on the linker in the linker-nucleoside conjugate would provide a functional group for attachment to a drug as described for Scheme 1. Alternatively the second end is a functional group such as a selectively protected OH, N3, ester or a terminal alkene which may be converted once incorporated into the nucleoside-linker construct into an electrophile by for example selective deprotection to give a free OH which is transformed to an aldehyde by Swern oxidation or is sulfonated to provide a leaving group, Staudinger reduction of the azide to give an amine, selective hydrolysis of the ester to give a carboxylic acid, or ozonolysis of the terminal alkene to provide an aldehyde or hydroboration to give a terminal OH which is further manipulated as described.

Example syntheses of nucleoside-linker constructs with direct linker attachment to the nucleoside C3′ carbon is generally illustrated in Scheme 2c.

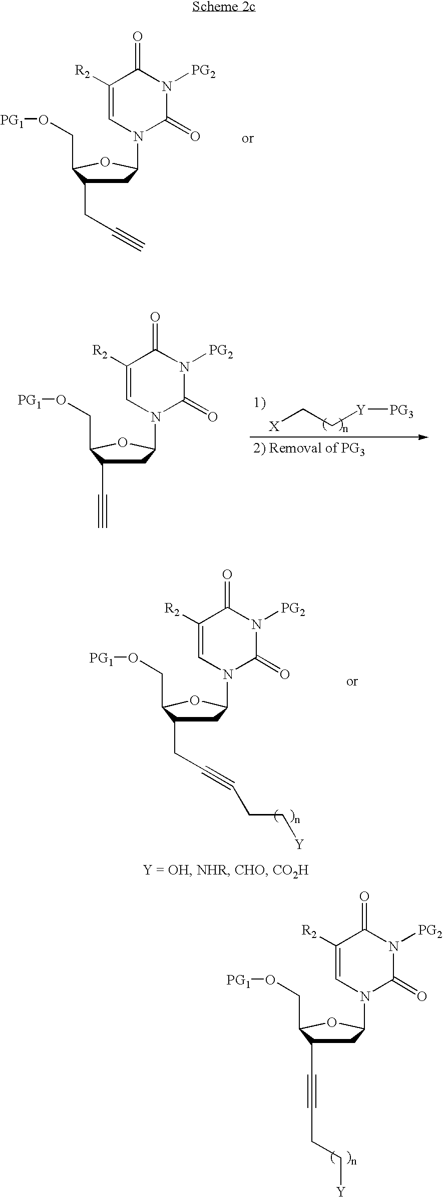

The known C3′-alkynyl or C3-methyl alkynyl thymidine with suitable protection is treated with an appropriate base such as KH to generate the acetylide which is condensed with a molecule with a third and a four end wherein the third end is an electrophile such as an arene- or alkane sulfonate or a halogen and the fourth end is an appropriately protected functional group thereby extending the linker attached to C3′. Selective deprotection would then give a functional group of the nucleoside-linker for further extension of the linker or to attach the linker to the drug as described for Scheme 1. Prior to selective deprotection of the functional group, or after formation of the drug conjugate the alkyne may be reduced to the alkene or alkane to proved further drug conjugates. Alternative methods to synthesize nucleosides having a C3′-C bond wherein the substituent to C3′ contains a suitable functional group for drug attachment as described for Scheme 1 are given in Teng, K., et al. U.S. Pat. No. 6,087,482 the disclosure of which is incorporated by reference.

For conjugates with linker attachment at C-5 Scheme 3 teaches the synthesis of linker-nucleoside constructs at C-5. In one route given in general in Scheme 3a the known 5-iodouracil with protected C3′ and C5′ hydroxyl groups or the hydroxyl groups in free form is condensed with a alkyne on the first end of the linker through a Sinorogoshi coupling using an appropriate palladium catalyst well know in the art of Pd catalyzed cross coupling reactions to give a linker-nucleoside conjugate with direct attachment of a linker to a nucleoside through a carbon-carbon bond.

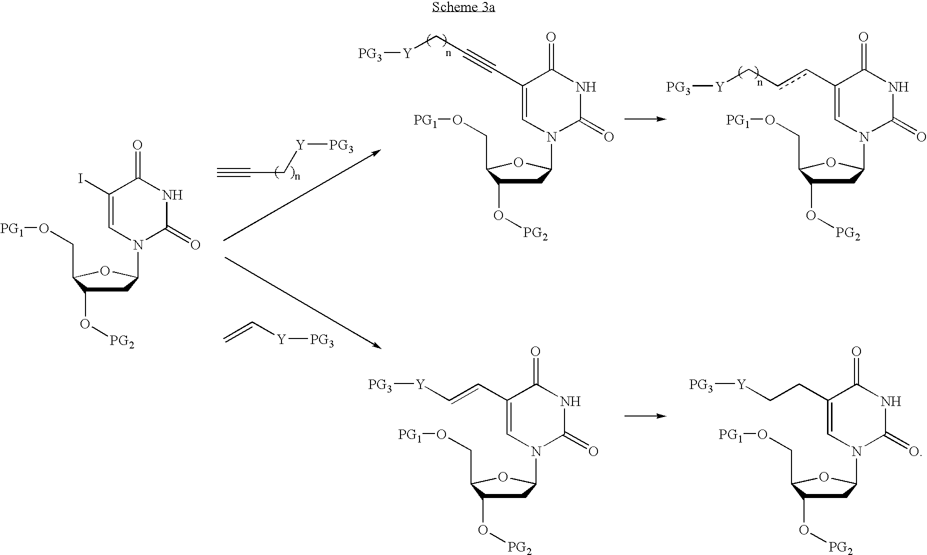

An alternative route for linker attachment to C5 through a carbon-carbon bond employs the Heck reaction using an alkene on the first end of the linker. An example of an alkene based linker with an appropriate second end is ethyl acrylate. In this example hydrolysis with optional reduction of the double bond would provide a COOH group for further linker elaboration or attachment to a drug a described for Scheme 1 to give a drug-linker-nucleoside conjugate either with alkene or alkane functionality within the linker. Reduction of the alkyne in either the aforementioned linker-nucleoside construct or in a final drug conjugate would also provide linkers having alkene and alkane functionality within the linker. Conjugates with linker attachment at C-5 of 2′deoxy-cytidine are made in similar fashion starting from 5-iodo-2′deoxycytidine.

An alternative to nucleoside drug conjugates with linker attachment to C5 is shown in general in Scheme 3b and begins with 5-thio-2-deoxy uracil.

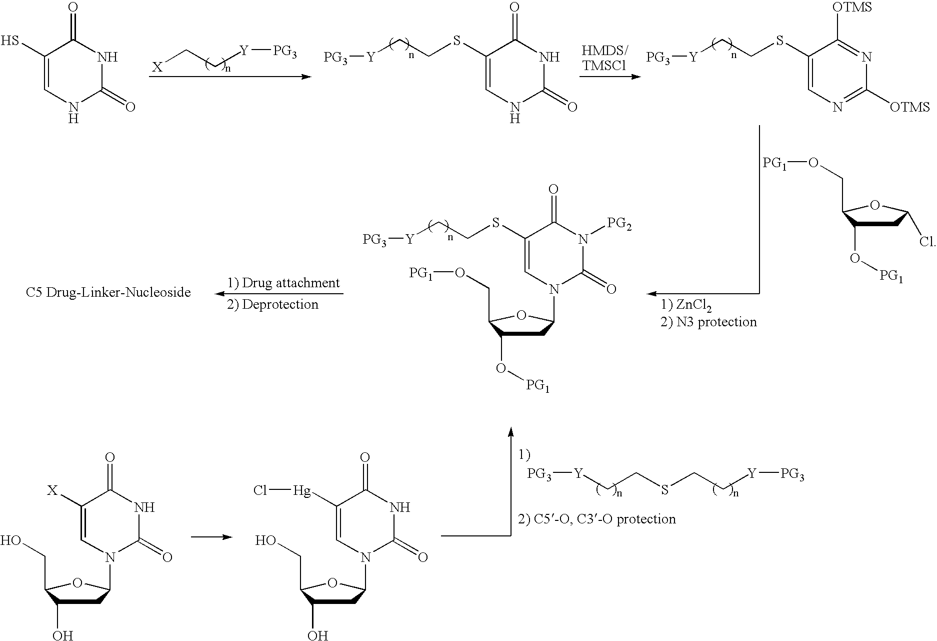

Alkylation with a bi-functional molecule having an electrophile at a third end such as halogen, alkyl or arenesulfonylchloride or a Michael acceptor and a protected nucleophile at a fourth end provides a S-alkylated pyrimidine base wherein a linker is attached to C5. The nucleoside linker construct is then formed by silylation of the pyrimidine base with hexamethyldisilazane and a catalytic amount of trimethylsilylchloride followed by condensation with an appropriately protected α-D-ribosylchloride using ZnCl2 in CCl₄. Alternatively, 5-thio-2′deoxy-urudine may be S-alkylated to directly give the nucleoside-linker construct. For the bifunctional molecule the fourth end may be a functionality that may be converted to a electrophile or nucleophile once incorporated into a nucleoside-linker construct and includes but is not limited to a selectively protected OH, N3, ester or a terminal alkene. Selective deprotection or conversion of the functional group in the linker of the nucleoside-linker construct as described would provide a functional group for attachment to a drug as described for Scheme 1. An alternative route to the general structure in Scheme 3b is through halogen-metal exchange of a 5-halo-2′deoxyuridine suitably protected or in free form followed by cross-coupling with a symmetrical di-sulfide bearing a suitably protected functional group (see Bashkin, J. K., et al. J. Org. Chem. 55: 5125-5132 (1990) and Bergstom, D. J. Amer. Chem. Soc. 111: 374-375 (1989)). Selective deprotection of the functional group incorporated in the nucleoside-liker construct provides a functional group which may be used to further extend the linker or may be used to attach the linker to the drug as described for Scheme 1.

It should be appreciated that thymidine or uracil nucleoside constructs or drug conjugates so described may be converted to their corresponding 2′deoxy cytidine analogs by treatment with a chlorinating agent such as POCl3 to form a 4-Cl pyrimidine intermediate which is condensed with ammonia or a substituted amine. Syntheses of exemplary drug-linker-nucleoside conjugates for paclitaxel are given in Schemes 4 and 5 to further teach the conjugates provided herein. In Scheme 4a paclitaxel, appropriately protected at the more reactive C2′ hydroxyl, is condensed with a linker-nucleoside construct wherein n is 0 or a positive integer, preferably where n is between 0 and 20, wherein the second end of the linker bears a carboxylic acid group.

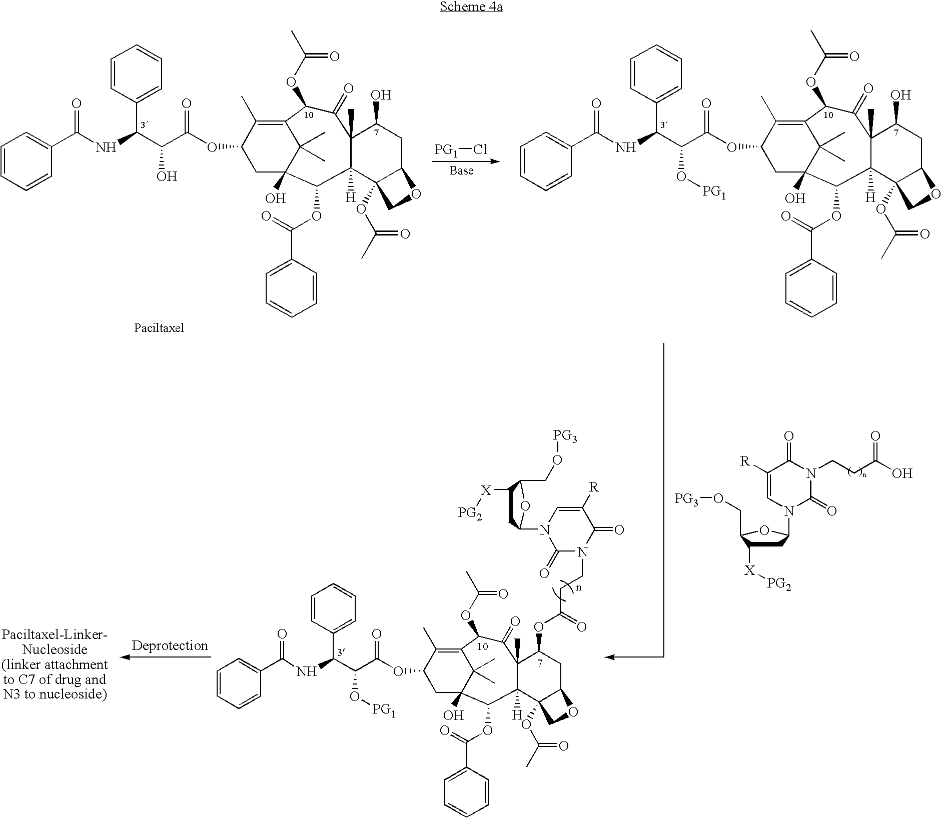

Condensation of the second end to the protected taxane is by DCC or any other appropriate coupling agent used for ester bond formation. Deprotection then gives the paclitaxel-linker-conjugate with linker attachment at C7 of paclitaxel.

In Scheme 4b, the known paclitaxel derivative having a free C10-OH and C2′OH and C7-OH groups protected as silyl ethers is condensed with the linker-nucleoside construct of Scheme 4a to form an ester bond at C10. 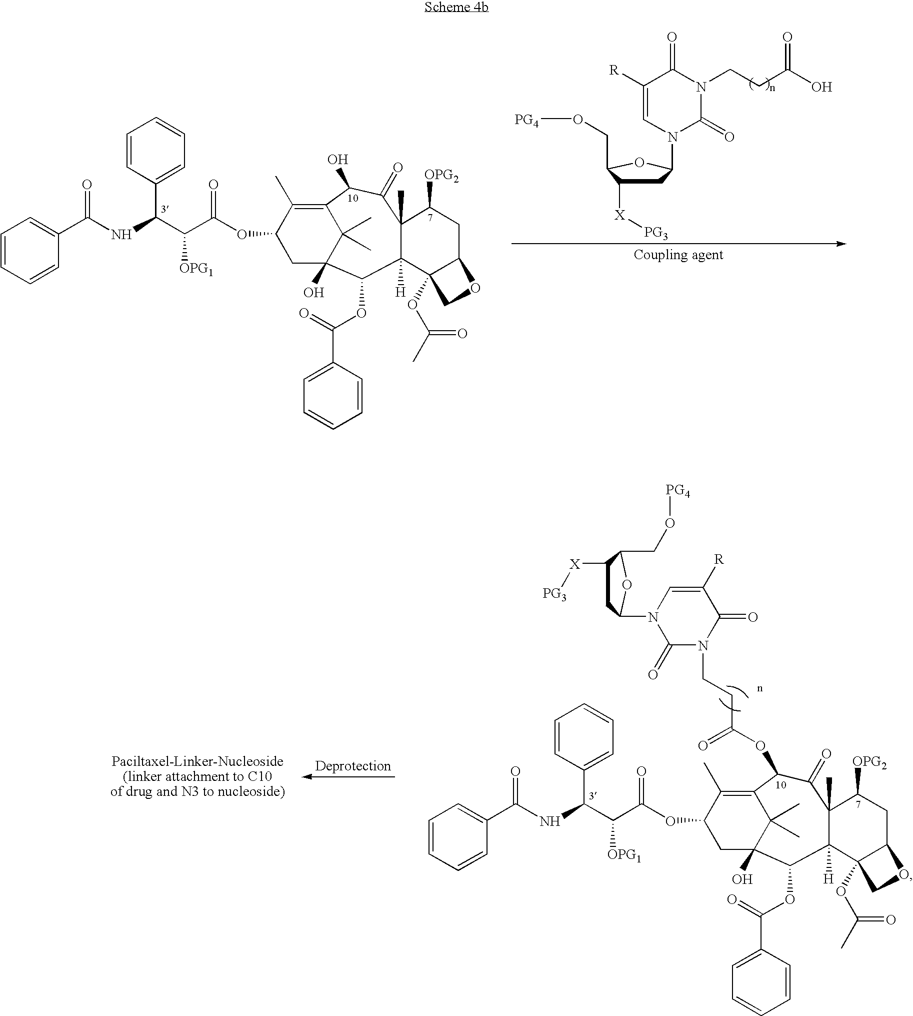
where n is 0-30.

Following the general procedures as described for Scheme 4a the paclitaxel-linker-nucleoside conjugate with linker attachment at C-10 is obtained.

In Scheme 5a, Baccatin III, appropriately protected at C7 is condensed with an appropriately protected phenylisoserine using standard ester bond forming conditions to give an intermediate that is deprotected to give the free C3′ amino group.

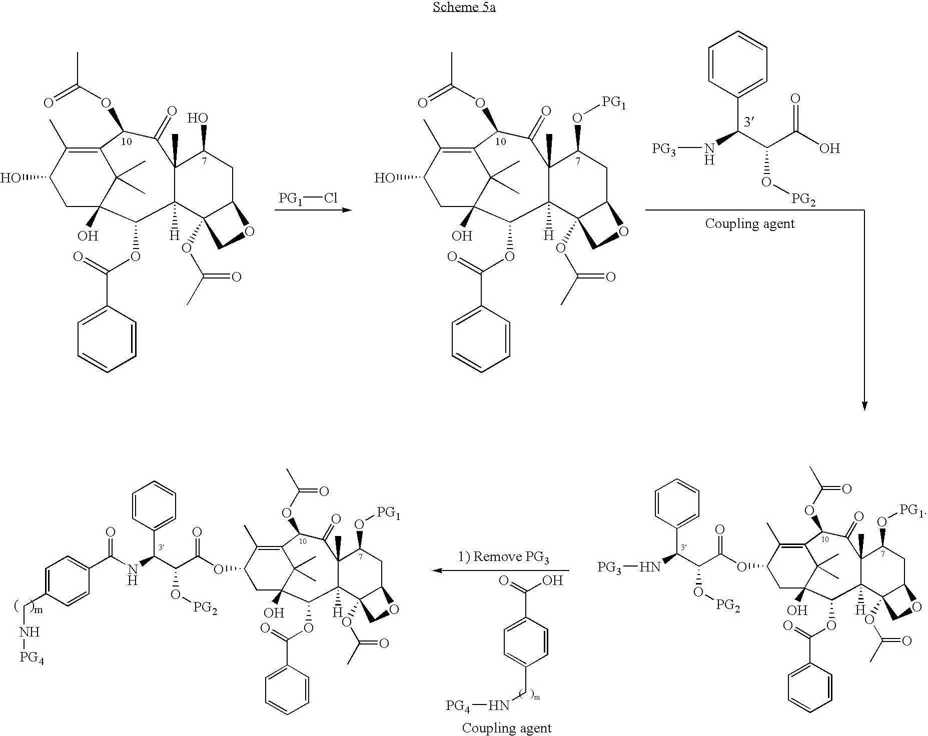

Condensation with a benzoic acid derivative containing a suitably protected amine wherein m is preferentially zero, one or two using standard amide bond forming conditions then provides a paclitaxel derivative wherein a functionality amenable to conjugation is introduced into the C3′-N benzamido group. Deprotection of the amine followed by amide bond formation to a linker-nucleoside conjugate wherein the second end of the linker bears a COOH group gives after final deprotection the desired paclitaxel-linker-nucleoside conjugate with attachment at the C3′-N benzamido group.

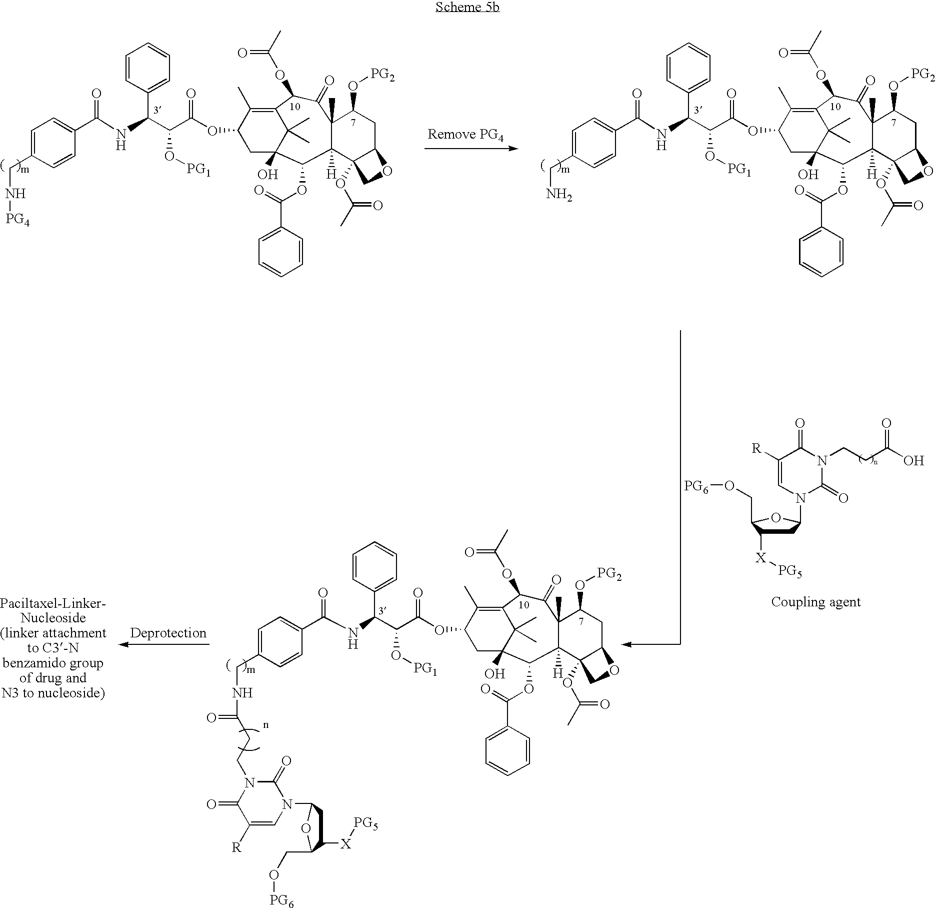

Drug-linker-purine nucleoside conjugates corresponding to Formulas 9, 10 and 11 are prepared according to the procedures illustrated in Schemes 1, 4 and 5 using a purine nucleoside linker construct wherein the linker is attached to C8 of the purine. Appropriate starting materials are exemplified by known compounds 8-bromo-2′deoxy-adenosine and 8-bromo-2′-deoxy-guanosine. For the syntheses of purine nucleoside-linker constructs according to Scheme 1, Y is a nucleophile, preferably a thiolate, which displaces the C8-Br and becomes incorporated into the purine nucleoside-linker construct, and Z is an appropriately protected nucleophile or electrophile which may be used in the reaction sequences exemplified by Schemes 4 and 5 after selective deprotection.

Method for Preparation of Paclitxel C10 Carbamates

Existing examples of paclitaxel C-10 carbamates prepared directly from paclitaxel include some simple analogs derived from 10-O-deacetyl-7-0, 10-O-bis-[N-(2,2,2-trichloroethyloxy)-aminocarbonyl]-paclitaxel as reported in Bourzat, J. Det al.; EPO Application 524,093 (1993). This synthetic methodology, however, is not versatile since selective reaction of the amine input at C-10 is possible only in dichloromethane. A more general approach for the synthesis of C-10 carbamates starts from 10-deacetyl-baccatin-III. However, subsequent steps to install the phenylisoserine side chain are problematic for amine inputs containing additional functional groups that require protection. Due to the chemical sensitivity of the taxane core, the protecting group strategy required for such amine inputs would be complex. Disclosed in the instant application is a method which permits the use of amine inputs containing additional functionality in free form. The disclosed method allows for the syntheses of C10 carbamates directly from paclitaxel that otherwise would be inaccessible or difficult to prepare.

A procedure for preparation of Paclitaxel C10 carbamates as provided herein is illustrated in Schemes 7 and 8. Accordingly, compound 5a can be converted in nearly quantitative yield into its C₁₀ carbonylimidazole 6a by reaction with carbonyldiimidazole (CDI) in dichloromethane at room temperature. Compound 6a can be reacted with amines in suitable solvents to yield the corresponding carbamate 8a, which can be deprotected to give 9a. Typically, for primary and secondary amines, the reaction can be carried out in non-polar solvents, such as dichloromethane or in protic solvents such as IPA or t-BuOH at elevated temperatures. 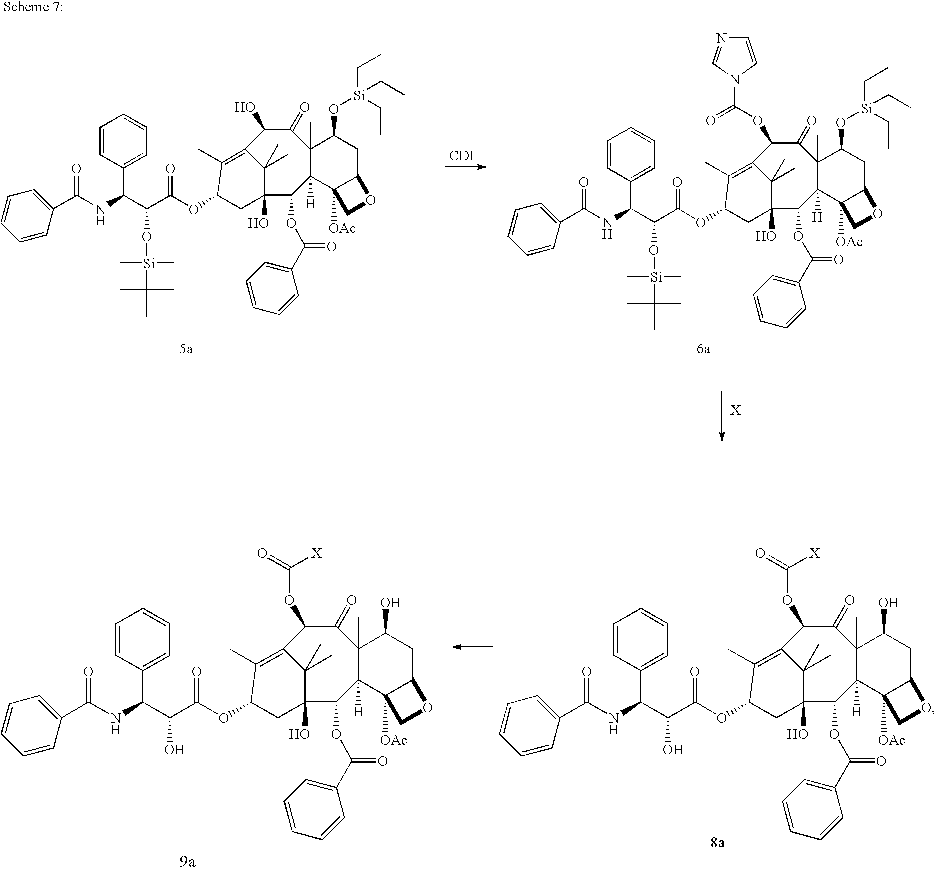
where X is an amine.

In certain embodiments, the C10-carbonylimidazole 6a can be activated with an alkylating agent such as an alkyl halide, alkyl sulfonate or di-alkyl-sulfate to give a N¹-alkyl-N³-acyl imidazolium species represented by 7a of Scheme 8. In certain embodiments the alkylating agent is selected from dimetylsulfate and methyl iodide. The imidazolium species can then be reacted with various amines either in free or salt forms in protic solvents or aprotic solvents such as DMF, DMSO or dioxane. For amine salts condensation with 7a is conducted in the presence of a hindered base such as DIEA. In certain embodiments, less reactive amines, such as arylamines or heteroarylamines may be condensed with 7a to obtain paclitaxel C10 carbamates with N-aryl or N-heteroaryl linker attachment.

Various nucleophiles can be used in the reactions provided herein to prepare C10 paclitaxel carbamates. Certain exemplary nucleophiles include, but are not limited to, primary and secondary amines, amine containing acids, such as α-amino acids, amino-sugars, such as glucosamine, arylamines, heteroarylamines, and α,α-disubstituted alcohols.

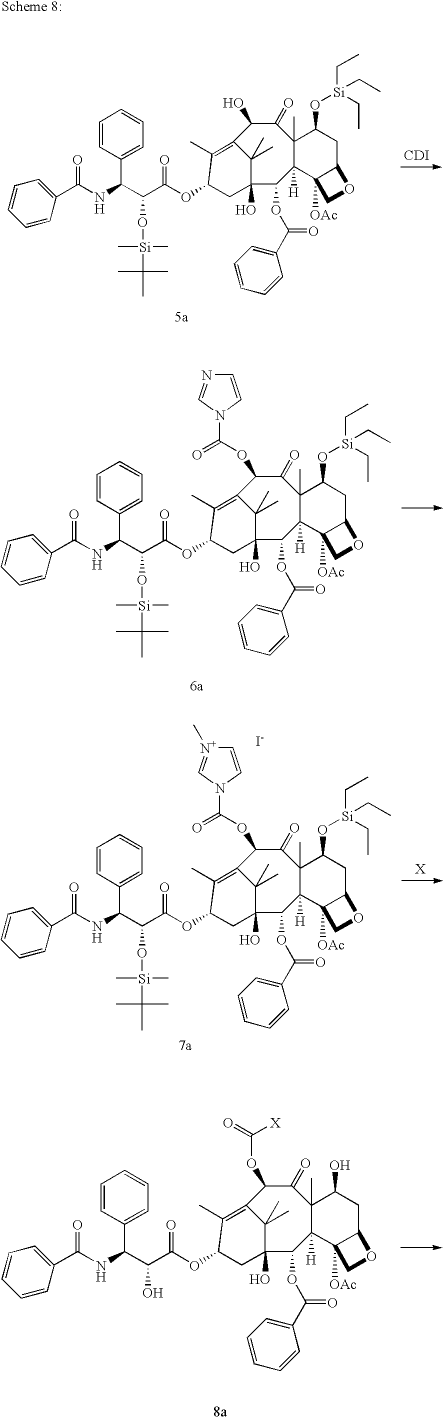

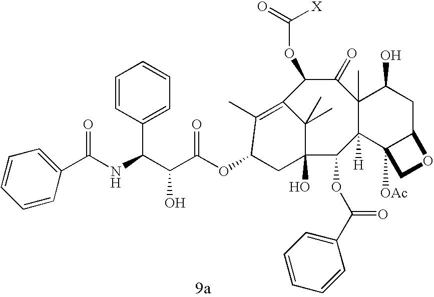

The following reaction schemes illustrate general methods for the preparation of conjugates provided herein.

a. Preparation of Thymidine-Linker Constructs 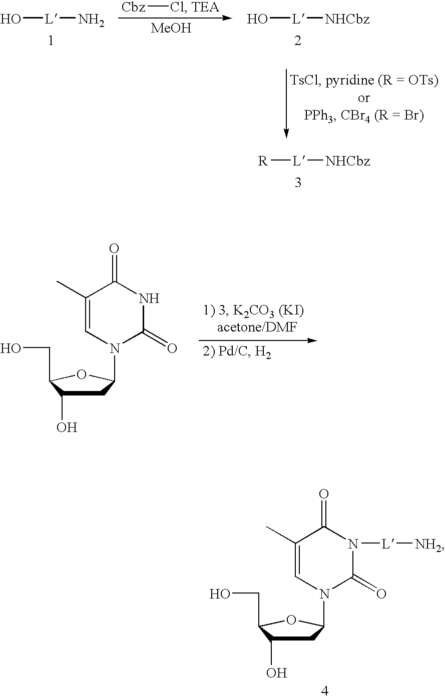
where L′ represents atoms between the first functionality and the second functionality of the linker moiety.

i) Reaction of Aminoalcohols with Benzylchloroformate

To an aminoalcohol (1, 100 mol %) in MeOH is added benzylchloroformate (150 mol %) and triethylamine (150 mol %). The reaction mixture is stirred for 16 h at RT then concentrated to dryness to give a residue which is purified by silica gel chromatography resulting in a mono-Cbz-protected aminoalcohol of structure 2.

ii) Reaction of N-Cbz-Protected Aminoalcohols with Tosylchloride or Triphenylphosphine and Carbon Tetrabromide

For R=OTs, to a mono Cbz-protected aminoalcohol (2, 100 mol %) in pyridine is added tosylchloride (100 mol %) at 0° C. The reaction mixture is stirred for 16 h while the solution is warmed up to RT then partitioned between ethyl acetate and water. The aqueous layer is extracted with ethyl acetate and the organic layer is dried over Na₂SO₄ and concentrated to dryness to give a residue which is purified by silica gel chromatography. Alternatively, for R=Br, to the mono Cbz-protected aminoalcohol (2, 100 mol %) in DCM are added triphenylphosphine (100 mol %) and carbon tetrabromide (100 mol %). The reaction mixture is stirred for 90 min at RT then concentrated to dryness to give a residue which is purified by silica gel chromatography resulting in a N-Cbz-protected amino linker of general structure 3.

iii) Reaction of Thymidine with N-Cbz-Protected Amino Bromides or Tosylates

To thymidine (100 mol %) in acetone/DMF are added a N-Cbz-protected amino linker precursor (3, 100 mol %) and K₂CO₃ (200 mol %) (for R=Br, KI [0.15 mol %] is also added). The reaction mixture is stirred at 50° C. for 48 h then partitioned between ethyl acetate and water. The aqueous layer is extracted with ethyl acetate and the organic layer is dried over Na₂SO₄ and concentrated to dryness to give a residue which is purified by silica gel chromatography. The thymidine-linker intermediate so obtained is subjected to catalytic hydrogenation using methanol with 10 wt % palladium on carbon and stirring under an atmosphere of H₂ for 16 h. Filtration of the reaction mixture on Celite, removal of volatiles in vacuo and lyophilization provides the thymidine-linker-amine intermediate of general structure 4.

b. Preparation of Paclitaxel-Linker-Thymidine Conjugates with Carbamate Linker Attachment at Paclitaxel C7

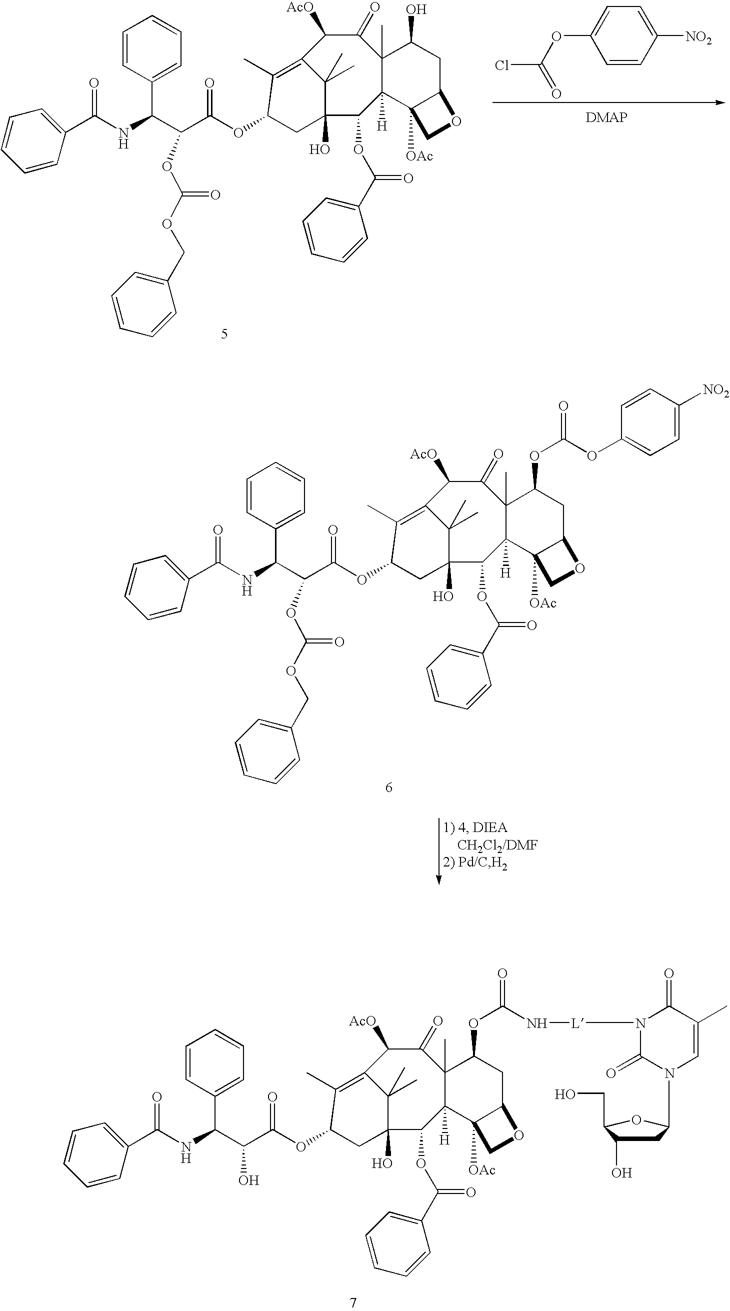

i) Preparation of 7-O-(p-nitrophenyloxycarbonyl)-paclitaxel (6)

To 2′-(benzyloxycarbonyl)-paclitaxel prepared according to the procedure described in. Chen, S.-H., et al., Tetrahedron (1993) 49: 2805-2828, dissolved in DCM are added p-nitrophenylchloroformate and DMAP. The reaction mixture is stirred for 1 h and concentrated to dryness. The resulting residue is purified by silica gel chromatography column eluting with 1:1 hexanes:ethyl acetate to give 6.

ii) Reaction of 7-O-β-nitrophenyloxycarbonyl)-paclitaxel with Thymidine-Linker-Amines

To 2′-O-(benzyloxycarbonyl)-O-7-(p-nitrophenyloxycarbonyl)-paclitaxel (6, 100 mol %) is added a thymidine-linker-amine (4, 100 mol %) dissolved in DMF followed by DIEA (1000 mol %) prepared as described above. The reaction mixture is stirred for 90 min then partitioned between ethyl acetate and water. The aqueous layer is extracted with ethyl acetate and the organic layer dried over Na₂SO₄ and concentrated to dryness to give a residue directly injected onto a preparative RP-HPLC C-18 reversed phase column for purification (Method A). Fractions containing the appropriate mass, as determined by analytical HPLC-MS (Method B), are pooled and the solvent is removed under reduced pressure. The 2′-O-(benzyloxycarbonyl)-paclitaxel(C7-carbamoyl)-linker-thymidine so obtained is subjected to catalytic hydrogenation using MeOH and HCl (200 mol %, introduced as a 1 M aqueous solution) with 10 wt % palladium on carbon and stirring under an atmosphere of H₂ for 16 h. Filtration of the reaction mixture on Celite, removal of volatiles in vacuo and lyophilization provides the paclitaxel-(C7-carbamoyl)-linker-thymidine conjugate of general structure 7.

c. Preparation of Deacetyl-Vinblastine-Linker-Thymidine Conjugates with Amide Linker Attachment at C3 of Vinblastine

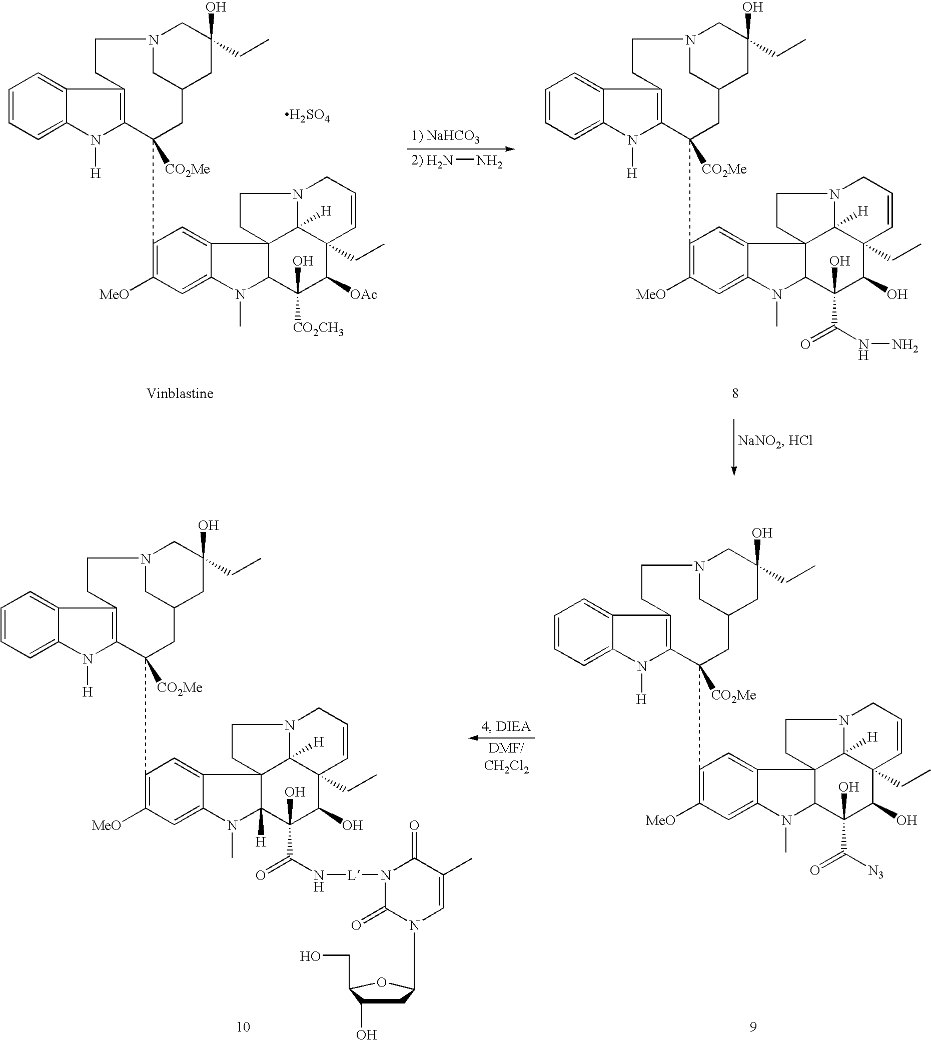

i) Synthesis of O⁴-3-de-(methoxycarbonyl)-vinblastin-3-yl-carbonyl Azide (9)

O⁴-3-de-(methoxycarbonyl)-vinblastin-3-yl-carbonyl hydrazide (8), prepared according to the procedure described in Bhushana, K. S. P Rao, et al., J. Med. Chem. (1985) 28: 1079, is dissolved in a mixture of methanol and an aqueous 1 M HCl solution. The solution is cooled to −10° C. and then NaNO₂ is added at once with stirring. After 10 min the pH of the brownish-red solution is adjusted to 8.8 with a saturated aqueous sodium bicarbonate solution and is extracted rapidly with DCM and washed with a saturated aqueous NaCl solution. The extracts are dried over Na₂SO₄ and concentrated. The solution of deacetylvinblastine acid azide (9) is used directly in the next step.

ii) Reaction of O⁴-3-de-(methoxycarbonyl)-vinblastin-3-yl-carbonyl Azide with Thymidine-Linker-Amines

To a DCM solution of deacetylvinblastine acid azide 9, (100 mol %) is added a DMF solution of a thymidine-linker-amine (4, 200 mol %) followed by DIEA (200 mol %). The reaction mixture is stirred for 3 h then partitioned between ethyl acetate and water. The aqueous layer is extracted with ethyl acetate and the organic layer is dried over Na₂SO₄ and concentrated to dryness to give a residue directly injected onto a preparative RP-HPLC C-18 reversed phase column for purification (Method A). Fractions containing the appropriate mass, as determined by analytical HPLC-MS (Method B), are pooled and CH₃CN is removed under reduced pressure. The remaining aqueous mixture is lyophilized to give vinblastine-linker-thymidine conjugate of general structure 10.

d. Preparation of Paclitaxel-Linker-Thymidine Conjugates with Carbamate Linker Attachment at Paclitaxel C10

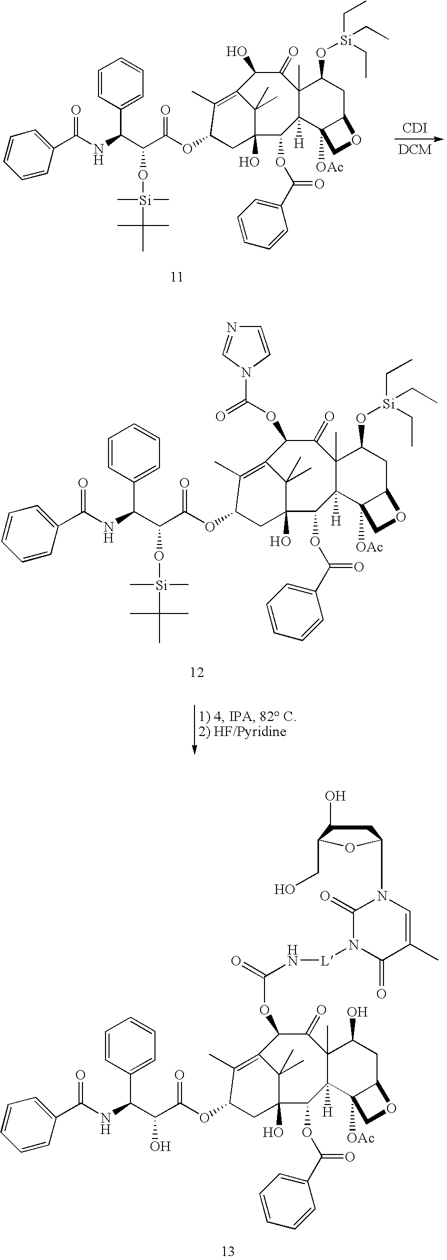

i) Preparation of 2′-O-(tert-butlyldimethylsilyl)-7-O-(tri-ethylsilyl)-10-O-deacetyl, 10-O-(imidazoylcarbonyl)-paclitaxel (12)

To 2′-O-(tert-butyldimethylsilyl)-7-O-(triethylsilyl)-10-O-deacetyl paclitaxel (11, 845 mg, 0.812 mmol), prepared according to the procedure described in Datta, A.; Hepperle, M. I. G. J. Org. Chem. (1995) 60: 761, in anhydrous DCM (6 mL) is added carbonyldiimidazole (530 mg, 400 mol %). The reaction mixture is allowed to stir for 16 hours at room temperature under nitrogen atmosphere then extracted with water (5 mL). The organic layer is dried over sodium sulfate, filtered and concentrated to give 890 mg of the title compound 12 which is subsequently used without purification.

ii) Reaction of 2′-O-(tert-butlyldimethylsilyl)-7-O-(tri-ethylsilyl)-10-O-deacetyl, 10-O-(imidazoylcarbonyl)-paclitaxel) with Thymidine-Linker-Amines

To 2′-O-(tert-butlyldimethylsilyl)-7-O-(tri-ethylsilyl)-10-O-deacetyl, 10-O-(imidazoylcarbonyl)-paclitaxel (12, 100 mol %), dissolved in anhydrous isopropyl alcohol is added thymidine-linker-amine 4 (500 mol %). The reaction mixture was stirred under reflux for 16 hours. The volatiles are then removed in vacuo and the resulting residue is re-dissolved in DCM. The organic solution is then extracted with water and dried over sodium sulfate. After filtration and evaporation of the volatiles the residue is desilylated following the procedure in Ojima, I. et al. J. Med. Chem. (1997), 40: 267. The residue so obtained is purified by preparative RP-HPLC (Method A). Fractions containing the appropriate mass, as determined by analytical HPLC-MS (Method B) are pooled and CH₃CN removed under reduced pressure. The remaining aqueous mixture is then lyophilized obtaining the desired paclitaxel-10-deacetyl, 10-oxycarbonylamino-linker-thymidine of general structure 13.

e. Preparation of a Doxorubicin-Linker-Thymidine with Alkyl Linker Attachment at C3′-N on Doxorubicin and Linker Attachment at N3′-Thymidine 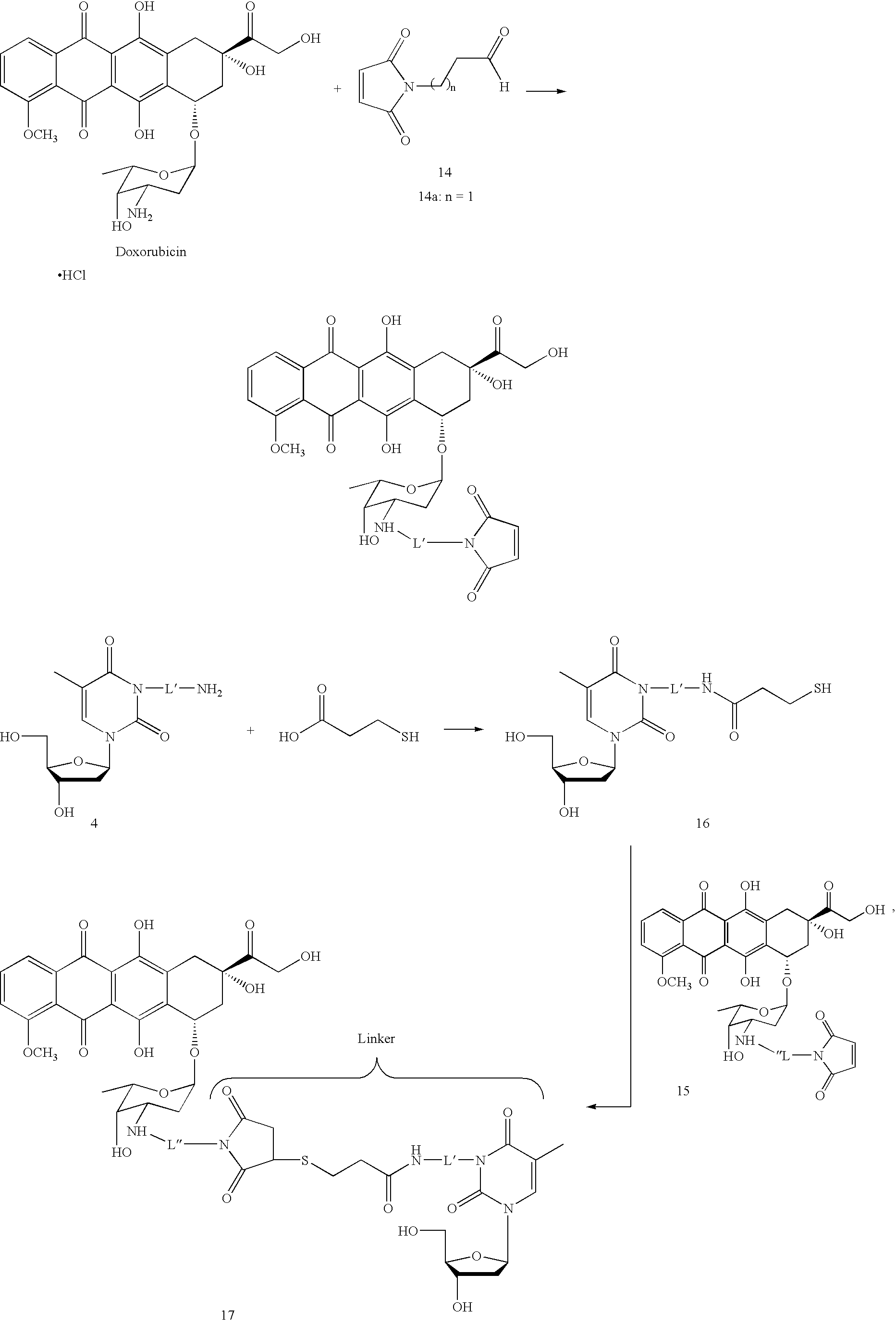
where L′ and L″ represent atoms between the first functionality and the second functionality of the linker moiety.

i) Preparation of a Thymidine-Linker-NHCOCH₂CH₂SH (16) Suitable for Reaction with the Alkyl Anthracycline-Maleimide Intermediate

To a thymidine-linker-NH₂ 4 (100 mol %) in DMF prepared according to the procedure described herein is added BOP (100 mol %), DIEA (400 mol %) and HOOCCH₂CH₂SH (100 mol %). The reaction mixture is stirred for 30 mm whereupon DMF is removed in vacuo. The crude is purified by silica gel P-TLC eluted with DCM/CH₃OH (9:1) to yield a thiol containing thymidine of general structure 16.

ii) Preparation of 3-(2,5-dioxo-2,5-dihydropyrrol-1-yl)-propionaldehyde (14)

To 1-(3-hydroxypropyl)-1H-pyrrole-2,5-dione dissolved in 5 mL DCM. DMP (15% wt in DCM) is added in one portion. After stirring the mixture for 2 h, 2-propanol is added followed by stirring for an additional 30 min. The resulting solution is filtered through a silica gel pad eluted with EtOAc, and the filtrate is concentrated. The crude product is purified by silica gel chromatography eluting with EtOAc/Hexane (2/1) to provide 3-(2,5-Dioxo-2,5-dihydro-pyrrol-1-yl)-propionaldehyde (14a) which is used immediately.

iii) Preparation of an Anthracycline-Maleimide Intermediate with N-alkyl Attached to 3′-N of the Anthracycline

To a stirred solution of doxorubicin hydrochloride, an aldehyde-maleimide intermediate (14, 200-300 mol %) and glacial AcOH (195 mol %) in CH₃CN/H₂O (2:1) is added a 1 M solution of NaCNBH₃ in THF (0.33 mol %). The mixture is stirred under nitrogen atmosphere in the dark at RT for 1 h. The solution is then concentrated under vacuum to give a residue which is diluted with an aqueous 5% NaHCO₃ solution and extracted with DCM. Concentration of the organic solution and purification of the resulting residue by silica gel chromatography eluting with DCM/CH₃OH (20:1) provided the anthracycline-maleimide intermediate of general structure 15.

iv) Preparation of a Doxorubicin-Linker-Thymidine with Alkyl Linker Attachment at C3′-N on Doxorubicin and Linker Attachment at N3′-Thymidine

To a DCM/CH₃OH (9:1) solution of 15 (100 mol %) is added a thiol containing thymidine of general structure 16 (100 mol %). The mixture is stirred under nitrogen atmosphere in the dark for 30 min. The solvent is removed in vacuo and the resulting crude residue is dissolved into by DMSO and purified on a preparative RP-HPLC C-18 reversed phase column for purification (Method A). Fractions containing the appropriate mass, as determined by analytical HPLC-MS (Method B), are pooled and CH₃CN is removed under reduced pressure or N₂ stream followed by lyophilization to give the anthracycline-linker-thymidine conjugate of general structure 17.

Several conjugates have been prepared by following the procedures described herein and slight modifications thereof. Tables 4-6 provide mass spectroscopy (Electrospray) data for exemplary conjugates.

TABLE 4

| | | | | | | Retention |
|---|---|---|---|---|---|---|
| | | | | | | Time (min) |
| | | | | | | (HPLC |
| Systematic Name | Formula | Mol Weight | Purity | MS Expected | MS Observed | Method B) |
| PXL-7Ca-ALKa(9)-N3-THY | C66H79N5O21 | 1278.371 | >95% | 1279(M + H) | 1279(M + H) | 8.39* |
| PXL-7Ca-ALK(6)-N3-THY | C64H76N4O20 | 1221.3192 | >95% | 1222(M + H) | 1222(M + H) | 6.22 |
| PXL-7Ca-ALK(6)-N3-5′-deoxy-THY | C64H76N4O19 | 1205.3198 | 93% | 1206 (M + H) | 1206 (M + H) | 6.94 |
| PXL-7Ca-ALK(6)-N3-αTHY | C64H76N4O20 | 1221.3192 | 98% | 1222 (M + H) | 1222 (M + H) | 6.22 |
| PXL-7Ca-ALK(6)-N3-5,6-dihydro-THY | C64H78N4O20 | 1223.335 | 96% | 1224 (M + H) | 1224 (M + H) | 6.24 |
| PXL-7Ca-PEG(11)-N3-THY | C66H80N4O23 | 1297.371 | 93% | 1298 (M + H) | 1298 (M + H) | 6.18 |
| PXL-10Ca-ALK(6)-N3-THY | C62H74N4O19 | 1179.282 | >95% | 1179 (M + H) | 1179 (M + H) | 5.87 |
| PXL-10Ca-ALK(6)-N3-5,6-dihydro-THY | C62H76N4O19 | 1181.2978 | 93% | 1181 (M + H) | 1181 (M + H) | 5.73 |
| PXL-10Ca-ALK(6)-N3-αTHY | C62H74N4O19 | 1179.282 | >95% | 1179 (M + H) | 1179 (M + H) | 5.82 |
| PXL-10Ca-PEG(5)-N3-THY | C60H70N4O20 | 1167.2278 | 99% | 1167 (M + H) | 1167 (M + H) | 5.57 |
| PXL-10Ca-PEG(5)-N3-αTHY | C60H70N4O20 | 1167.2278 | >95% | 1167 (M + H) | 1167 (M + H) | 5.55 |
| PXL-7Ca-PEG(5)-N3-THY | C62H72N4O21 | 1209.265 | 97% | 1209 (M + H) | 1209 (M + H) | 5.89 |
| PXL-10Ca-PEG(11)-N3-THY | C64H78N4O22 | 1255.3338 | 99% | 1256 (M + H) | 1256 (M + H) | 5.57 |
| PXL-10Ca-PEG(11)-N3-αTHY | C64H78N4O22 | 1255.3338 | >95% | 1256 (M + H) | 1256 (M + H) | 5.58 |
| PXL-7Ca-ALK(6)-N3-5′-phospho-THY | C64H77N4O23P | 1301.2991 | 94% | 1302 (M + H) | 1302 (M + H) | 5.97 |
| PXL-10Ca-ALK(3)-N3-THY | C59H68N4O19 | 1137.2016 | 95% | 1137 (M + H) | 1137 (M + H) | 5.53 |
| PXL-7Ca-ALK(3)-N3-THY | C61H70N4O20 | 1179.2388 | 94% | 1179 (M + H) | 1179 (M + H) | 5.84 |
| PXL-7Ca-ALK(4)-N3-THY | C62H72N4O20 | 1193.2656 | 99% | 1193 (M + H) | 1193 (M + H) | 5.92 |
| PXL-7Ca-ALK(5)-N3-THY | C63H74N4O20 | 1207.2924 | 99% | 1208 (M + H) | 1208 (M + H) | 6.07 |
| PXL-10Ca-ALK(5)-N3-THY | C61H72N4O19 | 1165.2552 | 95% | 1165 (M + H) | 1165 (M + H) | 5.75 |
| PXL-7Ca-ALK(8)-N3-THY | C66H80N4O20 | 1249.3728 | 99% | 1250 (M + H) | 1250 (M + H) | 6.62 |

TABLE 5

| | | | | | | Retention |
|---|---|---|---|---|---|---|
| | | | | | | Time |
| | | | | | | (min) |
| | | | | MS | MS | (HPLC |
| Systematic Name | Formula | Mol Weight | Purity | Expected | Observed | Method B) |
| VBL-3Am-ALK(6)-N3-THY | C59H79N7O12 | 1078.3128 | >95% | 1079(M + H) | 1079(M + H) | 6.39* |
| VBL-3Am-ALK(6)-N3-DeTHY | C59H79N7O11 | 1062.3134 | 98% | 1063(M + H) | 1063(M + H) | 4.32 |
| VBL-3Am-PEGa(14)-N3-THY | C63H86N8O16 | 1211.4164 | >95% | 1212(M + H) | 1212(M + H) | 6.17 |
| VBL-3Am-ALKa(6)-N3-THY | C58H76N8O13 | 1093.2842 | >95% | 1094(M + H) | 1094(M + H) | 6.01 |
| VBL-3Am-PEG(11)-N3-THY | C61H83N7O15 | 1154.3646 | 94 | 1155 (M + H) | 1155 (M + H) | 3.88 |
| VBL-3Am-PEG(5)-N3-THY | C57H75N7O13 | 1066.2586 | 99 | 1067 (M + H) | 1067 (M + H) | 3.81 |
| VBL-3Am-ALK(6)-N3-PhTHY | C59H80N7O15P | 1158.2927 | 95% | 1159 (M + H) | 1159 (M + H) | 3.97 |
| VBL-3Am-ALK(3)-N3-THY | C56H73N7O12 | 1036.2324 | 94% | 1037 (M + H) | 1037 (M + H) | 3.83 |
| VBL-3Am-ALK(4)-N3-THY | C57H75N7O12 | 1050.2592 | 96% | 1051 (M + H) | 1051 (M + H) | 3.77 |

TABLE 6

| | | | | | | Retention |
|---|---|---|---|---|---|---|
| | | | | | | Time (min) |
| | | | | MS | MS | (HPLC |
| Systematic Name | Formula | Mol Weight | Purity | Expected | Observed | Method B) |
| DOX-3′ALK-MALa(17)-N3-THY | C53H67N5O19S | 1110.1944 | NA | | NA | NA |
| DOX-3′Alk-[MALaPEG](22)-N3-THY × TFA | C57H72F3N5O24S | 1300.2701 | 94% | | NA | NA |

D. Formulation of Pharmaceutical Compositions

The pharmaceutical compositions provided herein contain therapeutically effective amounts of one or more of conjugates provided herein that are useful in the prevention, treatment, or amelioration of one or more of the symptoms of ACAMPS conditions. Such conditions include, but are not limited to, cancer, coronary restenosis, osteoporosis and syndromes characterized by chronic inflammation and/or autoimmunity. Examples of chronic inflammation and/or autoimmune diseases include but are not limited to rheumatoid arthritis and other forms of arthritis, asthma, psoriasis, inflammatory bowel disease, systemic lupus erythematosus, systemic dermatomyositis, inflammatory ophthalmic diseases, autoimmune hematologic disorders, multiple sclerosis, vasculitis, idiopathic nephrotic syndrome, transplant rejection and graft versus host disease.

The compositions contain one or more conjugates provided herein. The conjugates are preferably formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the conjugates described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more conjugates or pharmaceutically acceptable derivatives is (are) mixed with a suitable pharmaceutical carrier or vehicle. The conjugates may be derivatized as the corresponding salts, esters, enol ethers or esters, acids, bases, solvates, hydrates or prodrugs prior to formulation, as described above. The concentrations of the conjugates in the compositions are effective for delivery of an amount, upon administration, that treats, prevents, or ameliorates one or more of the symptoms of conditions associated with ACAMPS. Such conditions include, but are not limited to, cancer, coronary restenosis, osteoporosis and syndromes characterized by chronic inflammation and/or autoimmunity.

Typically, the compositions are formulated for single dosage administration. To formulate a composition, the weight fraction of conjugate is dissolved, suspended, dispersed or otherwise mixed in a selected vehicle at an effective concentration such that the treated condition is relieved or ameliorated. Pharmaceutical carriers or vehicles suitable for administration of the conjugates provided herein include any such carriers known to those skilled in the art to be suitable for the particular mode of administration.

In addition, the conjugates may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active conjugate is included in the pharmaceutically acceptable carrier in an amount sufficient to exert a therapeutically useful effect in the absence of undesirable side effects on the patient treated. The therapeutically effective concentration may be determined empirically by testing the conjugates in in vitro and in vivo systems described herein and then extrapolated therefrom for dosages for humans.

The concentration of active conjugate in the pharmaceutical composition will depend on absorption, inactivation and excretion rates of the active conjugate, the physicochemical characteristics of the conjugate, the dosage schedule, and amount administered as well as other factors known to those of skill in the art. For example, the amount that is delivered is sufficient to ameliorate one or more of the symptoms of diseases or disorders associated with ACAMPS condition as described herein.

Typically a therapeutically effective dosage should produce a serum concentration of active ingredient of from about 0.1 ng/ml to about 50-100 μg/ml. The pharmaceutical compositions typically should provide a dosage of from about 0.001 mg to about 2000 mg of conjugate per kilogram of body weight per day. Pharmaceutical dosage unit forms are prepared to provide from about 1 mg to about 1000 mg and preferably from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Pharmaceutically acceptable derivatives include acids, bases, enol ethers and esters, salts, esters, hydrates, solvates and prodrug forms. The derivative is selected such that its pharmacokinetic properties are superior to the corresponding neutral conjugate.

Thus, effective concentrations or amounts of one or more of the conjugates described herein or pharmaceutically acceptable derivatives thereof are mixed with a suitable pharmaceutical carrier or vehicle for systemic, topical or local administration to form pharmaceutical compositions. Conjugates are included in an amount effective for ameliorating one or more symptoms of, or for treating or preventing diseases or disorders associated with ACAMPS condition as described herein. The concentration of active conjugate in the composition will depend on absorption, inactivation, excretion rates of the active conjugate, the dosage schedule, amount administered, particular formulation as well as other factors known to those of skill in the art.

The compositions are intended to be administered by a suitable route, including orally, parenterally, rectally, topically and locally. For oral administration, capsules and tablets are presently preferred. The compositions are in liquid, semi-liquid or solid form and are formulated in a manner suitable for each route of administration. Preferred modes of administration include parenteral and oral modes of administration. Oral administration is presently most preferred.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the conjugates exhibit insufficient solubility, methods for solubilizing conjugates may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the conjugate(s), the resulting mixture may be a solution, suspension, emulsion or the like. The form of the resulting mixture depends upon a number of factors, including the intended mode of administration and the solubility of the conjugate in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the conjugates or pharmaceutically acceptable derivatives thereof. The pharmaceutically therapeutically active conjugates and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active conjugate sufficient to produce the desired therapeutic effect, in association with the required pharmaceutical carrier, vehicle or diluent. Examples of unit-dose forms include ampules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active conjugate as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active conjugate in an amount sufficient to alleviate the symptoms of the treated subject.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100% active ingredient, preferably 0.1-85%, typically 75-95%.

The active conjugates or pharmaceutically acceptable derivatives may be prepared with carriers that protect the conjugate against rapid elimination from the body, such as time release formulations or coatings.

The compositions may include other active conjugates to obtain desired combinations of properties. The conjugates provided herein, or pharmaceutically acceptable derivatives thereof as described herein, may also be advantageously administered for therapeutic or prophylactic purposes together with another pharmacological agent known in the general art to be of value in treating one or more of the diseases or medical conditions referred to hereinabove, such as diseases or disorders associated with ACAMPS. It is to be understood that such combination therapy constitutes a further aspect of the compositions and methods of treatment provided herein.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, preferably capsules or tablets. The tablets, pills, capsules, troches and the like can contain any of the following ingredients, or conjugates of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the conjugate could be provided in a composition that protects it from the acidic environment of the stomach. For example, the composition can be formulated in an enteric coating that maintains its integrity in the stomach and releases the active conjugate in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The conjugates can also be administered as a component of an elixir, suspension, syrup, wafer, sprinkle, chewing gum or the like. A syrup may contain, in addition to the active conjugates, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a conjugate or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active conjugate or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those containing a conjugate provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

2. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. Briefly, a conjugate provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The conjugate diffuses through the outer polymeric membrane in a release rate controlling step. The percentage of active conjugate contained in such parenteral compositions is highly dependent on the specific nature thereof, as well as the activity of the conjugate and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active conjugate is adjusted so that an injection provides an effective amount to produce the desired pharmacological effect. The exact dose depends on the age, weight and condition of the patient or animal as is known in the art.

The unit-dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active conjugate is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, preferably more than 1% w/w of the active conjugate to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The conjugate may be suspended in micronized or other suitable form or may be derivatized to produce a more soluble active product or to produce a prodrug. The form of the resulting mixture depends upon a number of factors, including the intended mode of administration and the solubility of the conjugate in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a conjugate provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg, preferably 100-500 mg) or multiple dosages of the conjugate. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, preferably 5-35 mg, more preferably about 9-30 mg of lyophilized powder, is added per mL of sterile water or other suitable carrier. The precise amount depends upon the selected conjugate. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The conjugates or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The conjugates may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active conjugate alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

6. Articles of Manufacture

The conjugates or pharmaceutically acceptable derivatives can be packaged as articles of manufacture containing packaging material, a conjugate or pharmaceutically acceptable derivative thereof provided herein, which is used for treatment, prevention or amelioration of one or more symptoms associated with ACAMPS condition, and a label that indicates that the conjugate or pharmaceutically acceptable derivative thereof is used for treatment, prevention or amelioration of one or more symptoms associated with ACAMPS condition.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the conjugates and compositions provided herein are contemplated as are a variety of treatments for any disorder associated with ACAMPS conditions.

E. Evaluation of the Activity of the Conjugates

Standard physiological, pharmacological and biochemical procedures are available for testing the conjugates to identify those that possess biological activity, including kinase activity. In vitro and in vivo assays that can be used to evaluate biological activity, such as cytotoxicity, which will depend upon the therapeutic agent being used in the conjugate.

Exemplary assays are discussed briefly below with reference to cytotoxic conjugates (see, also, Examples). It is understood that the particular activity assayed will depend upon the conjugated therapeutic agent.

1. Kinase Activity

Thymidine kinase, viral thymidine kinase, TK-1 deoxycytidine kinase, and deoxyguanosine kinase activities are determined by subjecting a first end of a linker used in synthesizing linker-substrate constructs to a first test. The first test may involve observing ADP formation, an obligatory co-product of phospho group transfer from ATP which is catalyzed by the kinase to the C5′ hydroxyl group or its equivalent in the nucleoside or nucleoside analog. Formation of ADP is followed by a coupled enzyme assay well known in the art. ADP, formed from kinase phosphorylation, is used by pyruvate kinase to generate pyruvate from phosphoenolpyruvate which in turn is converted to lactate by lactate dehydrogenase. The lactate results in the consumption of NADH which is followed spectrophotometrically. The rate of nucleoside phosphorylation is then directly related to the rate of decrease in the observed NADH signal.

Another test may involve monitoring the consumption of ATP. For example, ATP concentrations at time 0 or after 4 hour incubation may be monitored by luciferase reaction (PKLight™ kit obtained from Cambrex Corporation, One Meadowlands Plaza, East Rutherford, N.J. 07073), which generate a luminescence readout in the presence of ATP. Assays are initiated by mixing a kinase and a drug conjugate in the presence of 40 μM ATP. After 4 hour of incubation at 30° C., PKLight™ reagent is added and mixed well, and luminescence readout measured. The rate of drug conjugate phosphorylation is then directly related to the rate of decrease in the observed luminescence. Based on the first test, linkers of appropriate lengths and substrate with an effective amount of kinase activity which may be expected to be retained in the drug conjugate may be found. For paclitaxel drug conjugates BSA is employed in the first test to prevent drug conjugate aggregation.

2. Tubulin Polymerization Assay

Drug-linker constructs may further be screened using functional assays predictive of biological activity. In one example, microtubule stabilization for paclitaxel drug linker constructs or microtubule disruption by vinblastine drug-linker constructs is determined with a tubulin polymerization assay (Barron, et al., Anal. Biochem. (2003) 315: 49-56). Tubulin assembly or inhibition thereof may be monitored by fluorescence using the CytoDYNAMIX Screen™ 10 kit available from Cytoskeleton (1830 S. Acoma St., Denver, Colo.). The kit is based upon an increase in quantum yield of florescence upon binding of a fluorophore to tubulin and microtubules and a 10× difference in affinity for microtubules compared to tubulin. Emission is monitored at 405 nm with excitation at 360 nm. The compounds such as paclitaxel which enhance tubulin assembly will therefore give an increase in emission whereas compounds such as vinblastine which inhibit tubulin assembly will give a decrease in emission. Tubulin assembly or inhibition may also be monitored by light scattering which is approximated by the apparent absorption at 350 nm. For paclitaxel drug conjugates BSA is employed to prevent aggregation and glycerol, which is a tubulin polymerization enhancer, is omitted from the kit to increase the signal to noise ratio.

In certain embodiments, activity of doxorubicin conjugates was assayed by monitoring alteration in the ability of Topoisomerase II, by electrophoresis, to catalyze the formation of relaxed conformation DNA from a super-coiled plasmid. The more active a conjugate is at a particular concentration the less relaxed conformation DNA is produced by the action of Topoisomerase II.

In another example, a functional assay for camptothecin drug-linker constructs depends on inhibition of Topoisomerase I binding to DNA. In another example, a functional assay for camptothecin drug-linker constructs depends on inhibition of Topoisomerase I binding to DNA (Demarquay, Anti-Cancer Drugs (2001) 12: 9-19).

For each type of functional assay, the enzyme (kinase) and biochemical microtubule polymerization results for all synthetic lots of each compound were combined and analyzed using GraphPad Prism® software to generate the mean±SD.

For each specific cell-based assay, results from all assays carried out with all synthetic lots of each compound were combined and analyzed using Graph Pad Prism software® to generate the mean±SD. Outliers (<7% of the total dataset) were identified and removed prior to analysis using the method of Hoaglin et al., J. Amer. Statistical Assoc., 81, 991-999, 1986. Compounds were tested between five and twenty times (in triplicate) in each assay. The significance of differences between the cytotoxic EC₅₀s of each compound against normal and tumor cell types (cytotoxic selectivity index) was determined with an unpaired t test (95% confidence interval) using GraphPad Prism® software.

Tables 7-9 provide results for cytotoxicity, kinase activity and Topoisomerase II assay for exemplary conjugates and their parent drugs provided herein. Detailed procedures for conducting the assays are provided in the Examples section. The conjugates provided herein typically exhibit higher cytotoxic selectivity index in tumor cells as compared to their parent drugs. The conjugates are more selective for the tumor cells than the normal cells.

Average EC50 (“EC50-AVG”) for is provided as follows: A<0.1 μM, B=0.02-0.1 μM, C>0.1-1.0 μM and N/A=not available or inactive. Average TK1 kinase activity is provided as follows: A<20, B=20-40 C>40 and N/A=not available or inactive. Average MPA activity is provided as follows: A<60, B=60-100 C>100 and N/A=not available or inactive.

TABLE 7

| | Ave. | | | | | | | |
|---|---|---|---|---|---|---|---|---|
| | TK1 | Ave. | MCF7 | MCF7 | HT29 | HT29 | HUVEC | HFF |
| | Kinase | MPA | (EC50 Ave) | (EC50 Ave) | (EC50 Ave) | (EC50 Ave) | (EC50 Ave) | (EC50 Ave) |
| Systematic Name | Act. | Act | ML | SA | ML | SA | ML | ML |
| Paclitaxel (PXL) | N/A | C | A | A | A | A | A | A |
| PXL-7Ca-ALKa(9)-N3- | N/A | B | N/A | N/A | N/A | N/A | C | C |
| THY | | | | | | | | |
| PXL-7Ca-ALK(6)-N3-THY | A | B | A | A | B | B | B | B |
| PXL-7Ca-ALK(6)-N3- | A | A | B | A | B | A | B | B |
| DeTHY | | | | | | | | |
| PXL-7Ca-ALK(6)-N3- | N/A | B | C | B | C | C | C | C |
| αTHY | | | | | | | | |
| PXL-7Ca-ALK(6)-N3- | A | B | A | A | B | A | A | A |
| H2THY | | | | | | | | |
| PXL-7Ca-PEG(11)-N3-THY | B | A | C | A | C | A | A | A |
| PXL-10Ca-ALK(6)-N3- | A | A | B | A | B | A | B | C |
| THY | | | | | | | | |
| PXL-10Ca-Alk(6)-N3- | A | B | B | A | A | A | A | A |
| H2THY | | | | | | | | |
| PXL-10Ca-ALK(6)-N3- | A | B | B | A | B | A | B | B |
| αTHY | | | | | | | | |
| PXL-10Ca-PEG(5)-N3-THY | B | C | B | N/A | B | A | C | C |
| PXL-10Ca-PEG(5)-N3- | A | B | B | A | B | A | B | C |
| αTHY | | | | | | | | |
| PXL-7Ca-PEG(5)-N3-THY | A | A | C | A | C | A | A | A |
| PXL-10Ca-PEG(11)-N3- | B | C | A | N/A | A | B | A | C |
| THY | | | | | | | | |
| PXL-10Ca-PEG(11)-N3- | A | B | C | N/A | C | N/A | C | C |
| αTHY | | | | | | | | |
| PXL-7Ca-ALK(6)-N3- | N/A | B | C | N/A | C | N/A | C | C |
| PhTHY | | | | | | | | |
| PXL-10Ca-Alk(3)-N3-THY | B | C | B | N/A | B | N/A | A | B |
| PXL-7Ca-ALK(3)-N3-THY | B | B | C | N/A | C | N/A | C | C |
| PXL-7Ca-ALK(4)-N3-THY | C | B | C | N/A | C | N/A | C | C |
| PXL-7Ca-ALK(5)-N3-THY | A | B | C | N/A | C | N/A | C | C |
| PXL-10Ca-ALK(5)-N3- | B | B | B | N/A | B | N/A | B | B |
| THY | | | | | | | | |
| PXL-7Ca-ALK(8)-N3-THY | A | B | A | N/A | A | N/A | A | B |
| Vinblastine (VBL) | A | C | A | A | A | A | A | A |
| VBL-3Am-ALK(6)-N3- | A | B | B | A | A | A | A | A |
| THY | | | | | | | | |
| VBL-3Am-ALK(6)-N3- | A | B | A | A | A | A | A | A |
| DeTHY | | | | | | | | |
| VBL-3Am-ALK(6)-N3- | A | B | A | A | A | A | A | A |
| DeTHY | | | | | | | | |
| VBL-3Am-PEGa(14)-N3- | A | C | A | A | A | A | B | B |
| THY | | | | | | | | |
| VBL-3Am-ALKa(6)-N3- | A | C | B | A | C | A | B | B |
| THY | | | | | | | | |
| VBL-3Am-PEG(11)-N3- | B | A | A | A | A | B | A | A |
| THY | | | | | | | | |
| VBL-3Am-PEG(5)-N3-THY | A | C | A | A | A | A | A | A |
| VBL-3Am-ALK(6)-N3- | N/A | C | A | N/A | A | N/A | A | A |
| PhTHY | | | | | | | | |
| VBL-3Am-ALK(3)-N3- | A | C | B | C | B | A | A | A |
| THY | | | | | | | | |
| VBL-3Am-ALK(4)-N3- | B | C | B | A | N/A | A | A | B |
| THY | | | | | | | | |
| Doxorubicin (DOX) | A | C | A | N/A | B | N/A | A | A |
| DOX-3′ALK-MALa(17)- | A | A | C | N/A | N/A | N/A | C | C |
| N3-THY | | | | | | | | |
| DOX-3′Alk- | A | N/A | N/A | N/A | N/A | N/A | C | N/A |
| [MALaPEG](22)-N3-THY | | | | | | | | |
| Vinblastine (VBL) | A | N/A | N/A | N/A | N/A | N/A | C | N/A |

TABLE 8
PACLITAXEL NON-TARGETED DERIVATIVES

| | Ave. | | MCF7 | MCF7 | | HT29 | HT29 | HT29 | HUVEC | HFF |
|---|---|---|---|---|---|---|---|---|---|---|
| | TK1 | Ave. | (EC50 | (EC50 | MCF7 | (EC50 | (EC50 | (EC50 | (EC50 | (EC50 |
| | Kinase | MPA | Ave) | Ave) | (EC50) | Ave) | Ave) | Ave) | Ave) | Ave) |
| Systematic Name | Act | Act | ML | SA | SSA | ML | SA | SSA | ML | ML |
| Paclitaxel (PXL) | A | C | A | A | A | A | A | A | A | A |
| PXL-7Es-ALK(5)-NH2 | — | A | C | A | A | C | A | A | A | A |
| PXL-7Ca-ALK(6)-NH2 | — | A | C | A | A | A | A | A | A | a |
| PXL-7Ca-ALK(6)-Phospho(OPh, N-Ala) | — | A | B | A | A | A | A | A | A | A |
| PXL-7Ca-ALK(6)-diphenyl phosphoramidate | — | A | B | A | A | A | A | A | A | A |
| PXL-2′Alloc | — | A | A | A | A | A | A | A | A | A |
| PXL-10Es-Alk(6)-NH(Z) | — | A | A | A | A | A | A | A | A | A |
| 10 Deacetyl Taxol | — | — | B | A | A | A | A | A | A | A |
| PXL-10Es-ALK(5)-NH2 | — | B | A | A | A | A | A | A | A | A |
| PXL-10Ca-PEG(13)-NH(Z) | — | B | A | A | A | A | A | A | A | A |

TABLE 9
VINBLASTINE NON-TARGETED DERIVATIVES

| | Ave. | | MCF7 | MCF7 | | HT29 | HT29 | HT29 | HUVEC | HFF |
|---|---|---|---|---|---|---|---|---|---|---|
| | Tie2 | Ave. | (EC50 | (EC50 | MCF7 | (EC50 | (EC50 | (EC50 | (EC50 | (EC50 |
| | Kinase | MPA | Ave) | Ave) | (EC50) | Ave) | Ave) | Ave) | Ave) | Ave) |
| Systematic Name | Act | Act | ML | SA | SSA | ML | SA | SSA | ML | ML |
| Vinblastine (VBL) | — | C | A | A | A | A | A | A | A | A |
| VBL-3Am-ALK(8)-NH2 | — | B | A | A | A | A | A | A | A | A |
| VBL-3Am-ALK(6)-NH(B) | — | A | A | A | A | A | A | A | A | A |
| VBL-3Am-ALK(6)-NH2 | — | C | A | A | A | A | A | A | A | A |
| VBL-3Am-ALK(12)-NH(B) | — | A | B | A | A | C | A | A | A | A |
| VBL-3Am-ALK(12)-NH2 | — | B | A | A | A | A | A | A | A | A |
| VBL-3Am-PEG(11)-NH(B) | — | B | A | A | A | A | A | A | A | A |
| VBL-3Am-PEG(11)-NH2 | — | B | B | A | A | B | A | A | A | A |
| Desacetylvinblastine monohydrazine | — | C | A | A | A | A | A | A | A | A |
| Desacetyl vinblastine | — | C | A | A | A | A | A | A | A | A |

In certain embodiments, as demonstrated by a comparison of cytotoxic selectivity index for an exemplary conjugate and parent drug in tumors and normal cells, the conjugates show increase in the cytotoxic selectivity index of the conjugate for tumor cells as compared to the cytotoxic selectivity index of the parent drug:

| | | MCF-7 | |
|---|---|---|---|
| | HFF | Soft Agar | |
| | Monolayer | EC50 | HT-29 Soft Agar |
| | EC50 (nM) | (nM) | EC50 (nM) |
| Paclitaxel | 9 ± 5 | 6 ± 3 | 15 ± 2 |
| | (n = 20) | (n = 8) | (n = 6) |
| PXL-7Ca- | 457 ± 310 | 40 ± 41 | 120 ± 4 |
| ALK(6)- | (n = 16) | (n = 9) | (n = 5) |
| N3-Thy | | | |

The improvement in the cytotoxic selectivity index of the PXL-7Ca-ALK(6)-N-3-Thy conjugate as compared to the cytotoxic selectivity index of paclitaxel in exemplary cell lines, as illustrated by improved cytotoxic selectivity index index, is shown below:

Cytotoxic Selectivity Index

| | HFF/MCF7 | HFF/HT29 |
|---|---|---|
| Paclitaxel | 1.4 | 0.6 |
| PXL-7Ca- | 11.4 | 3.8 |
| ALK(6)-N3-Thy | | |

In certain embodiments, the conjugates show better serum stability as compared to the parent drug as demonstrated by an exemplary conjugate below:

| | | | | Relative Percent Remaining at | | | |
|---|---|---|---|---|---|---|---|
| | Initial Conc. (μM) | | | T_{1/2} (hr) | | | |
| Compound | 0 hr | 4 hr | 8 hr | 24 hr | 72 hr | | |
| Paclitaxel | 8.9 | 100 | 73 | 59 | 28 | <3.0 | 11 |
| PXL-7Ca- | 10 | 100 | 84 | 90 | 80 | 38 | 55 |
| ALK(6)-N3-Thy | | | | | | | |

One skilled in the art will appreciate that the assays described here may also be used to screen for direct substrate-drug conjugates (i.e., conjugates which contain no linker).

F. Methods of Use of the Conjugates and Compositions

Methods of use of the conjugates and compositions provided herein are also provided. The methods involve both in vitro and in vivo uses of the conjugates and compositions. The methods provided herein can be used for increasing drug efficiency. In certain embodiments, methods for treating conditions caused by undesirable chronic or aberrant cellular activation, migration, proliferation or survival (ACAMPS) are provided.

ACAMPS conditions are characterized by undesirable or aberrant activation, migration, proliferation or survival of tumor cells, endothelial cells, B cells, T cells, macrophages, granulocytes including neutrophils, eosinophils and basophils, monocytes, platelets, fibroblasts, other connective tissue cells, osteoblasts, osteoclasts and progenitors of many of these cell types. Examples of ACAMPS-related conditions include, but are not limited to, cancer, coronary restenosis, osteoporosis and syndromes characterized by chronic inflammation and/or autoimmunity. Examples of chronic inflammation and/or autoimmune diseases include but are not limited to rheumatoid arthritis and other forms of arthritis, asthma, psoriasis, inflammatory bowel disease, systemic lupus erythematosus, systemic dermatomyositis, inflammatory ophthalmic diseases, autoimmune hematologic disorders, multiple sclerosis, vasculitis, idiopathic nephrotic syndrome, transplant rejection and graft versus host disease.

Examples of cancers include, but are not limited to, non-small cell lung cancer, small cell lung cancer, head and neck squamous cancers, colorectal cancer, prostate cancer, and breast cancer, acute lymphocytic leukemia, adult acute myeloid leukemia, adult non-Hodgkin's lymphoma, brain tumors, cervical cancers, childhood cancers, childhood sarcoma, chronic lymphocytic leukemia, chronic myeloid leukemia, esophageal cancer, hairy cell leukemia, kidney cancer, liver cancer, multiple myeloma, neuroblastoma, oral cancer, pancreatic cancer, primary central nervous system lymphoma, skin cancer, and small-cell lung cancer. Childhood cancers amenable to treatment by the methods and with the compositions provided herein include, but are not limited to, brain stem glioma, cerebellar astrocytoma, cerebral astrocytoma, ependymoma, Ewing's sarcoma and family of tumors, germ cell tumor, Hodgkin's disease, ALL, AML, liver cancer, medulloblastoma, neuroblastoma, non-Hodgkin's lymphoma, osteosarcoma, malignant fibrous histiocytoma of bone, retinoblastoma, rhabdomyosarcoma, soft tissue sarcoma, supratentorial primitive neuroectodermal and pineal tumors, unusual childhood cancers, visual pathway and hypothalamic glioma, Wilms' tumor, and other childhood kidney tumors.

The methods and compositions provided can also be used to treat cancers that originated from or have metastasized to the bone, brain, breast, digestive and gastrointestinal systems, endocrine system, blood, lung, respiratory system, thorax, musculoskeletal system, and skin. The methods are generally applicable to all cancers but have particularly significant therapeutic benefit in the treatment of solid tumors. In certain embodiments, the solid tumors are characterized by extensive regions of hypoxic tissue. In certain embodiments, the drug moieties provided in Table 7 are used in the conjugates, which are used in treating particular types of cancer.

TABLE 7
Drug Selection

Paclitaxel (Taxane susceptible to MDR)
Breast, Lung, Prostate, Ovarian, Head & Neck, Esophageal, Bladder
Doxorubicin (Anthracycline/MDR)
Breast, Lung, Ovarian, Bladder, Hepatoma, Neuroblastoma, Lymphoma
Vinblastine (Vinca Alkaloid/MDR)
Breast, Lung, Prostate, Testicular, Renal, Lymphoma
Methotrexate (Antimetabolite)
Breast, Colorectal, Head & Neck, Leukemia, Lymphoma
Cisplatin (DNA Crosslinking Agent)
Lung, Ovarian, Head & Neck, Esophageal, Bladder, Lymphoma

G. Library and Screening Methods

In certain embodiments, the conjugates provided herein are produced using combinatorial methods to produce large libraries of potential conjugates. Methods for producing and screening combinatorial libraries of molecules are known in the art. The libraries of potential conjugates may then be screened for identification of a conjugate with the desired characteristics. Any convenient screening assay may be employed, where the particular screening assay may be known to those of skill in the art or developed in view of the specific molecule and property being studied.

For example, the libraries of potential conjugates may be screened for selectivity by comparing the conjugate activity in the target cell or tissue type to conjugate activity in cells or tissues in which drug activity is not desired. A selective conjugate will affect the target in the desired cells (e.g., cells involved in a disease process), but affect the target in undesired cells to a lesser extent or not at all. In another example, the libraries of potential conjugates may be screened for conjugates that exhibit enhanced drug efficiency as compared to the pharmacological activity of the unconjugated drug. For example, a more efficient drug will result in a desirable pharmacological response at a lower effective dose than a less efficient drug. In another example, a more efficient drug will have an improved cytotoxic selectivity index compared to a less efficient drug. In one example, the screening assay will involve observing the accumulation of the conjugate in the target system, in comparison to that of the unconjugated drug.

H. High Throughput Screening and Target Identification Methods for Kinase Substrate Trapping Using Drug-Linker-Conjugate Libraries

Provided herein is a broadly applicable method for specifically targeting and trapping non-specific drugs in cancer. In one embodiment, the conjugates provided are distinguished by retention of drug activity or a significant fraction thereof within the conjugate and therefore do not rely on release of free drug or activation of the drug by an intra-cellular protein. In one embodiment, the drug moiety and/or the substrate moiety in the conjugate can be present in a form of a pharmaceutically acceptable derivative that renders the conjugate biologically inactive. The inactive drug-substrate conjugate can be converted to the active drug-substrate conjugate under physiological conditions or by intracellular proteins without having the need to cleave the drug-substrate conjugate. In other embodiments, the conjugates are selectively targeted or trapped by cancer or viral infected cells due to phosphorylation of the substrate (e.g., nucleoside or nucleoside analog by a TK) whose activity is involved in the condition being treated.

Accumulation of the drug conjugate into the cancer or viral infected cell types will occur by pushing the equilibrium of passive diffusion towards the cancer or viral infected cells as a result of preferential trapping due to the higher kinase activity within these cell types. As a result, standard doses of the drug (in conjugate form) will produce enhanced efficacy, without an increase in undesirable side effects. In addition, the standard drug dose (in conjugate form) can be reduced, without loss of efficacy, but with a reduction in undesirable side effects. This allows for an increase in the duration of therapy, which is highly desirable in chronic disease settings. Finally, trapping or accumulation of drug conjugates by phosphorylation may prevent the efflux of cancer drugs, including vinca alkaloids, epipodophyllotoxins, taxanes/taxoids, and anthracyclines by the membrane transporter P-glycoprotein, preventing a major form of MDR.

Drugs such as paclitaxel and vinblastine can be prepared with a biotin moiety or fluorescent tag using procedures known in the art. (See, e.g., Guillemard et al., Anticancer Res. 1999 November-December; 19(6B): 5127-30; Dubois et al., Bioorg Med Chem. 1995 October; 3(10): 1357-68; Chatterjee et al., Biochemistry. 2002 Nov. 26; 41(47): 14010-8; Baloglu et al., Bioorg Med Chem Lett. 2001 Sep. 3; 11(17): 2249-52; Li et al., Biochemistry. 2000 Jan. 25; 39(3): 616-23; Rao et al., Bioorg Med Chem. 1998 November; 6(11): 2193-204; Bicamumpaka et al., Int J Mol Med. 1998 August; 2(2): 161-165; Sengupta et al., Biochemistry. 1997 Apr. 29; 36(17): 5179-84; Han et al., Biochemistry. 1996 Nov. 12; 35(45): 14173-83; Sengupta et al., Biochemistry. 1995 Sep. 19; 34(37): 11889-94). Substrate libraries can be conjugated to drugs (such as paclitaxel or vinblastine) which contain a biotin moiety or a fluorescent tag. A fluorescent drug (such as doxorubicin) can also be used. In the case of biotinylated conjugates, the libraries need not be purified. Large mixtures of conjugates can be incubated with various target cells (ACAMPS disease or normal), followed by removal of the extracellular medium, cell washing and isolation of phosphorylated (trapped or accumilated) conjugates from cell lysates using streptavidin or avidin affinity chromatography. Determination of the trapped or accumulated substrate by standard methods will identify a substrate of an overexpressed or activated kinase expressed in the diseased cell type (or disease-associated normal cell type). This provides a trapping or accumilation of the substrate candidate, which can then be used with the original drug or linked to other drugs and optimized.

Fluorescently tagged conjugates can be used with drug conjugate libraries that are produced in a “one conjugate per well” format. The libraries are incubated with tumor cells, endothelial cells or cells derived from any (ACAMPS) disease tissue, in a multi-well format, followed by washing and determination of well-associated fluorescence. Fluorescent drug conjugates that are retained to a high extent by diseased or other target cells represent novel drug candidates. Additionally, specificity can be assessed by comparing fluorescence uptake in the target cell to that in a normal cell type or one not associated with the disease of interest. The above methods are not limited to biotinylated or fluorescently tagged conjugates, but can be carried out with any tag or inherent property that facilitates purification or spectrophotometric visualization of conjugates specifically trapped or accumulated in target cells.

Since substrates are known for a large number of kinases, it is also possible to use these methods to identify new drug discovery (enzyme inhibition) targets for any ACAMPS disease. In certain embodiments, the methods can be used to identify an overexpressed or aberrantly activated kinase that has not previously been associated with a particular disease. In the instances where a biotinylated drug-substrate conjugate is employed, it could also be used to isolate the kinase in question from cell extracts via affinity chromatography. The kinase may be a previously identified or novel enzyme. The library and screening methods can be applied to small molecule or metabolic kinase substrates.

G. Combination Therapy

The conjugates provided herein may be administered as the sole active ingredient or in combination with other active ingredients. Other active ingredients that may be used in combination with the conjugates provided herein include but are not limited to, compounds known to treat ACAMPS conditions, anti-angiogenesis agents, anti-tumor agents, other cancer treatments and autoimmune agents. Such compounds include, in general, but are not limited to, alkylating agents, toxins, antiproliferative agents and tubulin binding agents. Classes of cytotoxic agents for use herein include, for example, the anthracycline family of drugs, the vinca drugs, the mitomycins, the bleomycins, the cytotoxic nucleosides, the pteridine family of drugs, diynenes, the maytansinoids, the epothilones, the taxanes and the podophyllotoxins.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### EXAMPLES

Abbreviations used: Cbz, benzyloxycarbonyl; CDI, 1,1′-carbonyldiimidazole; DCM, dichloromethane; DIEA, N,N-diisopropylethylamine; DMAP, 4-(dimethylamino)pyridine; DMF, N,N-dimethylformamide; IPA, isopropyl alcohol; MeOH, methanol; MS, mass spectroscopy; RP-HPLC, reversed phase high performance liquid chromatography; RT, room temperature; TEA, triethylamine; TFA, trifluoroacetic acid; Ts=Tosyl. Preparative RP-HPLC purification was conducted on YMC-Pack ODS-A columns (S-5 μM, 300×20 mm ID) with gradient elution between 0% B to 50% B or 0% B to 100% B (A=0.1% TFA in H₂O; B=0.05% TFA in CH₃CN) with gradient times of 10 min and a flow rate of 25 mL/min with UV 220 nm detection (Method A). Analytical HPLC-MS was conducted on a YMC Combi-Screen ODS-A column (S-5 μM, 50×4.6 mm ID) with gradient elution of %0 B to 100% B (A=0.05% TFA in H₂O; B=0.05% TFA in CH₃CN) with gradient times of 10 min and a flow rate of 3.5 mL/min with UV 220 nm and Electrospray MS detection (Method B).

### EXAMPLES

Syntheses of representative paclitaxel drug-linker constructs with carbamate linker to paclitaxel C10 are given in Examples 1-3 and Example 4. Example 6 provides a synthesis of a representative vinblastine drug-linker construct with amide linker to C3.

Abbreviations used: Cbz, benzyloxycarbonyl; CDI, 1,1′-carbonyldiimidazole; DCM, dichloromethane; DIEA, N,N-diisopropylethylamine; DMAP, 4-(dimethylamino)pyridine; DMF, N,N-dimethylformamide; IPA, isopropyl alcohol; MeOH, methanol; MS, mass spectroscopy; RP-HPLC, reversed phase high performance liquid chromatography; RT, room temperature; TEA, triethylamine; TFA, trifluoroacetic acid; Ts=Tosyl. Preparative RP-HPLC purification was conducted on YMC-Pack ODS-A columns (S-5 μM, 300×20 mm ID) with gradient elution between 0% B to 50% B or 0% B to 100% B (A=0.105% TFA in H₂₀; B=0.105% TFA in CH₃CN) with gradient times of 10 min and a flow rate of 25 mL/min with UV 220 nm detection (Method A). Analytical HPLC-MS was conducted on a YMC Combi-Screen ODS-A column (S-5 μM, 50×4.6 mm ID) with gradient elution of %0 B to 100% B (A=0.105% TFA in H₂O; B=0.105% TFA in CH₃CN) with gradient times of 10 min and a flow rate of 3.5 mL/min with UV 220 nm and Electrospray MS detection (Method B).

### Example 1

### Preparation of 2′-O-(tert-butyldimethylsilyl)-7-O-(triethylsilyl)-10-O-deacetyl-10-O-(carbonylimidazolyl)paclitaxel Paclitaxel-2′-(tert-butlyldimethylsilyl)-7-(triethylsilyl)-10-(deacetyl-carbonylimidazole) (2)

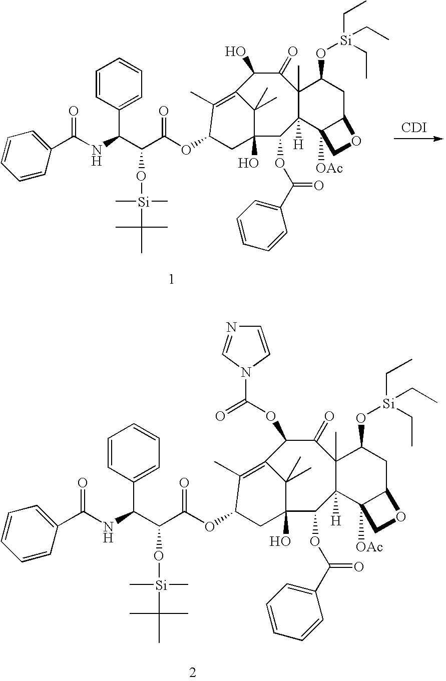

To 10-deacetyl-2′-O-(tert-butyldimethylsilyl)-7-O-(triethylsilyl)-10-O-deacetyl-paclitaxel (1, 845 mg, 0.81 mmol), prepared according to the procedure in Datta, A.; Hepperle, M. I. G. J. Org. Chem. (1995) 60: 761, in anhydrous DCM (6 mL) was added carbonyldiimidazole (530 mg, 400 mol %). The reaction mixture was allowed to stir for 16 hours at room temperature under nitrogen atmosphere then extracted with water (5 mL). The organic layer was dried over sodium sulfate, filtered and concentrated to give 890 mg (96% yield) of the title compound 2 which was subsequently used without purification. ¹H NMR (CDCl₃, 300 MHz) δ 8.26 (s, 1H), 8.18 (d, J=9 Hz, 2H), 7.77 (d, J=8 Hz, 2H), 7.53 (m, 11H), 7.14 (s, 1H), 7.09 (d, J=9 Hz, 1H), 6.59 (s, 1H), 6.32 (t, J=9 Hz, 1H), 5.78 (m, 2H), 5.02 (d, J=8 Hz, 1H), 4.72 (d, J=2 Hz, 1H), 4.56 (m, 1H), 4.38 (d, J=8 Hz, 1H), 4.25 (d, J=8 Hz, 1H), 3.88 (d, J=7 Hz, 1H), 2.62 (s, 2H), 2.45 (m, 1H), 2.2 (m, 1H), 2.18 (m, 1H), 1.98 (m, 1H), 1.81 (s, 1H), 1.78 (s, 3H), 1.62 (s, 3H), 1.32 (m, 3H), 1.22 (s, 3H), 0.95 (m, 6H), 0.83 (s, 9H), 0.62(m, 9H), 0.1 (s, 3H), −0.2 (s, 3H); Electrospray (LCMS) m/z 1134 (M+H⁺, C₆₁H₈₀N₃O₁₄Si₂ requires 1134); retention time=9.92 min. (1% to 99% B, Method B)

### Example 2

### Preparation of 2′-O-(tert-butyldimethylsilyl)-7-O-(triethylsilyl)-paclitaxel-10-O-(deacetyl)-10-O-{N-[(carbamoyl-3-[2-[2-[3-CBz-aminopropoxy]-ethoxy]-ethoxy]-propyl]-aminocarbonylamo}-paclitaxelPEG-N-Cbz-amide) (4)

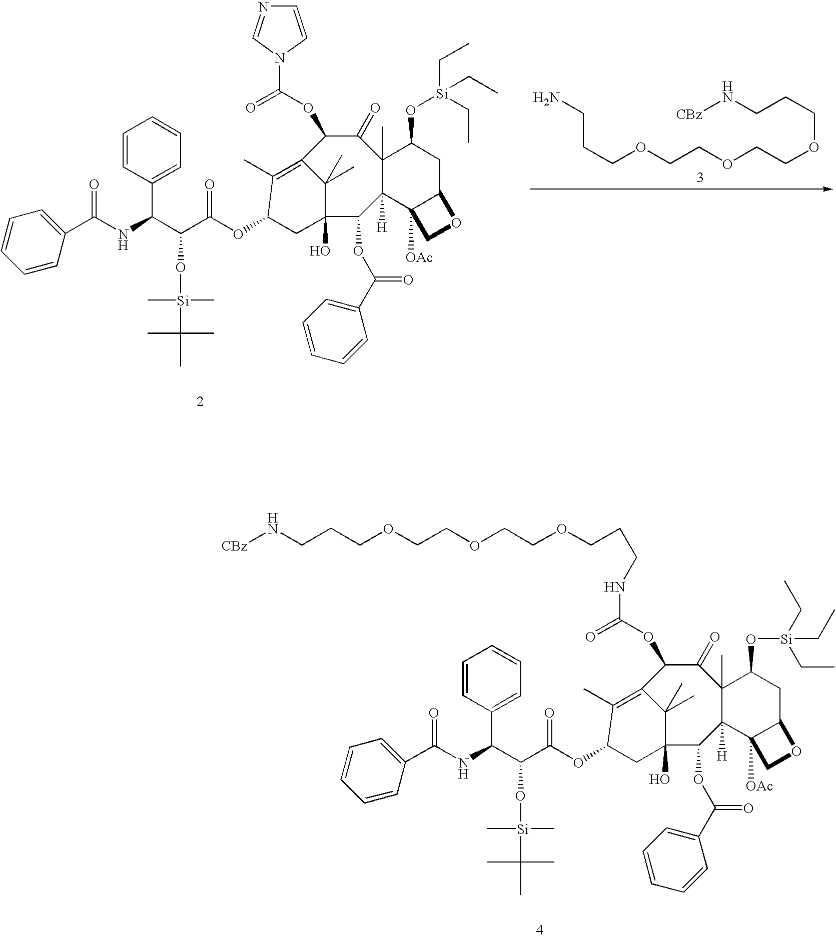

To 2′-O-(tert-butyldimethylsilyl)-7-O-(triethylsilyl)-10-deacetyl-10-O-(carbonylimidazolyl)-paclitaxelpaclitaxel-2′-(tert-butlyldimethylsilyl)-7-(triethylsilyl)-10-(deacetyl-carbonyl-imidazole) (2, 250 mg, 0.22 mmol), prepared according to Example 1, dissolved in anhydrous tert-butyl alcohol (5 mL) was added commercially available 3-[2-[2-[3-CBz-aminopropoxy]-ethoxy]-ethoxy]-propylamine mono-N-Cbz-amidoPEG-diamine (3, 398 mg, 510 mol %). The reaction mixture was stirred at 80° C. for 16 hours. The volatiles were then removed in vacuo and the resulting residue was re-dissolved in DCM (15 mL). The organic solution was then extracted with water (10 mL), dried over sodium sulfate, filtered and concentrated to give 284 mg of the title compound 4 which was subsequently used without purification. Electrospray (LCMS) m/z 1421 (M+H⁺, C₇₆H₁₀₆N₃O₁₉Si₂ requires 1421); retention time 10.49 min. (1% to 99% B, Method B);

### Example 3

### Preparation of Paclitaxel-10-O-deoxy-10-O-{N-[(carbamoyl-3-[2-[2-[3-aminopropoxy]-ethoxy]-ethoxy]-propyl]-aminocarbonylamo}-paclitaxelPEG-N-Cbz-amide) (5)

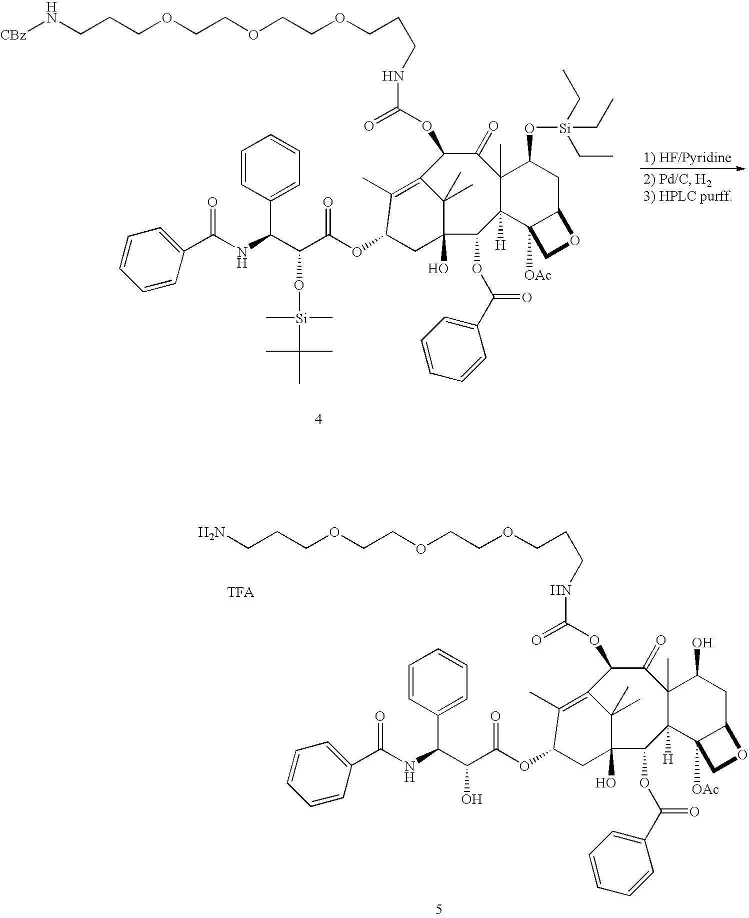

Compound 4 (284 mg, 0.2 mmol) prepared according to Example 2 was desylilated following the procedure in Ojima, I. et al. J. Med. Chem. (1997), 40: 267. The residue so obtained (225 mg) was dissolved in methanol (20, mL) whereupon 10 wt % palladium on carbon (100 mg) was added. The resulting mixture was stirred for 40 minutes under one atmosphere of H₂. The reaction mixture was filtered through Celite and concentrated under reduced pressure. The residue so obtained was purified by preparative RP-HPLC (Method A). Fractions containing the appropriate mass, as determined by analytical HPLC-MS (Method B) were pooled and CH₃CN removed under reduced pressure. The remaining aqueous mixture was then lyophilized obtaining 140 mg (55% overall yield) of the desired paclitaxel-linker construct 5 with PEG carbamate linker at C10. ¹H NMR (CD₃OD, 300 MHz) δ 8.38 (d, J=8 Hz, 1H), 8.14 (d, J=8 Hz, 2H), 7.89 (d, J=8 Hz, 2H), 7.45 (m, 11H), 6.29 (s, 1H), 6.19 (t, 1H), 5.66 (m, 2H), 5.03 (d, J=10 Hz, 2H), 4.76 (d, J=6 Hz, 2H), 4.35 (m, 1H), 4.22 (s, 2H), 3.85 (d, 1H), 3.60 (m, 8H), 3.12 (m, 2H), 2.50 (m, 1H), 2.40 (s, 3H), 2.26 (m, 1H), 2.19 (s, 2H), 1.94 (m, 4H), 1.82 (m, 4H), 1.68 (s, 2H), 1.18 (s, 6H); Electrospray (LCMS) m/z 1058 (M+H⁺, C₅₆H₇₂N₃O₁₇ requires 1058); retention time 5.07 min. (1% to 99% B, Method B).

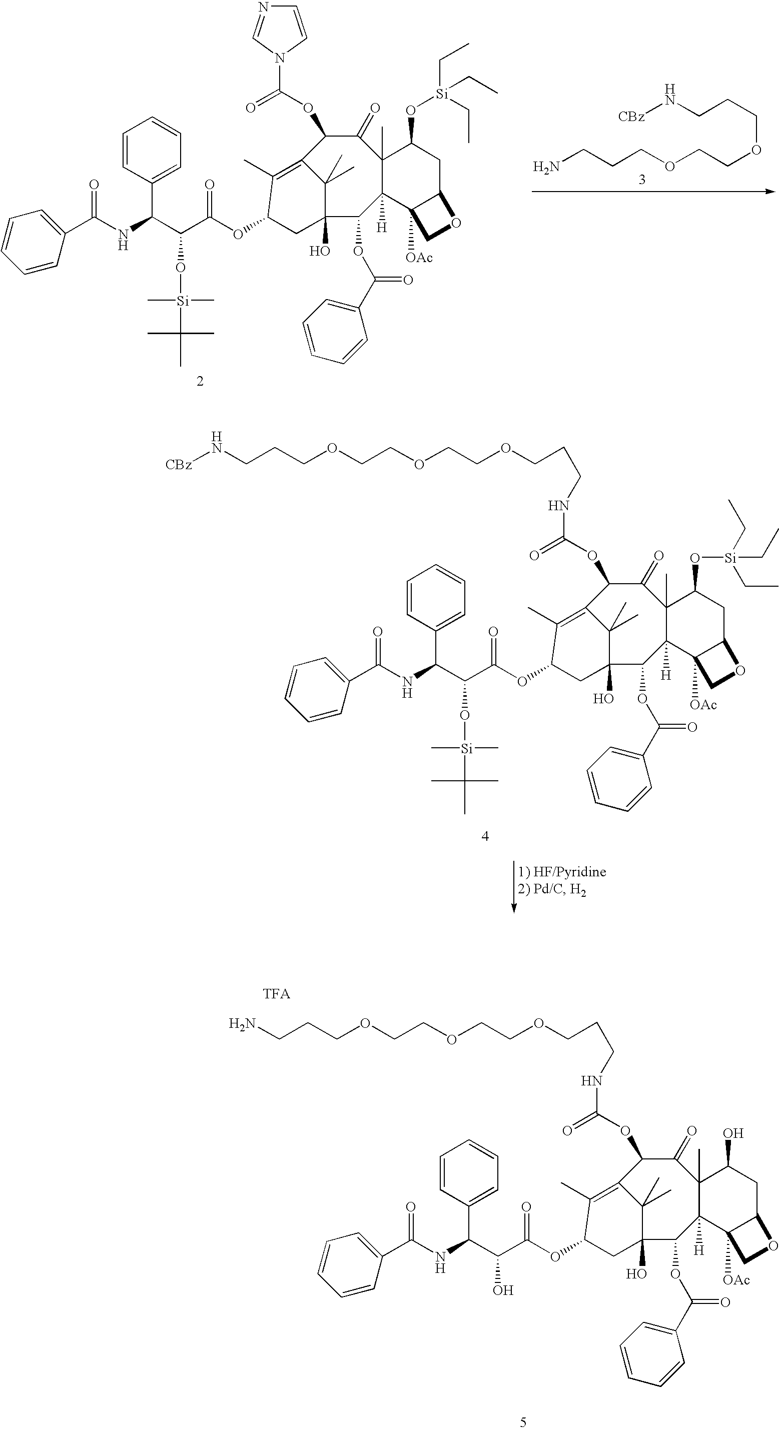

### Example 4

### Preparation of -10-O-(deacetyl)-10-O-(N-(4-(3-carboxylic acid)-propyl)-phenyl)-aminocarbonyl)-paclitaxel (6)

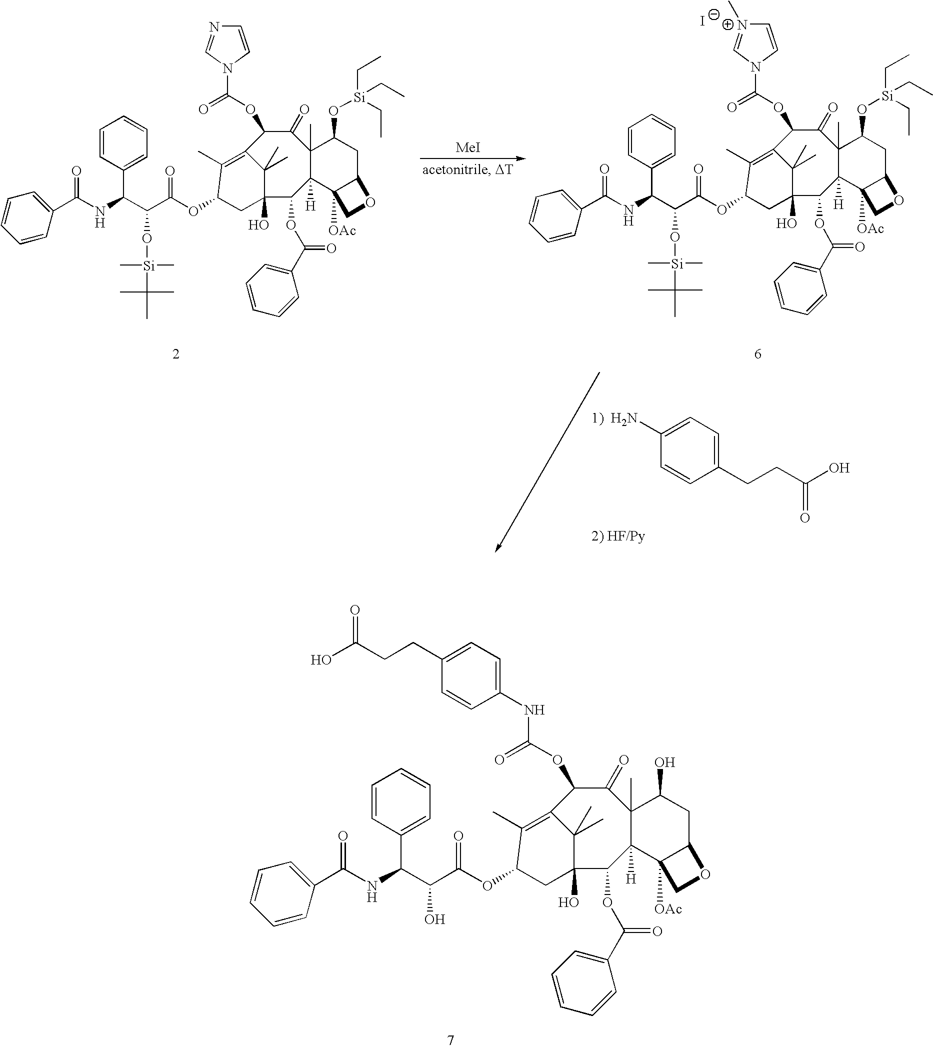

To (20 mg, 0.018 mmol) 2′-O-(tert-butyldimethylsilyl)-7-O-(triethylsilyl)-10-deacetyl-10-O-(imidazoylcarbonyl)-paclitaxel (2), prepared according to the procedure of Example 1, dissolved in CH₃CN (0.3 mL), is added MeI (0.2 mL). The reaction mixture is stirred for 3 hours at 55° C. in a sealed tube. A stream of N₂ is then used to remove the volatiles and the residue is exposed to high-vacuum to remove volatiles giving intermediate 6. The imidazolium salt 6 is then dissolved in DMSO (0.5 mL) and 3-(4-amino-phenyl)-propionic acid (500 mol %) is added. The reaction mixture is stirred for 30 minutes at room temperature, diluted with pyridine (0.5 mL). The resulting mixture is cooled to 0° C. and HF/Py (233 μl) is added. Stirring is continued for 3 hours at room temperature. The reaction mixture is then diluted with EtOAc (5 mL) and extracted with saturated aqueous solution of CuSO₄ (3×1 mL) followed by water (2×2 mL). The organic phase is then dried over sodium sulfate, filtered and concentrated. The resulting residue is purified by preparative RP-HPLC C-18 column (Method A). Fractions containing the appropriate mass, as determined by analytical HPLC-MS (Method B) are pooled and CH₃CN is removed under reduced pressure. The remaining aqueous mixture is then lyophilized to give the desired paclitaxel-linker construct 7 with aryl carbamate linker at C10.

### Example 4a

### Preparation of 10-O-deacetyl-10-O-(N-(2-deoxyglucosyl)-aminocarbonyl)-paclitaxel (7a)

To the imidazolium salt 6 prepared according to the procedure of Example 4, dissolved in DMSO (0.5 mL), was added D-glucosamine hydrochloride (500 mol %) followed by DIEA (500 mol %). The reaction mixture was stirred for 30 minutes at 55° C. and then diluted with pyridine (0.5 mL). After cooling to 0° C., HF/Py (233 ml) was added and the resulting mixture was stirred for an additional 3 hours at room temperature. The reaction mixture was then diluted with EtOAc (5 mL) and extracted with saturated aqueous solution of CuSO4 (3×1 mL) followed by water (2×2 mL). The organic phase was then dried over sodium sulfate, filtered and concentrated. The resulting residue was purified by preparative RP-HPLC C-18 column (Method A). Fractions containing the appropriate mass, as determined by analytical HPLC-MS (Method B) were pooled and CH3CN was removed under reduced pressure. The remaining aqueous mixture was then lyophilized to give paclitaxel-sugar-conjugate of formula 7a. Electrospray (LCMS) m/z 1017 (M+H+, C52H61N2O19 requires 1017); retention time 5.12 min. (1%-99% B, Method B)

### Example 5

### Preparation of mono-N-Boc-2-[2-[2-[2-aminoethoxy]ethoxy]ethoxy]ethylaminediaminoPEG (9)

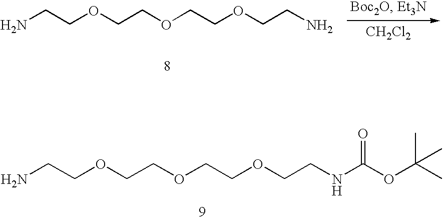

To 2-[2-[2-[2-aminoethoxy]ethoxy]ethoxy]ethylaminediaminoPEG (8 (, 0.5 g, 2.6 mmol), dissolved in CH₂Cl₂ (50 mL), was added triethylamine (0.36 mL, 100 mol %) and Boc₂O (0.55 g, 100 mol %). The reaction mixture was stirred for 4 hours and concentrated to dryness. The resulting residue was purified by silica gel column chromatography eluting with 9:1:0.1 chloroform:methanol:ammonium hydroxyde to give 0.26 g (34% yield) of the title compound 9. ¹H NMR (CDCl₃, 300 MHz) δ 3.66 (m, 8H), 3.57 (m, 4H), 3.28 (m, 2H), 2.90 (t, 2H), 1.63 (bs, 2H), 1.47 (s, 9H).

### Example 6

### Preparation of deacetylvinblastine-3-(amido-PEG-amine) Reaction Between 4-deacetyl-3-de-(methoxycarbonyl)-vinblastin-3-yl-carbonyl Azide (10) and N-Boc-2-[2-[2-[2-aminoethoxy]ethoxy]ethoxy]ethylamine (9)

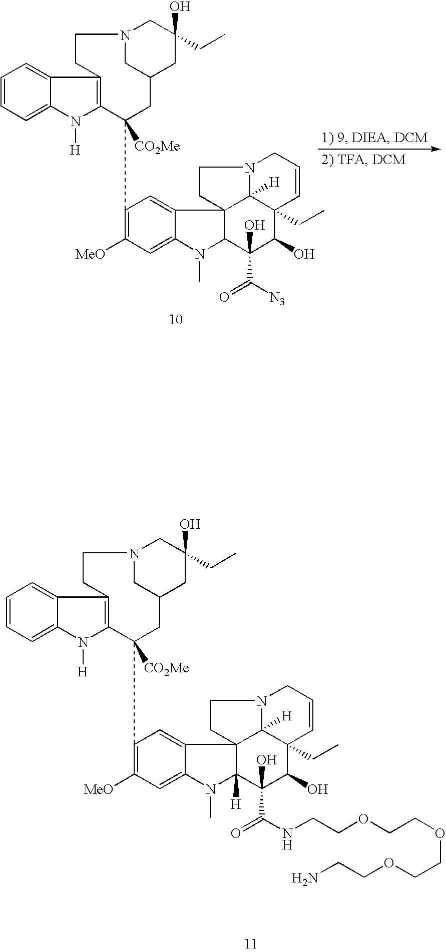

To a DCM solution of 4-deacetyl-3-de-(methoxycarbonyl)-vinblastin-3-yl-carbonyl azide (10deacetylvinblastine acid azide, 0.46 mmol)), prepared according to the procedure of K. S. P. Bhushana Rao et al., J. Med. Chem. (1985), 28: 1079, was added N-Boc-2-[2-[2-[2-aminoethoxy]ethoxy]ethoxy]ethylamine (the mono Boc-protected diamine 9, (0.2 g, 150 mol %), followed by DIEA (0.12 mL, 150 mol %). The reaction mixture was stirred at room temperature for 3 hours then concentrated in vacuo to give a residue that was purified by silica gel column chromatography eluting with 95:5 chloroform:methanol. The intermediate, 4-deacetyl-3-de-(methoxycarbonyl)-vinblastin-3-yl-N-{N′-Boc-2-[2-[2-[2-ethoxy]ethoxy]ethoxy]ethylamino}-aminocarbonyl) was dissolved into 120 mL 1:1 of DCM:TFA and the mixture was stirred at room temperature for 10 minutes. The mixture was concentrated with a flow of N₂ and the resulting residue lyophilized to give 0.31 g (65% overall yield) of the desired vinblastine-linker construct 11 with PEG amide linker at C3 which was used without further purification. Electrospray (LCMS) m/z 929.5 (M+H⁺, C₅₁H₇₃N₆O₁₀ requires 929.5); retention time 3.46 min. (1% to 99% B, Method B).

### Example 7

### Preparation of (6-hydroxy-hexyl)-carbamic Acid Benzyl Ester (13)

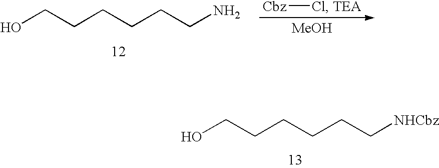

To 6-aminohexan-1-ol (12, 0.49 g, 100 mol %) in MeOH (25 mL) were added benzylchloroformate (1.0 mL, 165 mol %) and triethylamine (1.0 mL, 165 mol %). The reaction mixture was stirred for 5 hours at room temperature then concentrated to dryness to give a residue which was purified by silica gel column chromatography using 1:1 hexanes:EtOAc to give 0.9 g (85% yield) of title compound 13. ¹H NMR (CDCl₃, 300 MHz) δ 7.37 (m, 5H), 5.13 (s, 2H), 3.67 (m, 2H), 3.24 (m, 2H), 1.56 (m, 4H), 1.35 (m, 2H)

### Example 8

### Preparation of (6-Bromo-hexyl)-carbamic Acid Benzyl Ester (14)

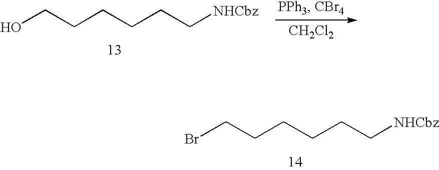

To 13 (0.53 g, 100 mol %) dissolved in DCM (50 mL) were added triphenylphosphine (0.66 g, 120 mol %) and carbon tetrabromide (0.84 g, 120 mol %). The reaction mixture was stirred for 90 minutes at room temperature then concentrated to dryness to give a residue which was purified by silica gel column chromatography eluting with 1:1 hexanes:EtOAc to give 0.32 g (48%) of title compound 14. ¹H NMR (CDCl₃, 300 MHz) δ 7.39 (m, 5H), 5.13 (s, 2H), 3.43 (t, 2H), 3.25 (m, 2H), 1.74 (m, 2H), 1.45 (m, 6H)

### Example 9

### Reaction of Thymidine with (6-Bromo-hexyl)-carbamic Acid Benzyl Ester (14) and Deprotection to Give 3-(6″-aminohexyl)-thymidine (15)

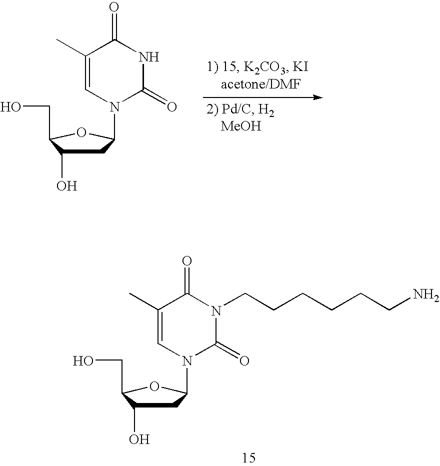

To thymidine (0.2 g, 100 0.83 mmol %) dissolved in acetone (3 mL) and DMF (3 mL) were added 14 (0.26 g, 100 mol %) and K₂CO₃ (0.22 g, 200 mol %). The reaction mixture was stirred at 50° C. for 48 hours then partitioned between EtOAc and water. The aqueous layer was extracted with ethyl acetate and the organic layer was dried over Na₂SO₄ and concentrated to dryness to give a residue which was purified by silica gel column chromatography eluting with 95:5 chloroform:methanol. The CBz protected thymidine-N³-linker intermediate so obtained was dissolved in methanol (10 mL) and 10 wt % palladium on carbon (33 mg) was added. The reaction mixture was stirred at room temperature under 1 atm of H₂ for 16 hours then filtered through Celite. The filtrate was concentrated under reduced pressure to give 0.17 g (60% overall yield) of the title compound 15. ¹H NMR (CDCl₃, 300 MHz) δ 7.88 (s, 1H), 6.33 (t, J=7 Hz, 1H), 3.96 (m, 3H), 3.78 (m, 2H), 2.96 (m, 2H), 2.54 (m, 2H), 1.94 (s, 3H), 1.65 (m, 4H), 1.45 (m, 4H)

### Example 10

### Preparation of 2′-O-(benzyloxycarbonyl)-7-O-(4-nitrophenyloxycarbonyl)-paclitaxel (17)

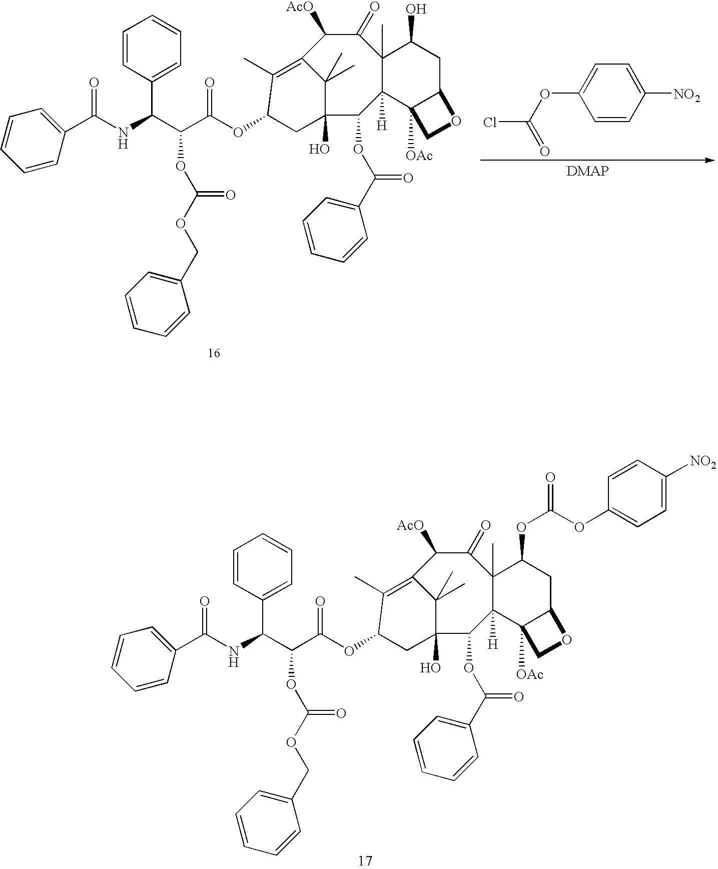

To 2′-O-(benzyloxycarbonyl)-paclitaxel (16, 0.52 g, 0.53 mmol), prepared according to the procedure described in. Chen, S.-H., et al., Tetrahedron (1993) 49: 2805-2828, dissolved in DCM (150 mL) were added p-nitrophenylchloroformate (0.64 g, 600 mol %) and DMAP (0.6 g, 920 mol %). The reaction mixture was stirred for 2 hours and concentrated to dryness. The resulting residue was purified by silica gel column chromatography eluting with 1:1 hexanes:EtOAc to give 0.48 g (79% yield) of the title compound 17: ¹H NMR (CDCl₃, 300 MHz) δ 8.31 (d, J=9 Hz, 2H), 8.17 (d, J=9 Hz, 2H), 7.77 (d, J=8 Hz, 2H), 7.32 (m, 18H), 6.98 (d, J=9 Hz, 1H), 6.43 (s, 1H), 6.31 (t, J=9 Hz, 1H), 6.01 (d, J=9 Hz, 1H), 5.18 (s, 2H), 5.03 (d, J=9 Hz, 1H), 4.40 (d, J=8 Hz, 1H), 4.25 (d, J=8 Hz, 1H), 4.03 (d, J=8 Hz, 1H), 2.77 (m, 2H), 2.50 (s, 3H), 2.33 (m, 2H), 2.24 (s, 3H), 2.08 (m, 4H), 1.91 (s, 3H), 1.80 (s, 3H), 1.28 (m, 6H); Electrospray (LCMS) m/z 1154 (M+H⁺, C₆₂H₆₁N₂O₂₀ requires 1154); retention time=8.48 min (1% to 99% B, Method B).

### Example 11

### Reaction of 2′-O-(benzyloxycarbonyl)-7-O-(4-nitrophenyloxycarbonyl)-paclitaxel7-(p-nitrophenylcarbonyl)paclitaxel with 3-(6″-aminohexyl)thymidine and Deprotection to Give of paclitaxel-7-O-(N-(6-hexyl-[thymidin-3-yle)]hexan-1-yl) aminocarbonyl)-paclitaxel (18)

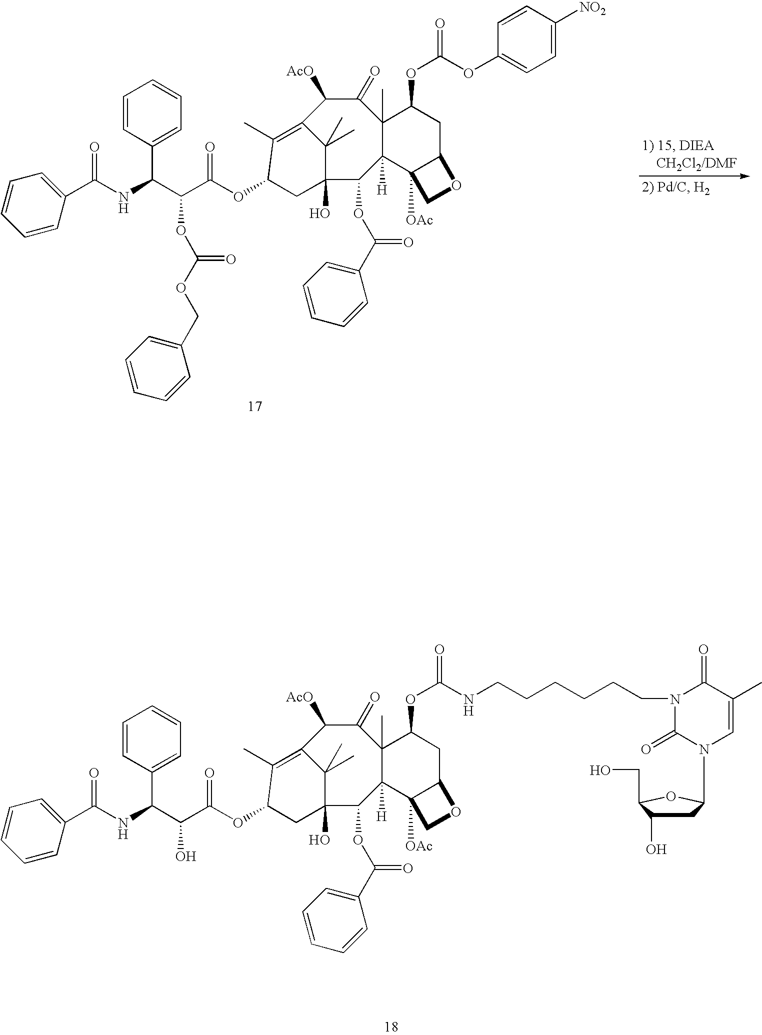

To 2′-O-(benzyloxycarbonyl)-, 7-O-(p-nitrophenyloxycarbonyl)-paclitaxel (17, 50 mg, 0.043100 mmol %), dissolved in DMF (2 mL) and CH₂Cl₂ (3 mL), was added 3-(6″-aminohexyl)-thymidine (15, 57 mg, 380 mol %) followed by DIEA (30 μL, 380 mol %). The reaction mixture was stirred at room temperature for 3 hours then partitioned between EtOAc and water. The aqueous layer was extracted with EtOAc and the organic layer was dried over Na₂SO₄ and concentrated to dryness to give a residue directly injected onto a preparative RP-HPLC C-18 reversed phase column for purification (Method A). Fractions containing the appropriate mass, as determined by analytical HPLC-MS (Method B), were pooled and the solvent was removed under reduced pressure. To the paclitaxel-thymidine intermediate so obtained, dissolved in methanol (5 mL), was added 10 wt % palladium on carbon (13 mg) and the reaction mixture was stirred at room temperature under 1 atm of H₂ for 16 hours. The mixture was filtered through Celite and concentrated under reduced pressure to give 37 mg (71% yield) of the title compound 18. Electrospray (LCMS) m/z 1221 (M+H⁺, C₆₄H₇₇N₄O₂₀ requires 1221); retention time=6.20 min (1% to 99% B, Method B).

### Example 12

### Preparation of 3-(2,5-dioxo-2,5-dihydropyrrol-1-yl)propionaldehyde (19)

To 1-(3-hydroxypropyl)-1H-pyrrole-2,5-dione (200 mg, 1.29 mmol) dissolved in 5 mL DCM. DMP (15% wt in DCM, 4 mL, 1.93 mmol) was added in one portion. After stirring the mixture for 2 h, 2-propanol (3 mL) was added followed by stirring for an additional 30 min. The resulting solution was filtered through a silica gel pad eluted with EtOAc, and the filtrate was concentrated. The crude product was purified by silica gel chromatography eluting with EtOAc/Hexane (2/1) to provide 3-(2,5-Dioxo-2,5-dihydro-pyrrol-1-yl)-propionaldehyde (110.0 mg, 0.72 mmol, 56% yield) which was used immediately. ¹H NMR (CDCl₃, 300 MHz) δ 9.74(t, 1H, J=1.2 Hz), 6.69 (s, 2H), 3.84 (t, 2H, J=6.9 Hz), 2.77 (td, 2H, J=6.9, 1.2 Hz).

### Example 13

### Preparation of N3′-maleimidopropyl Doxorubicin (20)

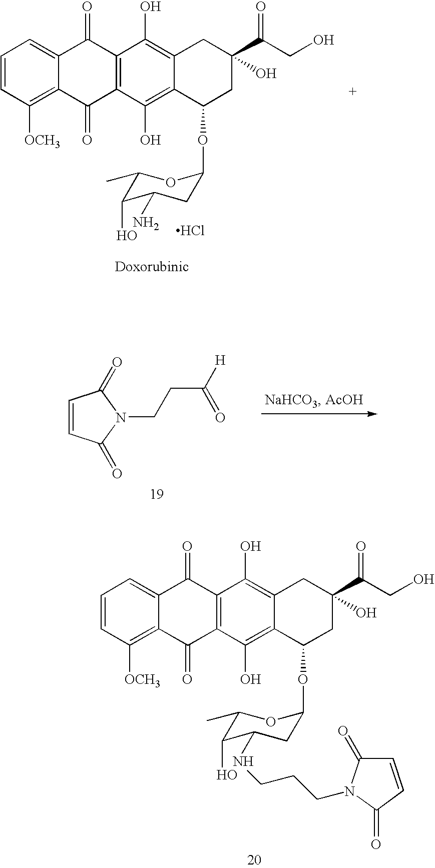

To a stirred solution of doxorubicin hydrochloride (100 mg, 0.172 mmol), 19 (68.2 mg, 0.446 mmol) and glacial AcOH (20 μL, 195 mol %) in CH₃CN/H₂O (2:1, 5 mL) was added a 1 M solution of NaCNBH₃ in THF (57 μL, 0.33 mol %). The mixture was stirred under nitrogen atmosphere in the dark at RT for 1 h. The solution was then concentrated in vacuo to give a residue which was diluted with an aqueous 5% NaHCO₃ solution and extracted with DCM. Concentration of the organic solution and purification of the resulting residue by silica gel chromatography eluting with DCM/CH₃OH (20:1) provided 26.0 mg of N-3-maleimidopropyl doxorubicin 20 (21.4% yield). ¹H NMR (CDCl₃, 300 MHz) δ 8.03 (d, 1H, J=8.4 Hz), 7.79 (t, 1H, J=8.4 Hz), 7.41 (d, 1H, J=8.4), 6.68 (s, 2H), 5.51 (m, 1H), 5.32 (m, 1H), 4.82-4.76 (m, 2H), 4.09 (s, 3H), 3.96 (m, 1H), 3.58 (m, 3H), 3.32-2.98 (m, 2H), 2.76 (m, 1H), 2.54 (m, 2H), 2.37 (m, 1H), 2.15 (m, 1H), 1.85-1.54 (m, 4H), 1.37 (d, 3H, J=7.0 Hz). Electrospray (LCMS) m/z 681.2 (M+H⁺, C₃₄H₃₆N₂O₁₃ requires 681.2)

### Example 14

### Preparation of (6-(thymidin-3-yl)-hexan-1-yl) 3-mercaptopropanamide (21)

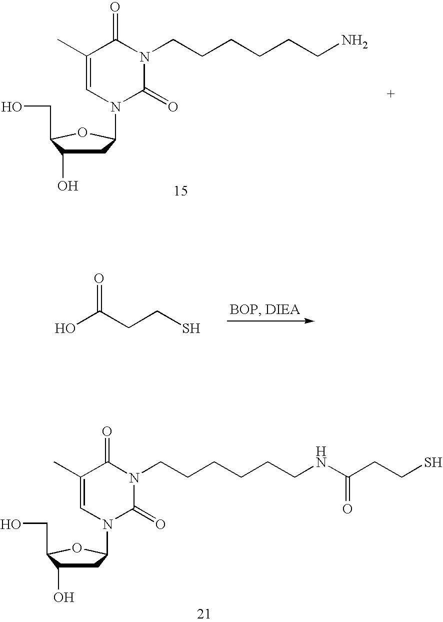

To a solution of 3-(6-aminohexyl) thymidine (15, 81.0 mg, 0.237 mmol) in DMF prepared according to the procedure described herein were added BOP (192 mg, 0.350 mmol), DIEA (123 mg, 0.948 mmol) and 3-mercaptopropionic acid (37.2 mg, 0.350 mmol). The reaction mixture was stirred for 30 min whereupon DMF was removed in vacuo. The crude was purified by silica gel P-TLC eluting with DCM/CH₃OH (9:1) to give 51.3 mg compound 23 (50.4% yield). ¹H NMR (CDCl₃, 300 MHz) δ 7.40 (s, 1H), 6.16 (t, 1H, J=6.6 Hz), 6.0 (m, 1H), 4.60 (m, 1H), 4.06-3.78 (m, 4H), 3.70 (m, 1H), 3.29-3.11 (m, 3H), 2.81 (q, 2H, J=7.5 Hz), 2.50 (t, 2H, J=6.6 Hz), 2.46-2.27 (m, 2H), 1.92 (s, 3H), 1.68-1.17 (m, 8H). Electrospray (LCMS) m/z 430.2 (M+H⁺, C₁₉H₃₁N₃O₆S requires 430.2)

### Example 15

### Preparation of 3-(1-(Doxorubicin-N^{3′}-propyl)-2,5-dioxopyrrolidin-3-ylthio)-N-(6-(thymidin-3-yl)-hexylpropanamide (22)

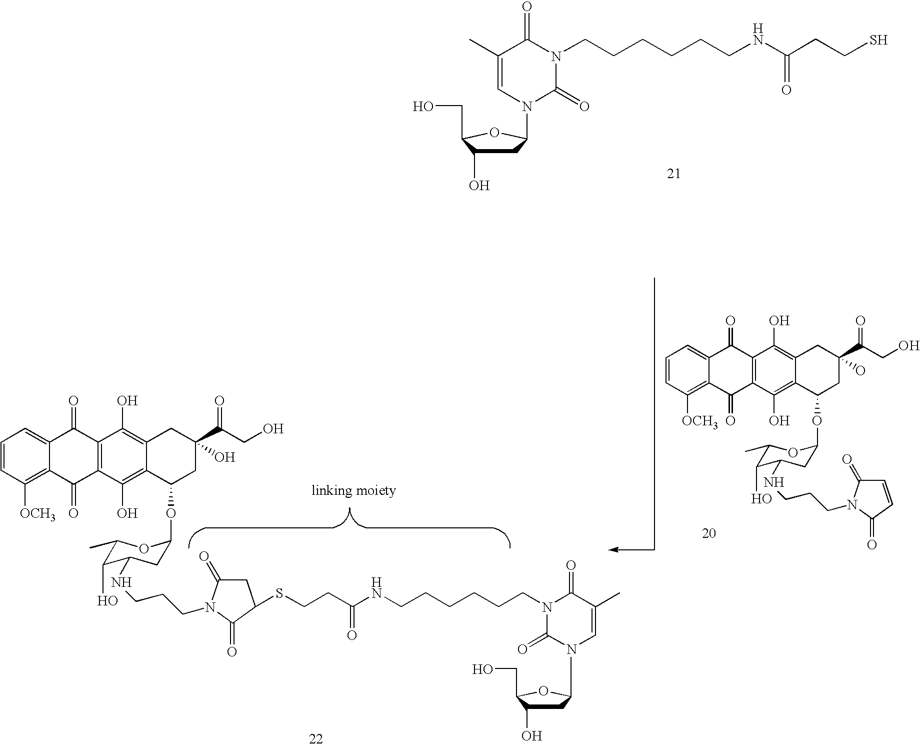

To a DCM/CH₃OH (9:1) solution of N-3-maleimidopropyl doxorubicin 20 (17.5 mg, 0.026 mmol) was added the thiol containing thymidine derivative 21 (11.2 mg, 0.026 mmol). The mixture was stirred under nitrogen atmosphere in the dark for 30 min. The solvent was removed in vacuo and the resulting crude residue was dissolved into by DMSO and purified on a preparative RP-HPLC C-18 reversed phase column (Method A). Fractions containing the appropriate mass, as determined by analytical HPLC-MS (Method B), were pooled and CH₃CN was removed under reduced pressure or N₂ stream followed by lyophilization to give 6.1 mg of the anthracycline-linker-thymidine conjugate 22 (21% yield). Electrospray (LCMS) m/z 1110.5 (M+H+, C₅₃H₆₇N₅O₁₉S requires 1110.4)

### Example 16

### Cytotoxicity Assay

Cytotoxicity Assay (Monolayer)

Monolayer assays with tumor cell lines (MCF-7 breast carcinoma and HT-29 colorectal carcinoma from ATCC) were carried out in triplicate in 96-well plates with RPMI1640 medium containing 5% fetal bovine serum, 100 U/ml penicillin and 100 μg/ml streptomycin. Normal human foreskin fibroblasts (HFF #CC-2509) were from Cambrex and were cultured in FGM-2 medium. Exponentially growing cells (5,000 MCF-7 or HT-29; 1,500 HFF) were plated in 100 μl medium and incubated overnight (5% CO₂, 37° C.). Compounds (20 pM to 20 μM final concentration, 6-8 doses) and vehicle (DMSO) controls were added and the incubation was continued for an additional 72 hours. Final cell density was determined by incubating cultures with 25 μl AlamarBlue reagent (BioSource, Camarillo, Calif.) for 4 hours, followed by determination of fluorescence at excitation of 544 nm and emission of 590 nm with a SpectroMax Gemini EM fluorescence plate reader (Molecular Devices, Sunnyvale, Calif.). EC₅₀ values were generated from dose-response curves by a 4-parameter method using Softmax PRO software. Mean EC₅₀s (±SD) represent the average of all tests carried out for all lots of a given compound. Outlier EC₅₀ values (<7%) were identified and removed prior to analysis using the method of Hoaglin et al., J. Amer. Statistical Assoc., 81, 991-999 (1986).

Cytotoxicity Assay (Soft Agar)

Assays were carried out in 24-well plates with 0.5 ml bottom layers (0.8% agar) and 0.5 ml top layers (0.38% agar) in RPMI1640 medium containing 5% fetal calf serum. Top layers were plated with 1,250 MCF-7 or 5,000 HT-29 cells per well and drugs, compounds or vehicle controls in triplicate as described above. Plates were incubated as above for 10-14 days and then colony formation was assessed by adding 50 μl AlamarBlue to each well and determining EC₅₀s as described above for monolayer assays. The EC₅₀ values for exemplary conjugates and patent drugs for normal and tumor cells different cell lines are provided in FIG. 1-3.

### Example 17

### Thymidine Kinase Cloning, Expression, Assay

Human thymidine kinase 1 (TK1) cDNA clone (cat. # OHS1166-7304119) was obtained from Open Biosystems (Huntsville, Ala.). TK1 was amplified using PCR with forward primer 5′-CAATCCATATGAGCTGCATTAACCTGC-3′ and reverse primer 5′-TATTAAGCTTCTAGTTGGCAGGGCTGCAT-3′. The PCR product was digested with Nde I and Hind III. An N-terminal His-tagged TK1 construct, TK1/pET28b(+), was generated by subcloning TK1 into the Nde I and Hind III sites of prokaryotic expression vector pET28b (+) (Novagen, San Diego, Calif.). TK1 was expressed from TK1/pET28b(+) using E. coli strain BL21-codon plus (Stratagene, San Diego, Calif.) and purified by Ni column chromatography.

A coupled (kinetic) ATP depletion assay was developed to measure thymidine kinase activity. The reaction contained 100 mM Tris HCl, pH 7.5, 20 mM MgCl₂, 100 mM KCl, 0.4 mM PEP, 0.2 mM NADH, Pyruvate Kinase (0.7 units)/Lactate Dehydrogenase (1.0 unit) (Sigma #PO₂₉₄), 2.5-10 μg Thymidine Kinase, 100 μM thymidine or thymidine-drug conjugate in a volume of 75 μl. ATP (5 mM in 25 μl H₂O) was added to initiate the reaction and the velocity of ATP depletion was monitored at 340 nm continuously for 15 minutes. Results for conjugate phosphorylation represent the initial velocity relative to thymidine and are provided in Tables 7-9 for exemplary conjugates.

### Example 18

### Fluorescence-Based Assays for Enhancement (Paclitaxel) and Inhibition (Vinblastine) of Microtubule Polymerization

The assay kit (#BK011) was purchased from Cytoskeleton (Denver, Colo.). The assays were carried out according to the manufacturer's instructions, except that 1 mg/ml BSA (Sigma #A3059) was included in all assays. Paclitaxel assays were carried out in the absence of glycerol and vinblastine assays were carried out in the presence of 20% glycerol. Parent drugs and conjugates were tested at 0.75, 1.5, 3 and 10 micromolar final concentration and results represent a comparison of conjugate and parent drug curves obtained from the linear range of the dose responses. Mean percentages of paclitaxel or vinblastine activity (±SD) represent the average of all tests carried out for all lots of a given compound. Results for exemplary conjugates are provided in Tables 7-9.

### Example 19

### Topoisomerase II Assay

Doxorubicin conjugates were assayed for their effect on Topoisomerase II using the Topoisomerase II Drug Screening Kit (Catalog # 1009-1) produced by TopoGEN Inc. (Columbus, Ohio). Specifically the kit was used to assay whether Doxorubicin conjugates altered the ability of Topoisomerase II to catalyze the formation of relaxed conformation DNA from a super-coiled plasmid. Doxorubicin conjugates were compared directly to Doxorubicin at 10, 3, 1, 0.3, 0.1 and 0.03 micromolar concentrations. The quantity of relaxed conformation DNA was quantified from an agarose gel on which is it is separated from the super-coiled DNA by standard electrophoresis. The more active a drug is at a particular concentration the less relaxed conformation DNA is produced by the action of Topoisomerase II. The results are presented in terms of percent activity of Doxorubicin. Results for exemplary conjugates are provided in Tables 7-9.

### Example 20

### Serum Stability

The stability of conjugates was measured in RPMI1640 cell culture medium containing 10% fetal bovine serum. The serum-containing medium was pre-warmed at 37° C. for 3 min prior to addition of test articles. Test articles, prepared in DMSO as 5 mM stocks, were added to the cell culture media to a final concentration of 10 mM. Aliquots (150 ml) were withdrawn in triplicate at 0, 4, 8, 24 and 72 hours and combined with the same volume of ice-cold acetonitrile to terminate the reaction. The mixture was centrifuged at 2,000×g for 10 minutes. One part of the supernatant was mixed with four parts of deionized water to bring down the percentage of organic solvent. The diluted samples were then assayed by LC/MS for the test article. The natural log of the percent remaining was plotted versus time. A linear fit was used to determine the rate constant. The fit was truncated after the percent of remaining test article was less than 10%. The elimination half-lives associated with the disappearance of test articles were determined to compare their relative stability. The assays were carried out by Absorption Systems (Exton, Pa.).

### Example 21

### Liver Microsome Metabolic Stability

Human and mouse liver microsomes were obtained from Absorption Systems (Exton, Pa.) and Xenotech (Lenexa, Kans.), respectively. The reaction mixture contained microsomes (human or mouse) 1.0 mg/ml, potassium phosphate, pH 7.4 100 mM, magnesium chloride 10 mM, test article 10 mM, and was equilibrated at 37° C. for 3 min. The reaction was initiated by adding NADPH (1 mM final), and the system was then incubated in a shaking water bath at 37° C. Aliquots (100 ml) were withdrawn in triplicate at 0, 15, 30, and 60 minutes and combined with 900 ml of ice-cold 50/50 acetonitrile/dH2O to terminate the reaction. Two controls (testosterone and propranolol) were run simultaneously with the test articles in separate reactions. The samples were assayed by LC/MS for the test article. The natural log of the percent remaining was plotted versus time. A linear fit was used to determine the rate constant. The fit was truncated when percent remaining of the test article was less than 10%. The elimination half-lives associated with the disappearance of test and control articles were determined to compare their relative metabolic stability. The assays were carried out at Absorption Systems (Exton, Pa.).

### Example 22

### Thymidine Kinase-Mediated Drug Trapping Results

The pharmacological activity of a paclitaxel-thymidine conjugate and a vinblastine-thymidine conjugate were compared to the corresponding unconjugated paclitaxel and vinblastine. The paclitaxel-thymidine conjugate shows a TK substrate activity corresponding to 25% of thymidine, and a paclitaxel activity corresponding to 77% of paclitaxel. The paclitaxel-thymidine conjugate exhibits cytotoxity against breast carcinoma, colon carcinoma and leukemia. EC₅₀ values for paclitaxel and paclitaxel-thymidine conjugate were determined to be 5-8 nM and 75-170 nM, respectively.

The vinblastine-thymidine conjugate shows a TK substrate activity corresponding to 27% of thymidine, and a vinblastine activity corresponding to 76% of vinblastine. The vinblastine-thymidine conjugate exhibits cytotoxicity against breast carcinoma, colon carcinoma and leukemia. EC₅₀ values for vinblastine and vinblastine-thymidine conjugate were determined to be 1-2 nM and 11-43 nM, respectively.

### Example 23

A comparison of cytotoxic selectivity index for an exemplary conjugate and parent drug in tumors and normal cells shows the increase in the cytotoxic selectivity index of the conjugate for tumor cells as compared to the cytotoxic selectivity index of the parent drug:

| | HFF | | | | |
|---|---|---|---|---|---|
| | Monolayer | MCF-7 | MCF-7 Soft | HT-29 | HT-29 Soft |
| | EC50 | Monolayer | Agar | Monolayer | Agar |
| | (nM) | EC50 (nM) | EC50 (nM) | EC50 (nM) | EC50 (nM) |
| Paclitaxel | 9 ± 5 | 5 ± 2 | 6 ± 3 | 5 ± 3 | 15 ± 2 |
| | (n = 20) | (n = 19) | (n = 8) | (n = 18) | (n = 6) |
| PXL-7Ca-ALK(6)-N3-Thy | 457 ± 310 | 120 ± 73 | 40 ± 41 | 258 ± 129 | 120 ± 4 |
| | (n = 16) | (n = 16) | (n = 9) | (n = 16) | (n = 5) |

1. A conjugate, comprising a drug and a substrate for a protein kinase or a lipid kinase non-releasably linked thereto, optionally via a non-releasable linker.

2. The conjugate of claim 1, wherein a significant fraction of a biological activity of the drug is retained in the conjugate.

3. The conjugate of claim 1, wherein more than 50% of the biological activity is retained in the conjugate.

4. The conjugate of claim 1, wherein more than 20% of the biological activity is retained in the conjugate.

5. The conjugate of claim 1, wherein more than 5% of the biological activity is retained in the conjugate.

6. The conjugate of claim 1 that comprises:
(substrate)ₜ, (Linker)_{q}, and (drug)_{d};
wherein at least one substrate moiety is linked, optinally via a non-releasable linker to at least one drug, t is 1 to 6, q is 0 to 6, and d is 1 to 6.

7. The conjugate of claim 1, wherein the kinase is overexpressed, overactive or that exhibits undesired activity in a target system.

8. The conjugate of claim 1, wherein the kinase is associated with an ACAMPS-related condition.

9. The conjugate of claim 1, wherein the substrate is a substrate for a nucleoside kinase.

10. The conjugate of claim 1, wherein the substrate is a substrate for a thymidine kinase, deoxycytidine kinase or deoxyguanosine kinase.

11. The conjugate of claim 1, wherein the substrate is a substrate for viral thymidine kinase or human thymidine kinase.

12. The conjugate of claim 1, wherein the substrate is a natural or a non-natural nucleoside.

13. The conjugate of claim 1, wherein the substrate is a natural or a non-natural nucleoside that is converted to a substrate of thymidine kinase or deoxycytidine kinase by an action of thymidine phosphorylase or cytidine deaminase.

14. The conjugate of claim 9, wherein the nucleoside is a pyrimidine or a purine nucleoside, or a pharmaceutically acceptable derivative thereof.

15. The conjugate of claim 9, wherein the nucleoside is a pyrimidine nucleoside or a pharmaceutically acceptable derivative thereof.

16. The conjugate of claim 9, wherein the nucleoside is a purine nucleoside or a pharmaceutically acceptable derivative thereof.

17. The conjugate of claim 9, wherein the nucleoside is a pyrimidine covalently linked to a deoxyribose sugar.

18. The conjugate of claim 9, wherein the nucleoside is a pyrimidine covalently linked to a ribose sugar.

19. The conjugate of claim 9, wherein the nucleoside comprises a purine covalently linked to a deoxyribose sugar.

20. The conjugate of claim 9, wherein the nucleoside comprises a purine covalently linked to a ribose sugar.

21. The conjugate of claim 9, wherein the nucleoside comprises a base selected from cytosine, uridine, thymidine, guanosine, adenosine, or a pharmaceutically acceptable derivative thereof.

22. The conjugate of claim 9, wherein the nucleoside is thymidine.

23. The conjugate of claim 1, wherein the substrate is a nucleoside or nucleoside analog substrate for a thymidine kinase.

24. The conjugate of claim 1, wherein the substrate is a nucleoside or nucleoside analog substrate for human thymidine TK-1 or a viral TK.

25. The conjugate of claim 1, wherein the drug is a cytotoxic agent.

26. The conjugate of claim 1, wherein the drug is a label.

27. The conjugate of claim 1, wherein the drug is an anti-infective agent, antihelminthic agent, antiprotozoal agent, antimalarial agent, antiamebic agent, antileiscmanial agent, antitrichomonal agent, antitrypanosomal agent, sulfonamide, antimycobacterial agent, or antiviral agent.

28. The conjugate of claim 1, wherein the drug is an alkylating agent, plant alkaloid, antimetabolite, antibiotic, microtubue or tubulin binding agent.

29. The conjugate of claim 1, wherein the drug is a central nervous system depressant and stimulant, respiratory tract drug, pharmacodynamic agent, cardiovascular agent, blood or hemopoietic system agent, gastrointestinal tract agent, or locally acting chemotherapeutic agent.

30. The conjugate of claim 1, wherein the drug is selected from among the following classes of drugs:
a) anthracycline family of drugs,
b) vinca alkaloid drugs,
c) mitomycins,
d) bleomycins,
e) cytotoxic nucleosides,
f) pteridine family of drugs,
g) diynenes,
h) estramustine,
i) cyclophosphamide,
j) taxanes,
k) podophyllotoxins,
l) maytansanoids,
m) epothilones, and
n) combretastatin and analogs,
or pharmaceutically acceptable derivatives thereof.

31. The conjugate of claim 1, wherein the drug is selected from among the following drugs:
a) doxorubicin,
b) carminomycin,
c) daunorubicin,
d) aminopterin,
e) methotrexate,
f) methopterin,
g) dichloromethotrexate,
h) mitomycin C,
i) porfiromycin,
j) 5-fluorouracil,
k) 6-mercaptopurine,
l) cytosine arabinoside,
m) podophyllotoxin,
n) etoposide,
o) etoposide phosphate,
p) melphalan,
q) vinblastine, r) vincristine,
s) leurosidine,
t) vindesine,
u) estramustine,
v) cisplatin,
w) cyclophosphamide,
x) paclitaxel,
y) leurositte,
z) 4-desacetylvinblastine,
aa) epothilone B,
bb) docetaxel,
cc) maytansanol,
dd) epothilone A, and
ee) combretastatin and analogs;
or a pharmaceutically acceptable derivative thereof.

32. The conjugate of claim 1 comprising a non-releasable linker.

33. The conjugate of claim 1, wherein the linker comprises linear or acyclic portions, cyclic portions, aromatic rings or combinations thereof.

34. The conjugate of claim 1, wherein the linker comprises linear or acyclic portions.

35. The conjugate of claim 1, wherein the linker comprises up to 50 main chain atoms.

36. The conjugate of claim 1, wherein the linker comprises up to 30 main chain atoms.

37. The conjugate of claim 1, wherein the linker comprises up to 20 main chain atoms.

38. The conjugate of claim 1, wherein the linker comprises up to 10 main chain atoms.

39. The conjugate of claim 1, wherein the linker comprises up to 5 main chain atoms.

40. The conjugate of claim 1, wherein the linker comprises oligomers of ethylene glycol or straight alkelene chains or mixtures thereof.

41. The conjugate of claim 1, wherein the linker comprises polyethylene glycol.

42. The conjugate of claim 41, wherein the polyethylene glycol comprises 5, 11, 13, 14, 22 or 29 atoms in the chain.

43. The conjugate of claim 41, wherein the polyethylene glycol comprises 5, 11, 13 or 29 atoms in the chain.

44. The conjugate of claim 41, wherein the linker comprises straight alkelene chain containing from 1 up to 50 carbon atoms in the chain.

45. The conjugate of claim 1, wherein the linker comprises straight alkelene chain containing 2, 3, 4, 5, 6, 7, 8, 9, 10, 11 or 12 carbon atoms in the alkelene chain.

46. The conjugate of claim 1, wherein the linker comprises straight alkelene chain containing 3, 4, 5, 6, 7, 8 or 9 carbon atoms in the alkelene chain.

47. The conjugate of claim 1 having formula
(D)-(L)-(S), or a pharmaceutically acceptable derivative thereof, wherein
D is a drug moiety; L is a non-releasable linker; and S is a substrate for a kinase other than a hexokinase, a protein kinase or a lipid kinase.

48. The conjugate of claim 47 having formula
(D)-(L)-(N), or a pharmaceutically acceptable derivative thereof, wherein
D is a drug moiety; L is a non-releasable linker; and N is a natural or non-natural nucleoside.

49. The conjugate of claim 48 having formula
S_{c}-P¹-L-D,
or a pharmaceutically acceptable derivative thereof, wherein S_{c} is ribose, deoxyribose or analog thereof and P¹ is a purine, pyrimidine or analog thereof.

50. The conjugate of claim 48 having formula
P¹-S_{c}-L-D,
or a pharmaceutically acceptable derivative thereof, wherein S_{c} is ribose, deoxyribose or analog thereof, P¹ is a purine, pyrimidine or analog thereof.

51. The conjugate of claim 48 having formula 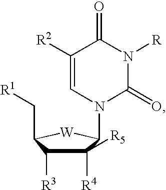
or a pharmaceutically acceptable derivative thereof, wherein
R¹, R³, R⁴ and R⁵ are each independently Y, H, hydroxy, halo, azido, C1-6 alkyl and optionally containing a heteroatom, C2-6 alkenyl or C2-6 alkynyl;
R² is Y, H, C1-6 alkyl and optionally containing a heteroatom, C2-6 alkenyl, C2-6 alkynyl, C3-6 cycloalkyl, aryl, heteroaryl or halo;
R is Y, H or C1-6 alkyl, C2-6 alkenyl or C2-6 alkynyl;
W is CR^{e}R^{f} or O; R^{e} and R^{f} are each independently H or C1-6 alkyl;
Y is L-D, wherein L, which may or may not be present, is a non-releasable linker and D a drug moiety;
R and R¹-R⁵ are selected such that at least one of R and R′-R⁵ is Y and at least one of R¹ and R³-R⁵ is OH;
R¹ and R²-R⁵ and R are unsubstituted or substituted with 1-4 substituents, each independently selected from Q¹,
Q¹ is halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q¹ groups, which substitute atoms in a 1, 2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹ groups, which substitute the same atom, together form alkylene; and
each Q¹ is independently unsubstituted or substituted with one, two or three substituents, each independently selected from Q²;
each Q² is independently halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1, 2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;
R⁵⁰ is hydroxy, alkoxy, aralkoxy, alkyl, heteroaryl, heterocyclyl, aryl or —NR⁷⁰R⁷¹, where R⁷⁰ and R⁷¹ are each independently hydrogen, alkyl, aralkyl, aryl, heteroaryl, heteroaralkyl or heterocyclyl, or R⁷⁰ and R⁷¹ together form alkylene, azaalkylene, oxaalkylene or thiaalkylene;
R⁵¹, R⁵² and R⁵³ are each independently hydrogen, alkyl, aryl, aralkyl, heteroaryl, heteroaralkyl, heterocyclyl or heterocyclylalkyl;
R⁶⁰ is hydrogen, alkyl, aryl, aralkyl, heteroaryl, heteroaralkyl, heterocyclyl or heterocyclylalkyl; and
R⁶³ is alkoxy, aralkoxy, alkyl, heteroaryl, heterocyclyl, aryl or —NR⁷⁰R⁷¹.

52. The conjugate of claim 51, wherein R¹ is Y, H, hydroxy, halo, azido, C1-6 alkyl and optionally containing a heteroatom, C2-6 alkenyl or C2-6 alkynyl.

53. The conjugate of claim 51, wherein R¹ is OH.

54. The conjugate of claim 51, wherein R³ is hydroxy.

55. The conjugate of claim 51, wherein R³ is Y.

56. The conjugate of claim 51, wherein R⁴ is hydroxy.

57. The conjugate of claim 51, wherein R⁴ is H.

58. The conjugate of claim 51, wherein R⁴ is Y.

59. The conjugate of claim 51, wherein R⁵ is H.

60. The conjugate of claim 51, wherein R⁵ is hydroxy.

61. The conjugate of claim 51, wherein R⁵ is Y.

62. The conjugate of claim 51, wherein R² is H.

63. The conjugate of claim 51, wherein R² is C1-6 alkyl.

64. The conjugate of claim 51, wherein R² is methyl.

65. The conjugate of claim 51, wherein R² is Y.

66. The conjugate of claim 51, wherein R² is halo

67. The conjugate of claim 51, wherein R is C1-6 alkyl.

68. The conjugate of claim 51, wherein R is Y.

69. The conjugate of claim 51, wherein W is CR^{e}R^{f} or O.

70. The conjugate of claim 51, wherein W is O.

71. The conjugate of claim 51, wherein W is CR^{e}R^{f}.

72. The conjugate of claim 51, wherein R^{e} and R^{f} are each H.

73. The conjugate of claim 51, wherein Y is -L-D

74. The conjugate of claim 51, wherein Y is D.

75. The conjugate of claim 51, wherein -L- is selected from a bifunctional alkelene chain or bifunctional polyethylene glycol chain.

76. The conjugate of claim 51, wherein -L- is —O-(L₁)-, where L₁ is non-releasable linker.

77. The conjugate of claim 51, wherein -L₁- is selected from a bifunctional alkelene chain or bifunctional polyethylene glycol chain.

78. The conjugate of claim 51, wherein the conjugate has formula: 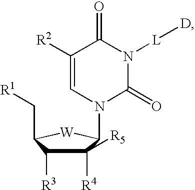
or a pharmaceutically acceptable derivative thereof,
wherein, R¹ and R³ are Hydroxy; R⁴ is H; R⁵ is H or hydroxy; R² is H or C1-6 alkyl; and W is O.

79. The conjugate of claim 51, wherein the conjugate has formula: 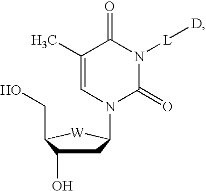
or a pharmaceutically acceptable derivative thereof.

80. The conjugate of claim 1, wherein the conjugate has formula: 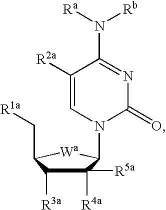
or a pharmaceutically acceptable derivative thereof,
wherein
R^{1a}, R^{3a}, R^{4a} and R^{5a} are each independently Y; H, hydroxy, halo, azido, C1-6 alkyl and optionally containing a heteroatom, C2-6 alkenyl or C2-6 alkynyl;
R^{2a} is Y, H, C1-6 alkyl and optionally containing a heteroatom, C2-6 alkenyl, C2-6 alkynyl, C3-6 cycloalkyl, aryl or heteroaryl;
Y is L-D, wherein L, which may or may not be present, is a non-releasable linker and D is a drug moiety;
R^{1a}-R^{5a} are selected such that at least one of R^{1a}-R^{5a} is Y and at least one of R^{1a}, R^{3a}-R^{5a} is OH;
R^{a} and R^{b} are each independently Y, H, or C1-6 alkyl;
R^{d} is H or C1-6 alkyl;
W^{a} is CR^{e}R^{f} or O; R^{e} and R^{f} are each independently H or C1-6 alkyl;
R^{1a}-R^{5a}, R^{a}, R^{b} and R^{d} are unsubstituted or substituted with 1-4 substituents selected from Q¹
each Q¹ is independently unsubstituted or substituted with one, two or three substituents, each independently selected from Q²;
each Q² is independently halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1, 2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;
R⁵⁰ is hydroxy, alkoxy, aralkoxy, alkyl, heteroaryl, heterocyclyl, aryl or —NR⁷⁰R⁷¹, where R⁷⁰ and R⁷¹ are each independently hydrogen, alkyl, aralkyl, aryl, heteroaryl, heteroaralkyl or heterocyclyl, or R⁷⁰ and R⁷¹ together form alkylene, azaalkylene, oxaalkylene or thiaalkylene;
R⁵¹, R⁵² and R⁵³ are each independently hydrogen, alkyl, aryl, aralkyl, heteroaryl, heteroaralkyl, heterocyclyl or heterocyclylalkyl;
R⁶⁰ is hydrogen, alkyl, aryl, aralkyl, heteroaryl, heteroaralkyl, heterocyclyl or heterocyclylalkyl; and
R⁶³ is alkoxy, aralkoxy, alkyl, heteroaryl, heterocyclyl, aryl or —NR⁷⁰R⁷¹.

81. The conjugate of claim 80, wherein R^{1a} is OH and W=O.

82. The conjugate of claim 1, wherein the conjugate has formula: 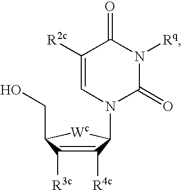
or a pharmaceutically acceptable derivative thereof, wherein
R³′ and R^{4c} are each independently Y, H, C1-6 alkyl and optionally containing a heteroatom, C2-6 alkenyl or C2-6 alkynyl;
R^{2c} is Y, H, C1-6 alkyl and optionally containing a heteroatom, C2-6 alkenyl, C2-6 alkynyl, C3-6 cycloalkyl, aryl, heteroaryl or halo;
R^{q} is Y, H or C1-6 alkyl;
Y is L-D, wherein L, which may or may not be present, is a non-releasable linker and D is a drug moiety;
W^{c} is CR^{e}R^{f} or O; R^{e} and R^{f} are each independently H or C1-6 alkyl;
R^{3c}-R^{4c} and R^{q} are selected such that at least one of R^{1c}-R^{4c} or R^{q} is Y;
R^{3c}-R^{2c} and R^{q} are unsubstituted or substituted with 1-4 substituents selected from Q¹
each Q¹ is independently unsubstituted or substituted with one, two or three substituents, each independently selected from Q²;
each Q² is independently halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1, 2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;
R⁵⁰ is hydroxy, alkoxy, aralkoxy, alkyl, heteroaryl, heterocyclyl, aryl or —NR⁷⁰R⁷¹, where R⁷⁰ and R⁷¹ are each independently hydrogen, alkyl, aralkyl, aryl, heteroaryl, heteroaralkyl or heterocyclyl, or R⁷⁰ and R⁷¹ together form alkylene, azaalkylene, oxaalkylene or thiaalkylene;
R⁵¹, R⁵² and R⁵³ are each independently hydrogen, alkyl, aryl, aralkyl, heteroaryl, heteroaralkyl, heterocyclyl or heterocyclylalkyl;
R⁶⁰ is hydrogen, alkyl, aryl, aralkyl, heteroaryl, heteroaralkyl, heterocyclyl or heterocyclylalkyl; and
R⁶³ is alkoxy, aralkoxy, alkyl, heteroaryl, heterocyclyl, aryl or —NR⁷⁰R⁷¹.

83. The conjugate of claim 1, wherein the conjugate has formula: 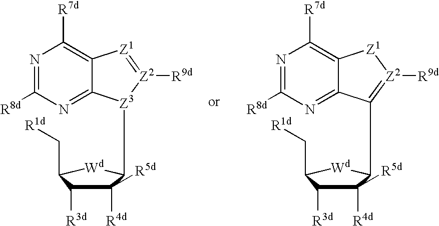
or a pharmaceutically acceptable derivative thereof, wherein
R^{1d}, R^{3d}, R^{4d} and R^{5d} are each independently Y, H, hydroxy, halo, azido, C1-6 alkyl and optionally containing a heteroatom, C2-6 alkenyl or C2-6 alkynyl;
R^{7d} is Y, H, hydroxy, halo, azido, C1-6 alkyl and optionally containing a heteroatom, C2-6 alkenyl, C2-6 alkynyl, NR^{a}R^{b} or SR^{d};
R^{8d} is Y, H, halo or NR^{a}R^{b};
R^{9d} is Y. H, or C1-6 alkyl;
Y is L-D, wherein L, which may or may not be present, is a non-releasable linker and D is a drug moiety;
R^{1d}-R^{9d} are selected such that at least one of R^{1d}, R^{3d}, R^{4d}, R^{5d} or R^{7d} is Y and at least one of R^{1d}, R^{3d}, R^{4d}, R^{5d} or R^{7d} is OH;
R^{a}, R^{b} and R^{d} are each independently Y, H, or C1-6 alkyl;
R^{d} is H or C1-6 alkyl;
W^{d} is CR^{e}R^{f} or O; R^{e} and R^{f} are each independently H or C1-6 alkyl;
Z¹, Z² and Z³ are each independently C or N;
R^{1d}-R^{9d}, R^{a}, R^{b} and R^{d} are unsubstituted or substituted with 1-4 substituents selected from Q¹
each Q¹ is independently unsubstituted or substituted with one, two or three substituents, each independently selected from Q²;
each Q² is independently halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1, 2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;
R⁵⁰ is hydroxy, alkoxy, aralkoxy, alkyl, heteroaryl, heterocyclyl, aryl or —NR⁷⁰R⁷¹, where R⁷⁰ and R⁷¹ are each independently hydrogen, alkyl, aralkyl, aryl, heteroaryl, heteroaralkyl or heterocyclyl, or R⁷⁰ and R⁷¹ together form alkylene, azaalkylene, oxaalkylene or thiaalkylene;
R⁵¹, R⁵² and R⁵³ are each independently hydrogen, alkyl, aryl, aralkyl, heteroaryl, heteroaralkyl, heterocyclyl or heterocyclylalkyl;
R⁶⁰ is hydrogen, alkyl, aryl, aralkyl, heteroaryl, heteroaralkyl, heterocyclyl or heterocyclylalkyl; and
R⁶³ is alkoxy, aralkoxy, alkyl, heteroaryl, heterocyclyl, aryl or —NR⁷⁰R⁷¹.

84. The conjugate of claim 83, wherein R^{1d} is OH, W^{d} is O and R^{9d} is Y.

85. The conjugate of claim 1, wherein the conjugate has formula: 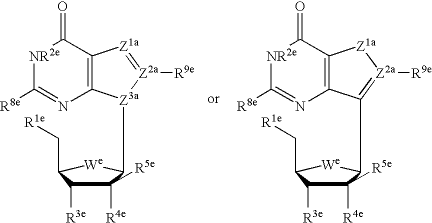
or a pharmaceutically acceptable derivative thereof, R^{1e}, R^{3e}, R^{4e} and R^{5e} are each independently Y, H, hydroxy, halo, azido, C1-6 alkyl and optionally containing a heteroatom, C2-6 alkenyl or C2-6 alkynyl;
R^{2e} is Y, H, C1-6 alkyl and optionally containing a heteroatom, C2-6 alkenyl, C2-6 alkynyl or C3-6 cycloalkyl;
R^{8e} is Y, H, halo or NR^{a}R^{b} or SR^{d};
R^{9e} is Y, H, or C1-6 alkyl;
Y is L-D, wherein L, which may or may not be present, is a non-releasable linker and D is a drug moiety;
R^{a}, R^{b} and R^{d} are each independently Y, H, or C1-6 alkyl;
R^{1e}-R^{5e}, R^{8e} and R^{9e} are selected such that at least one of R^{1e}-R^{5e}, R^{8e} and R^{9e} is Y and at least one of R^{1e}, R^{3e}, R^{4e} and R^{5e} is OH;
W^{e} is CR^{e}R^{f} or O; R^{e} and R^{f} are each independently H or C1-6 alkyl;
Z^{1a}, Z^{2a} and Z^{3a} are each independently C or N;
R^{1e}-R^{5e}, R^{8e} and R^{9e} are unsubstituted or substituted with 1-4 substituents selected from Q¹
each Q¹ is independently unsubstituted or substituted with one, two or three substituents, each independently selected from Q²;
each Q² is independently halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1, 2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;
R⁵⁰ is hydroxy, alkoxy, aralkoxy, alkyl, heteroaryl, heterocyclyl, aryl or —NR⁷⁰R⁷¹, where R⁷⁰ and R⁷¹ are each independently hydrogen, alkyl, aralkyl, aryl, heteroaryl, heteroaralkyl or heterocyclyl, or R⁷⁰ and R⁷¹ together form alkylene, azaalkylene, oxaalkylene or thiaalkylene;
R⁵¹, R⁵² and R⁵³ are each independently hydrogen, alkyl, aryl, aralkyl, heteroaryl, heteroaralkyl, heterocyclyl or heterocyclylalkyl;
R⁶⁰ is hydrogen, alkyl, aryl, aralkyl, heteroaryl, heteroaralkyl, heterocyclyl or heterocyclylalkyl; and
R⁶³ is alkoxy, aralkoxy, alkyl, heteroaryl, heterocyclyl, aryl or —NR⁷⁰R⁷¹.

86. The conjugate of claim 85, wherein R^{1e} is OH, W^{e} is O and R^{9e} is Y.

87. The conjugate of claim 1, wherein the conjugate has formula: 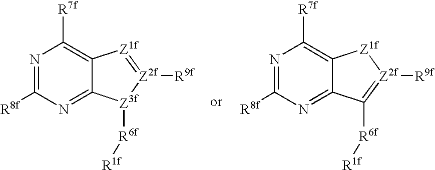
or a pharmaceutically acceptable derivative thereof, wherein, R^{6f} is C1-10 alkyl and optionally containing a heteroatom, C2-10 alkenyl or C2-10 alkynyl;
R^{1f} is Y or hydroxy;
R^{7f} is Y, H, hydroxy, halo, azido, C1-6 alkyl and optionally containing a heteroatom, C2-6 alkenyl, C2-6 alkynyl, NR^{a}R^{b} or SR^{d};
R^{8f} is Y, H, halo or NR^{a}R^{b} or SR^{d};
R^{9e} is Y, H, or C1-6 alkyl;
Y is L-D, wherein L, which may or may not be present, is a non-releasable linker and D is a drug moiety;
R^{7f}, R^{8f} and R^{9f} are selected such that at least one of R^{1f}, R^{7f}, R^{8f} and R^{9f} is Y and at least one of R^{7f} and R^{1f} is OH;
R^{a}, R^{b} and R^{d} are each independently Y, H, or C1-6 alkyl;
Z^{1f}Z^{2f} and Z^{3f} are each independently C or N;
R^{7f}, R^{8f} and R^{9f} are unsubstituted or substituted with 1-4 substituents selected from Q¹
each Q¹ is independently unsubstituted or substituted with one, two or three substituents, each independently selected from Q²;
each Q² is independently halo, pseudohalo, hydroxy, oxo, thia, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1, 2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;
R⁵⁰ is hydroxy, alkoxy, aralkoxy, alkyl, heteroaryl, heterocyclyl, aryl or —NR⁷⁰R⁷¹, where R⁷⁰ and R⁷¹ are each independently hydrogen, alkyl, aralkyl, aryl, eteroaryl, heteroaralkyl or heterocyclyl, or R⁷⁰ and R⁷′ together form alkylene, azaalkylene, oxaalkylene or thiaalkylene;
R⁵¹, R⁵² and R⁵³ are each independently hydrogen, alkyl, aryl, aralkyl, eteroaryl, heteroaralkyl, heterocyclyl or heterocyclylalkyl;
R⁶⁰ is hydrogen, alkyl, aryl, aralkyl, heteroaryl, heteroaralkyl, heterocyclyl or heterocyclylalkyl; and
R⁶³ is alkoxy, aralkoxy, alkyl, heteroaryl, heterocyclyl, aryl or —NR⁷⁰R⁷¹.

88. The conjugate of claim 1, wherein the conjugate has an improved cytotoxic selectivity index as compared to an unconjugated drug

89. The conjugate of claim 1, wherein the cytotoxic selectivity index is more than about 1.5 folds up to more than about 100 folds improved.

90. A method of treatment of conditions caused by ACAMPS comprising administering to a subject an effective amount of the conjugate of claim 1, or a pharmaceutically acceptable derivative thereof.

91. The method of claim 90, wherein the ACAMPS condition is characterized by undesirable or aberrant activation, migration, proliferation or survival of tumor cells, endothelial cells, B cells, T cells, macrophages, neutrophils, eosinophils, basophils, monocytes, platelets, fibroblasts, other connective tissue cells, osteoblasts, osteoclasts and progenitors of these cell types.

92. The method of claim 91, wherein the ACAMPS condition is a cancer, coronary restenosis, osteoporosis, chronic inflammation or an autoimmunity disease.

93. The method of claim 92, wherein the autoimmune disease is rheumatoid arthritis, asthma, psoriasis, inflammatory bowel disease, systemic lupus erythematosus, systemic dermatomyositis, inflammatory ophthalmic diseases, autoimmune hematologic disorders, multiple sclerosis, vasculitis, idiopathic nephrotic syndrome, transplant rejection or graft versus host disease.

94. The method of claim 92, wherein the cancer is non-small cell lung cancer, small cell lung cancer, head squamous cancer, neck squamous cancer, colorectal cancer, prostate cancer, breast cancer, acute lymphocytic leukemia, adult acute myeloid leukemia, adult non-Hodgkin's lymphoma, brain tumor, cervical cancer, childhood cancer, childhood sarcoma, chronic lymphocytic leukemia, chronic myeloid leukemia, esophageal cancer, hairy cell leukemia, kidney cancer, liver cancer, multiple myeloma, neuroblastoma, oral cancer, pancreatic cancer, primary central nervous system lymphoma, skin cancer or small-cell lung cancer.

95. The method of claim 92, wherein the cancer is brain stem glioma, cerebellar astrocytoma, cerebral astrocytoma, ependymoma, Ewing's sarcoma, germ cell tumor, Hodgkin's disease, ALL, AML, liver cancer, medulloblastoma, neuroblastoma, non-Hodgkin's lymphoma, osteosarcoma, malignant fibrous histiocytoma of bone, retinoblastoma, rhabdomyosarcoma, soft tissue sarcoma, supratentorial primitive neuroectodermal and pineal tumors, visual pathway and hypothalamic glioma, Wilms' tumor, or other childhood kidney tumor.

96. The method of claim 92, wherein the cancer is originated from or have metastasized to the bone, brain, breast, digestive and gastrointestinal system, endocrine system, blood, lung, respiratory system, thorax, musculoskeletal system, or skin.

97. The method of claim 92, wherein the cancer is selected from breast cancer, lung cancer, prostate cancer, ovarian cancer, esophageal cancer, bladder cancer, hepatoma, neuroblastoma, lymphoma, testicular cancer, renal cancer, leukemia, colorectal cancer and head and neck cancer.

98. A method for identifying kinase substrates capable of selectively accumulating in a target system, comprising the steps of:
a) contacting one or more conjugate of claim 1 with a kinase that is overexpressed, overactive or that exhibits undesired activity in a target system;
b) determining kinase activity on the one or more conjugate.

99. A method of claim 98 further comprising the steps of
c) determining a first amount or a plurality of first amounts of the conjugates in the target system;
d) determining a second amount or a plurality of second amounts of the one or more conjugates in a non-target system.

100. The method of claim 98, wherein the one or more conjugates comprises a detectable label.

101. The method of claim 100, wherein the label is a radioactive or fluorescent label.

102. The method of claim 98, wherein the target system is associated with an ACAMPS condition.

103. The method of claim 98, wherein the target system is associated cancer, inflammation, angiogenesis, autoimmune syndromes, transplant rejection or osteoporosis.

104. The method of claim 98, wherein the target system is a cell.

105. The method of claim 104, wherein the cell is a tumor cell or a tumor-associated endothelial cell.

106. A method for identifying conjugates capable of exhibiting selective toxicity against a target system, comprising:
a) contacting one or more conjugates of claim 1 with a target system; and
b) determining the cytotoxicity of the one or more conjugates against the target system.

107. The method of claim 106, wherein the target system is associated with cancer, inflammation, angiogenesis, autoimmune syndromes, transplant rejection or osteoporosis.

108. The method of claim 107, wherein the target system is a cell.

109. The method of claim 107, wherein the cell is a tumor cell or a tumor-associated endothelial cell.

110. The method of claim 107, wherein the drug moiety is an anti-cancer drug.

111. A method of enhancing drug efficiency, comprising administering to a target system, or organism a therapeutically effective amount of the conjugate of claim 1, thereby improving drug efficiency as compared to an unconjugated drug.

112. The conjugate of claim 1 having formula: 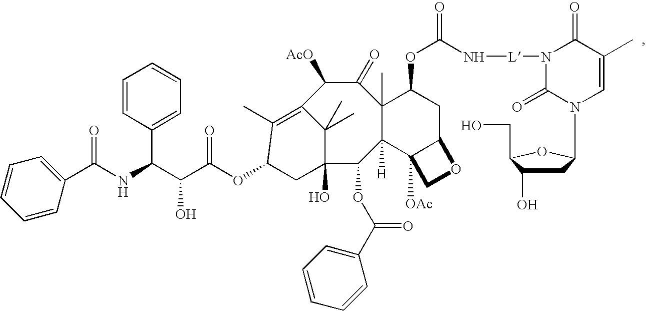
or a pharmaceutically acceptable derivative thereof, where L′ is alkylene or PEG.

113. The conjugate of claim 1 having formula: 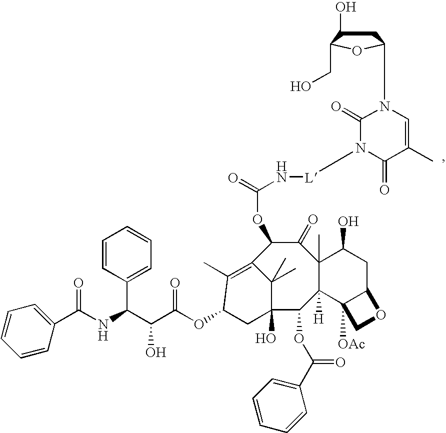
or a pharmaceutically acceptable derivative thereof, where L′ is alkylene or PEG.

114. The conjugate of claim 1 having formula: 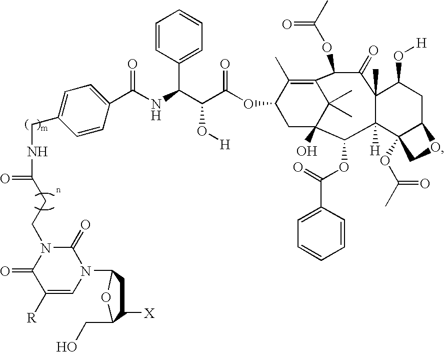
or a pharmaceutically acceptable derivative thereof, where n and m are each independently 1-10.

115. The conjugate of claim 1 having formula: 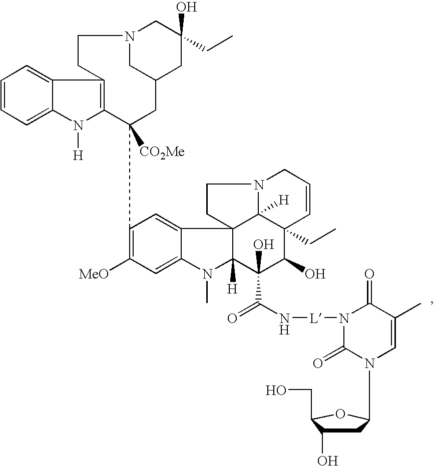
or a pharmaceutically acceptable derivative thereof, where L′ is alkylene or PEG.

116. The conjugate of claim 1 having formula: 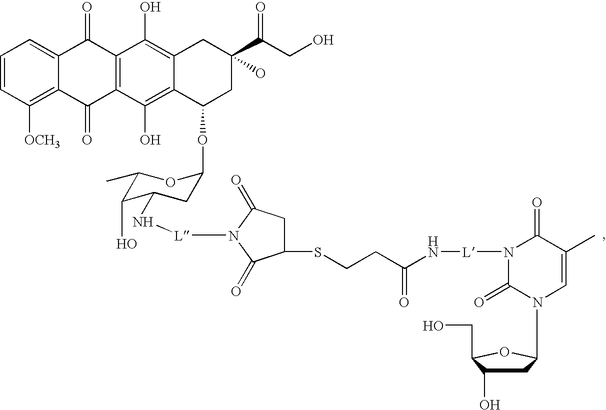
or a pharmaceutically acceptable derivative thereof, where L′ and L″ are each independently alkylene or PEG.

117. The conjugate of claim 1 having formula:

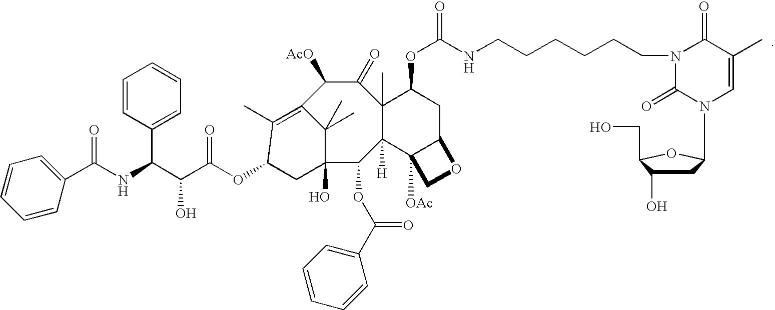

118. The conjugate of claim 1 selected from Tables 4-6.

119. An article of manufacture, comprising packaging material, the conjugate of claim 1, or a pharmaceutically acceptable derivative thereof, contained within packaging material, which is used for treatment, prevention or amelioration of one or more symptoms associated with ACAMPS, and a label that indicates that the compound or pharmaceutically acceptable derivative thereof is used for treatment, prevention or amelioration of one or more symptoms associated with ACAMPS.

120. A pharmaceutical composition comprising a conjugate of claim 1 in a pharmaceutically acceptable carrier.

121. The conjugate of claim 1, wherein the substrate is a natural or a non-natural nucleoside base that is converted to a substrate of thymidine kinase or deoxycytidine kinase by an action of thymidine phosphorylase or cytidine deaminase.


SEQUENCE LISTING (2 sequences)

SEQ ID NO: 1 - DNA, 27 bp, Artificial Sequence
  Forward primer
caatccatatgagctgcattaacctgc

SEQ ID NO: 2 - DNA, 29 bp, Artificial Sequence
  Reverse primer
tattaagcttctagttggcagggctgcat